(19) United States
(12) Patent Application Publication
(10) Pub. No.: US 2022/0106288 A1
(43) Pub. Date: Apr. 7, 2022

(54) NOVEL SULFONEUREA COMPOUNDS

(71) Applicant: INFLAZOME LIMITED, Dublin 2 (IE)

(72) Inventors: Angus MACLEOD, Cambridge (GB); Jonathan SHANNON, Nottingham (GB); Stephen THOM, Nottingham (GB); Jokin CARRILLO ARREGUI, Nottingham (GB); Thomas ALANINE, Nottingham (GB)

(21) Appl. No.: 17/267,781

(22) PCT Filed: Aug. 12, 2019
(86) PCT No.: PCT/EP2019/071630

§ 371 Date: Feb. 10, 2021

(30) Foreign Application Priority Data
Aug. 15, 2018 (EP) PCT/EP2018/072111
Feb. 20, 2019 (GB) 1902327.4

(51) Int. Cl. C07D 401/12; C07D 213/40; C07D 213/64; C07D 213/647; C07D 405/12; C07D 401/14; C07D 405/14; C07D 401/04; A61P 37/06

(52) U.S. Cl. CPC C07D 401/12; C07D 213/40; C07D 213/64; C07D 213/647; C07B 2200/05; C07D 401/14; C07D 405/14; C07D 401/04; A61P 37/06; C07D 405/12

(57) ABSTRACT

The present invention relates to compounds of formula (I): wherein A, B, X, Y, R¹, R⁴ and R⁷ are as defined in the specification. The present invention further relates to salts, solvates and prodrugs of such compounds, to pharmaceutical compositions comprising such compounds, and to the use of such compounds in the treatment and prevention of medical disorders and diseases, most especially by the inhibition of NLRP₃.

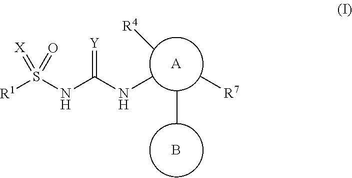

### FIELD OF THE INVENTION

The present invention relates to compounds of formula (I), and to associated salts, solvates, prodrugs and pharmaceutical compositions. The present invention further relates to the use of such compounds in the treatment and prevention of medical disorders and diseases, most especially by NLRP3 inhibition.

### BACKGROUND OF THE INVENTION

The NOD-like receptor (NLR) family, pyrin domain—containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Thi response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al., J Pharmacol Exp Ther, 299: 187-197, 2001). CRIDs are a class of diarylsulfonylurea-containing compounds that inhibit the post-translational processing of IL-1β. Post-translational processing of IL-1β is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184623 A1, WO 2017/184624 A1, WO 2018/015445 A1, WO 2018/136890 A1, WO 2018/215818 A1, WO 2019/008025 A1, WO 2019/008029 A1, WO 2019/034686 A1, WO 2019/034688 A1, WO 2019/034690 A1, WO 2019/034692 A1, WO 2019/034693 A1, WO 2019/034696 A1, WO 2019/034697 A1, WO 2019/043610 A1, WO 2019/092170 A1, WO 2019/092171 A1, and WO 2019/092172 A1). In addition, WO 2017/184604 A1 and WO 2019/079119 A1 disclose a number of sulfonylamide-containing compounds as inhibitors of NLRP3.

Certain sulfoximine-containing compounds are also disclosed as inhibitors of NLRP3 (WO 2018/225018 A1, WO 2019/023145 A1, WO 2019/023147 A1, and WO 2019/068772 A1).

There is a need to provide compounds with improved pharmacological and/or physiological and/or physicochemical properties and/or those that provide a useful alternative to known compounds.

Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkoxyalkyl” substituent group or an alkoxyalkyl moiety in a substituent group is an (alkyl)-O-(alkylene)- group. Typically an alkoxyalkyl group is a (C₁-C₆ alkyl)-O—(C₁-C₆ alkylene)- group. More typically an alkoxyalkyl group is a (C₁-C₄ alkyl)-O—(C₁-C₆ alkylene)- group. More typically an alkoxyalkyl group is a (C₁-C₃ alkyl)-O—(C₁-C₆ alkylene)- group. Examples of alkoxyalkyl groups/moieties include methoxyalkyl and ethoxyalkyl. Examples of methoxyalkyl include methoxy-(C₁-C₆ alkylene)-, methoxy-(C₁-C₄ alkylene)- and methoxy-(C₁-C₃ alkylene)-. Examples of ethoxyalkyl include ethoxy-(C₁-C₆ alkylene)-, ethoxy-(C₁-C₄ alkylene)- and ethoxy-(C₁-C₃ alkylene) .

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl.

Unless stated otherwise, a cycloalkenyl substituent group or moiety may include monocycle, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

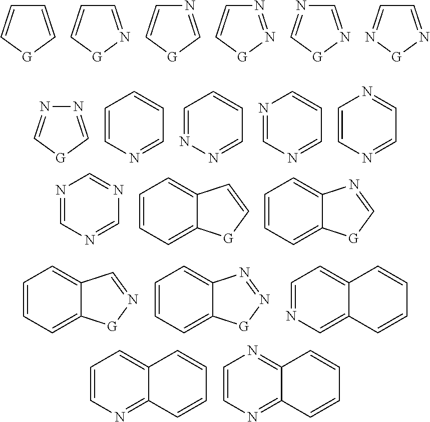

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety:

(i) each hydrogen atom may optionally be replaced by a monovalent substituent independently selected from halo; —CN; —NO₂; —N₃; R^{β}; —OH; —OR^{β}; —R^{α}-halo; —R^{α}—CN; —R^{α}—NO₂; —R^{α}—N_{β}; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})³; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —NH—COOR^{β}; —NR^{β}—COOR^{β}; —NH—C(═NH)R^{β}; —NR^{β}—C(═NH)R^{β}; —NH—C(═NH)NH₂; —NR^{β}—C(═NH)NH₂; —NH—C(═NH)NHR^{β}; —NR^{β}—C(═NH)NHR^{β}; —NH—C(═NH)N(R^{β})₂; —NR^{β}—C(═NH)N(R^{β})₂; —NH—C(═NR^{β})R^{β}; —NR^{β}—C(═NR^{β})R^{β}; —NH—C(═NR^{β})NHR^{β}; —NR^{β}—C(═NR^{β})NHR^{β}; —NH—C(═NR^{β})N(R^{β})₂; —NR^{β}—C(═NR^{β})N(R^{β})₂; —NH—C(═NOH)R^{β}; —NR^{β}—C(═NOH)R^{β}; —NH—C(═NOR^{β})R^{β}; —NR^{β}—C(═NOR^{β})R^{β}; —CONH₂; —CONHR^{β}; —CON(R^{β})₂; —NH—CONH₂; —NR^{β}—CONH₂; —NH—CONHR^{β}; —NR^{β}—CONHR^{β}; —NH—CON(R^{β})₂; —NR^{β}—CON(R^{β})₂; —R^{α}—NH—CHO; —R^{α}—NR^{β}—CHO; —R^{α}—NH—COR^{β}; —R^{α}—NR^{β}—COR^{β}; —R^{α}—NH—COOR^{β}; —R^{α}—NR^{β}—COOR^{β}; —R^{α}—NH—C(═NH)R^{β}; —R^{α}—NR^{β}—C(═NH)R^{β}; —R^{α}—NH—C(═NH)NH₂; —R^{α}—NR^{β}—C(═NH)NH₂; —R^{α}—NH—C(═NH)NHR^{β}; —R^{α}—NR^{β}—C(═NH)NHR^{β}; —R^{α}—NH—C(═NH)N(R^{β})₂; —R^{α}—NR^{β}—C(═NH)N(R^{β})₂; —R^{α}—NH—C(═NR¹³)R¹³; —R^{α}—NR^{β}—C(═NR^{β})R^{β}; —R^{α}—NH—C(═NR^{β})NHR^{β}; —R^{α}—NR^{β}—C(═NR^{β})NHR^{β}; —R^{α}—NH—C(═NR^{β})N(R^{β})₂; —R^{α}—NR^{β}—C(═NR^{β})N(R^{β})₂; —R^{α}—NH—C(═NOH)R^{β}; —R^{α}—NR^{β}—C(═NOH)R^{β}; —R^{α}—NH—C(═NOH^{β})R^{β}; —R^{α}—NR^{β}—C(═NOR^{β})R^{β}; —R^{α}—CONH₂; —R^{α}—CONHR^{β}; —R^{α}—CON(R^{β})₂; —R^{α}—NH—CONH₂; —R^{α}—NR^{β}—CONH₂; —R^{α}—NH—CONHR^{β}; —R^{α}—NR^{β}—CONHR¹³; —R^{α}—NH—CON(R¹³)₂; —R^{α}—NR^{β}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —R^{β}—R^{α}—NH₂; —NR^{β}—R^{α}—NHR^{β}; —NR^{β}—R^{α}—N(R^{β})₂; —NR⁶² —R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon or nitrogen atom may optionally be replaced by a π-bonded substituent independently selected from oxo (═O), ═S, ═NH or ═NR^{β}; and/or

(iii) any sulfur atom may optionally be substituted with one or two π-bonded substituents independently selected from oxo (═O), ═NH or ═NR^{β}; and/or

(iv) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C≡CH, oxo (═O), phenyl, halophenyl, or optionally halo-substituted 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

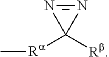

Typically, in an optionally substituted group or moiety:

(i) each hydrogen atom may optionally be replaced by a monovalent substituent independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —N+(R^{β})₃; —R^{α}—NH₂; —R^{α}—NHR^{β}; —R^{α}—N(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or

(ii) any two hydrogen atoms attached to the same carbon atom may optionally be replaced by a π-bonded substituent independently selected from oxo (═O), ═S, ═NH or ═NR^{β}; and/or

(iii) any two hydrogen atoms attached to the same or different atoms, within the same optionally substituted group or moiety, may optionally be replaced by a bridging substituent independently selected from —O—, —S—, —NH—, —N(R^{β})—, —N⁺(R^{βl )}₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein a single —CH₂— group in the backbone of the alkylene, alkenylene or alkynylene group may optionally be replaced by a —N⁺(R^{β})₂— group, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), ^{—O(C}₁-C₄ haloalkyl), —O(C₃-C₇ cycloalkyl), halo, —OH, —NH₂, —CN, —C≡CH, oxo (═O), or 4- to 6-membered heterocyclic group.

Typically, in an optionally substituted group or moiety:

(i) each hydrogen atom may optionally be replaced by a monovalent substituent independently selected from halo; —CN; —NO₂; —N₃; —R^{β}; —OH; —OR^{β}; —R^{α}—halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR¹³; —R^{α}—SO₂N(R¹³)₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}COR^{β}; —R^{α}—COOH; —R^{α}—COOR^{β}; or —R^{α}—OCOR^{β}; and/or

(ii) any two hydrogen atoms attached to the same carbon atom may optionally be replaced by a π-bonded substituent independently selected from oxo (═O), ═S, ═NH or ═NR^{β}; and/or

(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two —R^{β} attached to the same nitrogen atom may, together with the nitrogen atom to which they are attached, form a C₂-C₆ cyclic group, and wherein any —R^{β} may optionally be substituted with one or more C₁-C₄ alkyl, halo, —OH, or 4- to 6-membered heterocyclic group.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

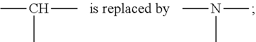

—CH₂—is replaced by —NH—, —O—or —S—;
—CH₃ is replaced by —NH₂, —OH or —SH;
—CH═ is replaced by —N═;
CH₂═ is replaced by NH═, O═or S═; or
CH≡ is replaced by N≡;

provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group. what is intended is that:
—CH₂—is replaced by or

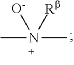

—CH₂—is replaced by

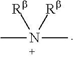

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are to be counted as carbon atoms when calculating the number of carbon atoms in a Cₓ-C_{y} group. For example, a morpholinyl group is to be considered a C₆ heterocyclic group, not a C₄ heterocyclic group.

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

For the avoidance of doubt, where it is stated that a compound or a group, such as R¹, contains from x to y atoms other than hydrogen, it is to be understood that the compound or group as a whole, including any optional substituents, contains from x to y atoms other than hydrogen. Such a compound or group may contain any number of hydrogen atoms.

### SUMMARY OF THE INVENTION

A first aspect of the invention provides a compound of formula (I):

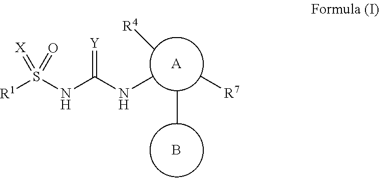

wherein:
A is a phenyl or 5- or 6-membered heteroaryl group, wherein A is substituted in the α position with B, in the β position with R⁷ and in the α′ position with R⁴, and wherein A is optionally further substituted;
B is a phenyl, 5- or 6-membered heteroaryl, or 4- to 6-membered saturated heterocyclic group, wherein B is optionally substituted;
X is O, NH or N(CN);
Y is O or S;
R¹ is a C₁-C₄ alkyl, C₂-C₄ alkenyl, —NH(C₁-C₄ alkyl), —-N(C₁-C₄ alkyl)₂, or —R²⁰—R²¹ group, all optionally halo-substituted;
either R⁴ is monovalent, and attached to A in the α′ position, and selected from C₁-C₄ alkyl, C₃-C₆ cycloalkyl and phenyl, all optionally halo-substituted and/or optionally substituted with one or two substituents independently selected from oxo, —OH, —O(C₁-C₄ alkyl) and —O(C₁-C₄ haloalkyl);
or R⁴ is divalent, and attached to A in the α′ and β′ positions, and selected from —CH₂CH₂CH₂—, —CH═CHCH₂—, —CH₂CH═CH—, —CH₂CH₂O—, —OCH₂CH₂—, —CH₂CH₂CH₂CH₂—and —CH═CH—CH═CH—, all optionally halo-substituted and/or optionally substituted with one or two substituents independently selected from oxo, —OH, —O(C₁-C₄ alkyl) and —O(C₁-C₄ haloalkyl);
R⁷ is C₁-C₄ alkyl, C₁-C₄ haloalkyl, C₃-C₆ cycloalkyl, C₃-C₆ halocycloalkyl, —O(C₁-C₄ alkyl), —O(C₁-C₄ haloalkyl) or halogen;
R²⁰ is a bond, —NH—, —NMe-, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

A is a phenyl or 5- or 6-membered heteroaryl group, wherein A is substituted in the a position with B, in the β position with R⁷ and in the α′ position with R⁴ (relative to the point of attachment of A to R¹—S(X)(O)—NH—CY—NH—), and wherein A is optionally further substituted. In one embodiment, A is phenyl or a 5- or 6-membered heteroaryl group comprising one, two or three nitrogen and/or oxygen and/or sulfur ring atoms, wherein A is substituted in the a position with B, in the β position with R⁷ and in the α′ position with R⁴, and wherein A is optionally further substituted. In one embodiment, A is phenyl or a 5- or 6-membered heteroaryl group comprising one or two nitrogen and/or oxygen ring atoms, wherein A is substituted in the a position with B, in the β position with R⁷ and in the α′ position with R⁴, and wherein A is optionally further substituted. In one embodiment, A is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl or isothiazolyl group, wherein A is substituted in the a position with B, in the β position with R⁷ and in the α′ position with R⁴, and wherein A is optionally further substituted. In one embodiment, A is a phenyl, pyrimidinyl, pyrazolyl or imidazolyl group, wherein A is substituted in the a position with B, in the β position with R⁷ and in the α′ position with R⁴, and wherein A is optionally further substituted. In one embodiment, A is a phenyl or imidazolyl group, wherein A is substituted in the a position with B, in the β position with R⁷ and in the α′ position with R⁴, and wherein A is optionally further substituted.

A is optionally further substituted. In one embodiment, A is substituted in the γ position (relative to the point of attachment of A to R¹—S(X)(O)—NH—CY—NH—) with halogen or cyano. In one embodiment, A is substituted in the γ position with fluoro, chloro or cyano. In one embodiment, A is substituted in the γ position with fluoro.

B is a phenyl, 5- or 6-membered heteroaryl, or 4- to 6-membered saturated heterocyclic group, wherein B is optionally substituted. In one embodiment, B is a phenyl group, or a 5- or 6-membered heteroaryl group comprising one, two or three nitrogen and/or oxygen and/or sulfur ring atoms, or a 4- to 6-membered saturated heterocyclic group comprising one or two nitrogen and/or oxygen and/or sulfur ring atoms, wherein B is optionally substituted. In one embodiment, B is a phenyl group, or a 5- or 6-membered heteroaryl group comprising one or two nitrogen and/or oxygen ring atoms, or a 4- to 6-membered saturated heterocyclic group comprising one nitrogen or oxygen ring atom, wherein B is optionally substituted. In one embodiment, B is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, pyrrolidinyl, piperidinyl, tetrahydrofuranyl or tetrahydropyranyl group, wherein B is optionally substituted. In one embodiment, B is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, wherein B is optionally substituted. In one embodiment, B is a phenyl, pyridinyl, pyrimidinyl, pyrazolyl, imidazolyl, isoxazolyl or thiazolyl group, wherein B is optionally substituted. In one embodiment, B is a phenyl, pyridinyl, pyrimidinyl or pyrazolyl group, wherein B is optionally substituted. In one embodiment, B is a phenyl or pyridinyl group, wherein B is optionally substituted. In one embodiment, B is a pyridinyl group which is optionally substituted. In one embodiment, B is a pyridin-4-yl group which is optionally substituted.

B is optionally substituted. In one embodiment, B is optionally substituted with R² and optionally further substituted. In one embodiment, R² is hydrogen, halo, cyano, C₁-C₄ alkyl, C₁-C₄ haloalkyl, —R⁸—OH, -R⁸—O(C₁-C₄ alkyl), —R⁸—O(C₁-C₄ haloalkyl), —O—R¹⁰—OH, —O—R¹⁰—O(C₁-C₄ alkyl), —O—R¹⁰—O(C₁-C₄ haloalkyl), —R⁸—NH₂, —R⁸—NH(C₁-C₄ alkyl), —R⁸—NH(C₁-C₄ haloalkyl), —R⁸—N(C₁-C₄ alkyl)₂, —R⁸—N(C₁-C₄ alkyl)(C₁-C₄ haloalkyl), —R⁸—N(C₁-C₄ haloalkyl)₂, —R¹¹, —OR¹¹ or —O—R¹⁰—R¹¹; wherein
R⁸ is a bond, C₁-C₄ alkylene or C₁-C₄ haloalkylene;
R¹⁰ is C₁-C₄ alkylene or C₁-C₄ haloalkylene; and
R¹¹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from cyano, C₁-C₄ alkyl, C₁-C₄ haloalkyl, C₃-C₄ cycloalkyl, C₂-C₄ alkenyl, C₂-C₄ haloalkenyl, phenyl, benzyl, —OH, —O(C₁-C₄ alkyl), —O(C₁-C₄ haloalkyl), —NH₂, —NH(C₁-C₄ alkyl), —NH(C₁-C₄ haloalkyl), —N(C₁-C₄ alkyl)₂, —N(C₁-C₄ alkyl)(C₁-C₄ haloalkyl) and —N(C₁-C₄ haloalkyl) ₂.

In one embodiment, R² is hydrogen, halo, cyano, C₁-C₄ alkyl, C₁-C₄ haloalkyl, —R⁸—OH, —R⁸—O(C₁-C₄ alkyl), —R⁸—O(C₁-C₄ haloalkyl), —O—R¹⁰—OH, —O—R¹⁰—O(C₁-C₄ alkyl), —O—R¹⁰—O(C₁-C₄ haloalkyl), —R⁸—NH₂, —R⁸—NH(C₁-C₄ alkyl), —R⁸—NH(C₁-C₄ haloalkyl), —R⁸—N(C₁-C₄ alkyl)₂, —R⁸—N(C₁-C₄ alkyl)(C₁-C₄ haloalkyl), —R⁸—N(C₁-C₄ haloalkyl)₂, —RH, —OR¹¹ or —O—R¹⁰—R¹¹; wherein
R⁸ is a bond, C₁-C₄ alkylene or C₁-C₄ haloalkylene;
R¹⁰ is C₁-C₄ alkylene or C₁-C₄ haloalkylene; and
R¹¹ is C₃-C₆ cycloalkyl or a 4- to 6-membered saturated heterocyclic group comprising one or two nitrogen and/or oxygen and/or sulfur ring atoms, wherein the cycloalkyl or heterocyclic group is optionally halo-substituted and/or optionally substituted with one, two or three substituents independently selected from cyano, C₁-C₄ alkyl, C₁-C₄ haloalkyl, C₃-C₄ cycloalkyl, C₂-C₄ alkenyl, C₂-C₄ haloalkenyl, phenyl, benzyl, —OH, —O(C₁-C₄ alkyl), —O(C₁-C₄ haloalkyl), —NH₂, —NH(C₁-C₄ alkyl), —NH(C₁-C₄ haloalkyl), —N(C₁-C₄ alkyl)₂, —N(C₁-C₄ alkyl)(C₁-C₄ haloalkyl) and —N(C₁-C₄ haloalkyl)₂.

In one embodiment, R² is hydrogen, halo, cyano, C₁-C₄ alkyl, C₁-C₄ haloalkyl, —R⁸—OH, —R⁸—O(C₁-C₄ alkyl), —R⁸—O(C₁-C₄ haloalkyl), —O—R¹⁰—OH, —O—R¹⁰—O(C₁-C₄ alkyl), —O—R¹⁰—O(C₁-C₄ haloalkyl), —R⁸—NH₂, —R⁸—NH(C₁-C₄ alkyl), —R⁸—NH(C₁-C₄ haloalkyl), —R⁸—N(C₁-C₄ alkyl)₂, —R⁸—N(C₁-C₄ alkyl)(C₁-C₄ haloalkyl), —R⁸-N(C₁-C₄ haloalkyl)₂, —R¹¹, —OR¹¹ or —O—R¹⁰—R¹¹; wherein
R⁸ is a bond, C₁-C₃ alkylene or C₁-C₃ haloalkylene;
R¹⁰ is C₁-C₃ alkylene or C₁-C₃ haloalkylene; and
R¹¹ is C₃-C₆ cycloalkyl or a 4- to 6-membered saturated heterocyclic group comprising one nitrogen or oxygen ring atom, wherein the cycloalkyl or heterocyclic group is optionally halo-substituted and/or optionally substituted with one, two or three substituents independently selected from cyano, C₁-C₄ alkyl, C₁-C₄ haloalkyl, C₃-C₄ cycloalkyl, C₂-C₄ alkenyl, C₂-C₄ haloalkenyl, phenyl, benzyl, —OH, —O(C₁-C₄ alkyl), —O(C₁-C₄ haloalkyl), —NH₂, —NH(C₁-C₄ alkyl), —NH(C₁-C₄ haloalkyl), —N(C₁-C₄ alkyl)₂, —N(C₁-C₄ alkyl)(C₁-C₄ haloalkyl) and —N(C₁-C₄ haloalkyl)₂.

In one embodiment, R² is hydrogen, halo, cyano, C₁-C₄ alkyl, C₁-C₄ haloalkyl, —R⁸—OH, —R^{8l —O(C}₁-C₄ alkyl), —R⁸—O(C₁-C₄ haloalkyl), —O—R¹⁰—OH, —O—R¹⁰—O(C₁-C₄ alkyl), —RH, —OR¹¹ or —O—R¹⁰—R¹¹; wherein
R⁸ is a bond or —CH₂—;
R¹⁰ is C₁-C₃ alkylene; and
R¹¹ is a cyclopropyl, cyclobutyl, cyclopentyl, cyclohexyl, pyrrolidinyl, piperidinyl, tetrahydrofuranyl or tetrahydropyranyl group, all optionally halo-substituted and/or optionally substituted with one or two substituents independently selected from C₁-C₃ alkyl, C₁-C₃ haloalkyl, C₂-C₃ alkenyl, C₂-C₃ haloalkenyl, phenyl, benzyl, —OH, —O(C₁-C₃ alkyl), —O(C₁-C₃ haloalkyl), —NH₂, —NH(C₁-C₃ alkyl), —NH(C₁-C₃ haloalkyl), —N(C₁-C₃ alkyl)₂, —N(C₁-C₃ alkyl)(C₁-C₃ haloalkyl) and —N(C₁-C₃ haloalkyl)₂.

In one embodiment, R² is hydrogen, halo, cyano, C₁-C₃ alkyl, C₁-C₃ haloalkyl, —R⁸—OH, —R⁸—O(C₁-C₃ alkyl), —R⁸—O(C₁-C₃ haloalkyl), —O—R¹⁰—OH, —O—R¹⁰—O(C₁-C₃ alkyl), —R¹¹, —OR¹¹ or —O—R¹⁰ —R¹¹; wherein
R⁸ is a bond or —CH₂—;
R¹⁰ is C₁-C₃ alkylene; and
R¹¹ is a cyclopropyl, cyclobutyl, cyclopentyl, cyclohexyl, pyrrolidinyl, piperidinyl, tetrahydrofuranyl or tetrahydropyranyl group, all optionally substituted with one or two substituents independently selected from fluoro, C₁-C₃ alkyl, C₂-C₃ alkenyl, phenyl, benzyl, —OH, —O(C₁-C₃ alkyl), —NH₂, —NH(C₁-C₃ alkyl) and —N(C₁-C₃ alkyl)₂.

In one embodiment, R² is hydrogen, halo, cyano, C₁-C₃ alkyl, C₁-C₃ haloalkyl, —R⁸—OH, —R⁸—O(C₁-C₃ alkyl), —R⁸—O(C₁-C₃ haloalkyl), —O—R¹⁰—OH, —O—R¹⁰—O(C₁-C₃ alkyl), —R¹¹, —OR¹¹ or —O—R¹⁰—R¹¹; wherein
R⁸ is a bond or —CH₂—;
R¹⁰ is C₁-C₃ alkylene; and
R¹¹ is a cyclopropyl, cyclobutyl, cyclopentyl, cyclohexyl, pyrrolidinyl, piperidinyl, tetrahydrofuranyl or tetrahydropyranyl group, all optionally substituted with one or two substituents independently selected from fluoro, methyl, —OH, —OMe, —NHMe and —NMe₂.

In one embodiment, when R¹¹ is a pyrrolidinyl or piperidinyl group, the pyrrolidinyl or piperidinyl group is substituted on the nitrogen ring atom.

B may be substituted with R² in the α, β or γ position (relative to the point of attachment of B to A). In one embodiment, B is substituted with R² in the β or γ position. In one embodiment, B is substituted with R² in the β position.

In one embodiment, B is a pyridin-4-yl group, substituted with R² in the β position, and optionally further substituted.

In one embodiment, B is optionally substituted with R² and optionally further substituted with one or two substituents independently selected from halo, C₁-C₃ alkyl, —O(C₁-C₃ alkyl), —OH, —NH₂ and —CN. In one embodiment, B is further substituted with one or two substituents independently selected from fluoro, chloro, methyl, ethyl, —OMe, —OEt, —OH, —NH₂ and —CN. In one embodiment, B is further substituted with methyl.

X is O, NH or N(CN). In one embodiment, X is O or NH. In one embodiment, X is O.

Y is O or S. In one embodiment, Y is O.

R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted; wherein R²⁰ is a bond, —NH—, —NMe—, C₁-C₄ alkylene or C₁-C₄ haloalkylene; R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³; R²² is a bond, C₁-C₄ alkylene or C₁-C₄ haloalkylene; and R²³ is a C₃-C₆ cycloalkyl or 4- to 6-membered saturated heterocyclic group, all optionally halo-substituted.

In one embodiment, R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted; wherein R²⁰ is a bond, C₁-C₄ alkylene or C₁-C₄ haloalkylene; R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³; R²² is a bond, C₁-C₄ alkylene or C₁-C₄ haloalkylene; and R²³ is a C₃-C6 cycloalkyl or 4- to 6-membered saturated heterocyclic group, all optionally halo-substituted.

In one embodiment, R¹ is a C₁-C₄ alkyl, C₂-C₄ alkenyl, —NH(C₁-C₃ alkyl), —N(C₁-C₃ alkyl)₂, or —R²⁰—R²¹ group, all optionally halo-substituted; wherein R²⁰ is a bond, C₁-C₃ alkylene or C₁-C₃ haloalkylene; R²¹ is a C₃-C₆ cycloalkyl or phenyl group, or a 4- to 6-membered saturated heterocyclic group comprising one or two nitrogen and/or oxygen and/or sulfur ring atoms, or a 5- or 6-membered heteroaryl group comprising one, two or three nitrogen and/or oxygen and/or sulfur ring atoms, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁—C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³; R²² is a bond, C₁-C₄ alkylene or C₁-C₄ haloalkylene; and R²³ is C₃-C₆ cycloalkyl or a 4- to 6-membered saturated heterocyclic group comprising one or two nitrogen and/or oxygen and/or sulfur ring atoms, all optionally halo-substituted.

In one embodiment, R¹ is a C₁-C₄ alkyl, C₂-C₄ alkenyl, —NHMe, —NMe₂, —NHEt, —NEt₂, —NMeEt or —R²⁰—R²¹ group, all optionally halo-substituted; wherein R²⁰ is a bond or C₁-C₂ alkylene; R²¹ is a C₃-C₆ cycloalkyl, phenyl, pyrrolidinyl, piperidinyl, tetrahydrofuranyl, tetrahydropyranyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, all optionally halo-substituted and/or optionally substituted with one or two substituents independently selected from cyano, C₁-C₄ alkyl, —R²²—OH, —R²²—O(C₁-C₄ alkyl), —R²²—NH(C₁-C₄ alkyl), —R²²—N(C₁-C₄ alkyl)₂ and —R²²—R²³; R²² is a bond or C₁-C₄ alkylene; and R²³ is a C₃-C₆ cycloalkyl, azetidinyl, pyrrolidinyl, piperidinyl, tetrahydrofuranyl or tetrahydropyranyl group.

In one embodiment, R¹ is C₁-C₄ alkyl, C₂-C₄ alkenyl, —NHMe, —NMe₂, —NHEt, —NEt₂ or —NMeEt, all optionally halo-substituted; or R¹ is a C₃-C₆ cycloalkyl, phenyl, pyrrolidinyl, piperidinyl, tetrahydrofuranyl, tetrahydropyranyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, all optionally halo-substituted and/or optionally substituted with one or two substituents independently selected from cyano, C₁-C₄ alkyl, —R²²—OH, —R²²—O(C₁-C₄ alkyl), —R²²—NH(C₁-C₄ alkyl), —R²²—N(C₁-C₄ alkyl)₂ and —R²²—R²³; wherein R²² is a bond or C₁-C₄ alkylene; and R²³ is a C₃-C₆ cycloalkyl, azetidinyl, pyrrolidinyl, piperidinyl, tetrahydrofuranyl or tetrahydropyranyl group.

In one embodiment, R¹ is C₁-C₄ alkyl, C₂-C₄ alkenyl, —NHMe, —NMe₂, —NHEt, —NEt₂ or —NMeEt, all optionally halo-substituted; or R¹ is a C₃-C₆ cycloalkyl, phenyl, pyrrolidinyl, piperidinyl, tetrahydrofuranyl, tetrahydropyranyl, furanyl, thiophenyl, pyrazolyl or imidazolyl group, all optionally halo-substituted and/or optionally substituted with one or two substituents independently selected from C₁-C₃ alkyl, —R²²—OH, —R²²—O(C₁-C₃ alkyl), —R²²—NH(C₁-C₃ alkyl), —R²²—N(C₁-C₃ alkyl)₂ and —R²²—R²³; wherein R²² is a bond or C₁-C₄ alkylene; and R²³ is a C₃-C₆ cycloalkyl, azetidinyl, pyrrolidinyl, piperidinyl, tetrahydrofuranyl or tetrahydropyranyl group.

In one embodiment, R¹ is C₁-C₄ alkyl, C₂-C₄ alkenyl, —NHMe, —NMe₂, —NHEt, —NEt₂ or —NMeEt; or R¹ is a C₃-C₆ cycloalkyl, phenyl, pyrrolidinyl, piperidinyl, tetrahydrofuranyl, tetrahydropyranyl, furanyl, thiophenyl, pyrazolyl or imidazolyl group, all optionally substituted with C₁-C₃ alkyl, —R²²—OH, —R²²—O(C₁-C₃ alkyl), —R²²—NH(C₁-C₃ alkyl), —R²²—N(C₁-C₃ alkyl)₂ or —R²²—R²³; wherein R²² is a bond or C₁-C₄ alkylene; and R²³ is a C₃-C₆ cycloalkyl, azetidinyl, pyrrolidinyl, piperidinyl, tetrahydrofuranyl or tetrahydropyranyl group.

In one embodiment, R¹ is C₁-C₄ alkyl, C₂-C₄ alkenyl, —NHMe, —NMe₂, —NHEt, —NEt₂ or —NMeEt; or R¹ is a cyclopropyl, cyclobutyl, cyclopentyl, cyclohexyl, phenyl, pyrrolidinyl, piperidinyl, tetrahydrofuranyl, tetrahydropyranyl, furanyl, thiophenyl, pyrazolyl or imidazolyl group, all optionally substituted with C₁-C₃ alkyl, —R²²—OH, —R²²—O(C₁-C₃ alkyl), —R²²—NH(C₁-C₃ alkyl), —R²²—N(C₁-C₃ alkyl)₂ or —R²²—R²³; wherein R²² is a bond or C₁-C₄ alkylene; and R²³ is a cyclopropyl, cyclobutyl, cyclopentyl, cyclohexyl, azetidinyl, pyrrolidinyl, piperidinyl, tetrahydrofuranyl or tetrahydropyranyl group.

In one embodiment, R¹ is methyl, ethyl or —NMe₂; or R¹ is a cyclopropyl, phenyl, furanyl or pyrazolyl group, all optionally substituted with methyl, ethyl, isopropyl, CMe₂(OH) or cyclopropyl.

In one embodiment, when R¹ is a pyrrolidinyl, piperidinyl, pyrazolyl or imidazolyl group, the pyrrolidinyl, piperidinyl, pyrazolyl or imidazolyl group is substituted on the nitrogen ring atom.

In one embodiment, A is a phenyl group, substituted in the a position with B, substituted in the β position with R⁷, substituted in the α′ position with R⁴, and optionally further substituted; and R¹ is methyl, ethyl or —NMe₂, or R¹ is a cyclopropyl, phenyl, furanyl or pyrazolyl group, all optionally substituted with methyl, ethyl, isopropyl, CMe₂(OH) or cyclopropyl.

In another embodiment, A is an imidazolyl group, substituted in the a position with B, substituted in the β position with R⁷, substituted in the α′ position with R⁴, and optionally further substituted; and R¹ is a furanyl or pyrazolyl group, both optionally io substituted with methyl, ethyl, isopropyl, CMe₂(OH) or cyclopropyl.

In one embodiment, R⁴ is monovalent, and attached to A in the α′ position (relative to the point of attachment of A to R¹—S(X)(O)—NH—CY-NH—), and selected from C₁-C₄ alkyl, C₃-C₆ cycloalkyl and phenyl, all optionally halo-substituted and/or optionally is substituted with one or two substituents independently selected from oxo (═O), —OH, —O(C₁-C₄ alkyl) and —O(C₁-C₄ haloalkyl). In one embodiment, R⁴ is monovalent, and attached to A in the α′ position, and selected from methyl, ethyl, n-propyl, isopropyl, n-butyl, sec-butyl, isobutyl, t-butyl, cyclopropyl, cyclobutyl, cyclopentyl, cyclohexyl and phenyl, all optionally halo-substituted and/or optionally substituted with one substituent selected from oxo, —OH, —O(C₁-C₄ alkyl) and —O(C₁-C₄ haloalkyl). In one embodiment, R⁴ is monovalent, and attached to A in the α′ position, and selected from isopropyl, sec-butyl, isobutyl, t-butyl, cyclopropyl, cyclobutyl, cyclopentyl, cyclohexyl and phenyl, all optionally halo-substituted. In one embodiment, R⁴ is monovalent, and attached to A in the α′ position, and selected from isopropyl, cyclopentyl, cyclohexyl and phenyl, all optionally halo-substituted. In one embodiment, R⁴ is monovalent, and attached to A in the α′ position, and selected from isopropyl, cyclopentyl, cyclohexyl and phenyl. In one embodiment, R⁴ is an isopropyl group attached to A in the α′ position.

3o In an alternative embodiment, R⁴ is divalent, and attached to A in the α′ and β′ positions (relative to the point of attachment of A to R¹—S(X)(O)—NH—CY—NH—), and selected from —CH₂CH₂CH₂—, —CH═CHCH₂—, —CH₂CH═CH—, —CH₂CH₂₀—, —OCH₂CH₂—, —CH₂CH₂CH₂CH₂—and —CH═CH-CH═CH—, all optionally halo-substituted and/or optionally substituted with one or two substituents independently selected from oxo (═O), —OH, —O(C₁-C₄ alkyl) and —O(C₁-C₄ haloalkyl). In one embodiment, R⁴ is divalent, and attached to A in the α′ and β′ positions, and selected from —CH₂CH₂CH₂—, —CH₂CH₂O—and —OCH₂CH₂—, all optionally halo-substituted and/or optionally substituted with one substituent selected from oxo, —OH, —O(C₁-C₄ alkyl) and —O(C₁-C₄ haloalkyl). In one embodiment, R⁴ is divalent, and attached to A in the α′ and β′ positions, and selected from —CH₂CH₂CH₂—, —CH₂CH₂O— and —OCH₂CH₂—, all optionally halo-substituted. In one embodiment, R⁴ is divalent, and attached to A in the α′ and β′ positions, and selected from —CH₂CH₂CH₂—, —CH₂CH₂O— and —OCH₂CH₂—. In one embodiment, R⁴ is a —CH₂CH₂CH₂—group attached to A in the α′ and β′ positions.

R⁷ is C₁-C₄ alkyl, C₁-C₄ haloalkyl, C₃-C₆ cycloalkyl, C₃-C₆ halocycloalkyl, —O(C₁-C₄ alkyl), —O(C₁-C₄ haloalkyl) or halogen. In one embodiment, R⁷ is C₁-C₄ alkyl, C₁-C₄ haloalkyl, C₃-C₆ cycloalkyl, C₃-C₆ halocycloalkyl or halogen. In one embodiment, R⁷ is methyl, ethyl, halomethyl, haloethyl, cyclopropyl, halocyclopropyl or halogen. In one embodiment, R⁷ is methyl, ethyl, trifluoromethyl, cyclopropyl or fluoro. In one embodiment, R⁷ is methyl, ethyl, cyclopropyl or fluoro. In one embodiment, R⁷ is methyl.

The first aspect of the invention also provides a compound of formula (IA):

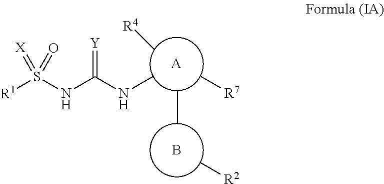

wherein:
A is a phenyl or 5- or 6-membered heteroaryl group, wherein A is substituted in the a position with B, in the β position with R⁷ and in the α′ position with R⁴, and wherein A is optionally further substituted;
B is a phenyl, 5- or 6-membered heteroaryl, or 4- to 6-membered saturated heterocyclic group, wherein B is substituted with R², and wherein B is optionally further substituted;
X is O, NH or N(CN);
Y is O or S;
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R² is hydrogen, halo, cyano, C₁-C₄ alkyl, C₁-C₄ haloalkyl, —R⁸—OH, —R⁸—O(C₁-C₄ alkyl), —R⁸—O(C₁-C₄ haloalkyl), —O—R¹⁰—OH, —O—R¹⁰—O(C₁-C₄ alkyl), —O—R¹⁰—O(C₁-C₄ haloalkyl), —R⁸—NH₂, —R⁸—NH(C₁-C₄ alkyl), —R⁸—NH(C₁-C₄ haloalkyl), —R⁸—N(C₁-C₄ alkyl)₂, —R⁸—N(C₁-C₄ alkyl)(C₁-C₄ haloalkyl), —R⁸—N(C₁-C₄ haloalkyl)₂, —R¹¹, —OR¹¹ or —O—R¹⁰—R¹¹;
either R⁴ is monovalent, and attached to A in the α′ position, and selected from C₁-C₄ alkyl, C₃-C₆ cycloalkyl and phenyl, all optionally halo-substituted and/or optionally substituted with one or two substituents independently selected from oxo, —OH, —O(C₁-C₄ alkyl) and —O(C₁-C₄ haloalkyl);
or R⁴ is divalent, and attached to A in the α′ and β′ positions, and selected from —CH₂CH₂CH₂—, —CH═CHCH₂—, —CH₂CH═CH—, —CH₂CH₂₀—, —OCH₂CH₂—, —CH₂CH₂CH₂CH₂— and —CH═CH—CH═CH—, all optionally halo-substituted and/or optionally substituted with one or two substituents independently selected from oxo, —OH, —O(C₁-C₄ alkyl) and —O(C₁-C₄ haloalkyl);
R⁷ is C₁-C₄ alkyl, C₁-C₄ haloalkyl, C₃-C₆ cycloalkyl, C₃-C₆ halocycloalkyl, —O(C₁-C₄ alkyl), —O(C₁-C₄ haloalkyl) or halogen;
R⁸ is a bond, C₁-C₄ alkylene or C₁-C₄ haloalkylene;
R¹⁰ is C₁-C₄ alkylene or C₁-C₄ haloalkylene;
R¹¹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from cyano, C₁-C₄ alkyl, C₁-C₄ haloalkyl, C₃-C₄ cycloalkyl, C₂-C₄ alkenyl, C₂-C₄ haloalkenyl, phenyl, benzyl, —OH, —O(C₁-C₄ alkyl), —O(C₁-C₄ haloalkyl), —NH₂, —NH(C₁-C₄ alkyl), —NH(C₁-C₄ haloalkyl), —N(C₁-C₄ alkyl)₂, —N(C₁-C₄ alkyl)(C₁-C₄ haloalkyl) and —N(C₁-C₄ haloalkyl)₂;
R²⁰ is a bond, —NH—, —NMe—, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

The embodiments of A, B, X, Y, R¹, R², R⁴, R⁷, R⁸, R¹⁰, R^{11l}, R²⁰, R²¹, R²² and R²³ described above in relation to the compounds of formula (I) apply equally to the compounds of formula (IA).

The first aspect of the invention also provides a compound of formula (II):

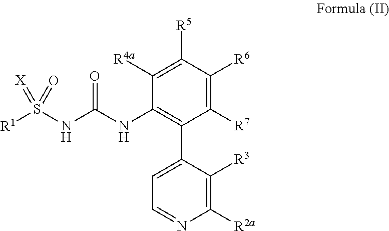

wherein:
X is O, NH or N(CN);
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R^{2a} is hydrogen, halo, cyano, C₁-C₄ alkyl, C₁-C₄ haloalkyl, —O(C₁-C₄ alkyl), —O(C₁-C₄ haloalkyl), —O-(alkoxyalkyl), —OR⁹ or —OCH₂—R⁹;
R³ is hydrogen or methyl;
R^{4a} is C₁-C₄ alkyl, C₃-C₆ cycloalkyl or phenyl, all optionally halo-substituted;
R⁵ is hydrogen; or
R^{4a} and R⁵ together form —CH₂CH₂CH₂—, —CH₂CH₂O—or —OCH₂CH₂—, all optionally halo-substituted;
R⁶ is hydrogen, halogen or cyano;
R⁷ is C₁-C₄ alkyl, C₁-C₄ haloalkyl, C₃-C₆ cycloalkyl, C₃-C₆ halocycloalkyl or halogen;
R⁹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from C₁-C₄ alkyl, C₂-C₄ alkenyl, phenyl, benzyl, —OH, —O(C₁-C₄ alkyl), —NH(C₁-C₄ alkyl) and —N(C₁-C₄ alkyl)₂;
R²⁰ is a bond, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

The embodiments of X, R¹, R⁷, R²⁰, R²¹, R²² and R²³ described above in relation to the compounds of formula (I) apply equally to the compounds of formula (II).

R^{2a} is hydrogen, halo, cyano, C₁-C₄ alkyl, C₁-C₄ haloalkyl, —O(C₁-C₄ alkyl), —O(C₁-C₄ haloalkyl), —O-(alkoxyalkyl), —OR⁹ or —OCH₂—R⁹; wherein R⁹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from C₁-C₄ alkyl, C₂-C₄ alkenyl, phenyl, benzyl, —OH, —O(C₁-C₄ alkyl), —NH(C₁-C₄ alkyl) and —N(C₁-C₄ alkyl)₂.

In one embodiment, R^{2a} is hydrogen, halo, cyano, C₁-C₄ alkyl, C₁-C₄ haloalkyl, —O(C₁-C₄ alkyl), —O(C₁-C₄ haloalkyl), —O-(alkoxyalkyl), —OR⁹ or —OCH₂—R⁹; wherein R⁹ is C₃-C₆ cycloalkyl or a 4- to 6-membered saturated heterocyclic group comprising one or two nitrogen and/or oxygen ring atoms, wherein the cycloalkyl or heterocyclic group is optionally halo-substituted and/or optionally substituted with one, two or three substituents independently selected from C₁-C₄ alkyl, C₂-C₄ alkenyl, phenyl, benzyl, —OH, —O(C₁-C₄ alkyl), —NH(C₁-C₄ alkyl) and —N(C₁-C₄ alkyl)₂.

In one embodiment, R^{2a} is hydrogen, cyano, C₁-C₃ alkyl, C₁-C₃ haloalkyl, —O(C₁-C₃ alkyl), —O(C₁-C₃ haloalkyl), —O-(alkoxyalkyl), —OR⁹ or —OCH₂—R⁹; wherein R⁹ is C₃-C₆ cycloalkyl or a 4- to 6-membered saturated heterocyclic group comprising one nitrogen or oxygen ring atom, wherein the cycloalkyl or heterocyclic group is optionally halo-substituted and/or optionally substituted with one or two substituents independently selected from C₁-C₃ alkyl, —OH, —O(C₁-C₃ alkyl), —NH(C₁-C₃ alkyl) and —N(C₁-C₃ alkyl)₂.

In one embodiment, R^{2a} is hydrogen, cyano, C₁-C₃ alkyl, C₁-C₃ haloalkyl, —O(C₁-C₃ alkyl), —O(C₁-C₃ haloalkyl), —O-(alkoxyalkyl), —OR⁹ or —OCH₂—R⁹; wherein R⁹ is a C₃-C₆ cycloalkyl, pyrrolidinyl, piperidinyl, tetrahydrofuranyl or tetrahydropyranyl group, all optionally halo-substituted and/or optionally substituted with one or two substituents independently selected from C₁-C₃ alkyl, —OH, —O(C₁-C₃ alkyl), —NH(C₁-C₃ alkyl) and —N(C₁-C₃ alkyl)₂.

In one embodiment, R^{2a} is hydrogen, cyano, methyl, ethyl, n-propyl, isopropyl, halomethyl, haloethyl, —OMe, —OEt, —O-(halomethyl), —O-(haloethyl), —O-(methoxyalkyl) or —OR⁹; wherein R⁹ is a cyclopropyl, cyclobutyl, cyclopentyl, cyclohexyl, pyrrolidinyl, piperidinyl, tetrahydrofuranyl or tetrahydropyranyl group, all optionally substituted with one substituent selected from methyl, ethyl, —OH, —OMe, —OEt, —NHMe, —NMe₂, —NHEt, —NEt₂ and —NMeEt.

In one embodiment, when R⁹ is a pyrrolidinyl or piperidinyl group, the pyrrolidinyl or piperidinyl group is substituted on the nitrogen ring atom.

R³ is hydrogen or methyl. In one embodiment, R³ is hydrogen. In one embodiment, R³ is methyl.

In one embodiment of the compounds of formula (II), R⁵ is hydrogen and R^{4a} is C₁-C₄ alkyl, C₃-C₆ cycloalkyl or phenyl, all optionally halo-substituted. In one embodiment, R^{4a} is methyl, ethyl, n-propyl, isopropyl, n-butyl, sec-butyl, isobutyl, t-butyl, cyclopropyl, cyclobutyl, cyclopentyl, cyclohexyl or phenyl, all optionally halo-substituted. In one embodiment, R^{4a} is isopropyl, sec-butyl, isobutyl, t-butyl, cyclopropyl, cyclobutyl, cyclopentyl, cyclohexyl or phenyl, all optionally halo-substituted. In one embodiment, R^{4a} is isopropyl, cyclopentyl, cyclohexyl or phenyl, all optionally halo-substituted. In one embodiment, R^{4a} is isopropyl, cyclopentyl, cyclohexyl or phenyl. In one embodiment, R^{4a} is isopropyl.

In an alternative embodiment of the compounds of formula (II), R^{4a} and R⁵ together form —CH₂CH₂CH₂—, —CH₂CH₂O— or —OCH₂CH₂—, all optionally halo-substituted. In one embodiment, R^{4a} and R⁵ together form —CH₂CH₂CH₂—, —CH₂CH₂O— or —OCH₂CH₂—. In one embodiment, R^{4a} and R⁵ together form —CH₂CH₂CH₂—.

R⁶ is hydrogen, halogen or cyano. In one embodiment, R⁶ is hydrogen, fluoro, chloro or cyano. In one embodiment, R⁶ is hydrogen or fluoro.

In one embodiment, the present invention provides a compound of formula (II), wherein:
X is O or NH;
R¹ is C₁-C₄ alkyl, C₂-C₄ alkenyl, —NHMe, —NMe₂, —NHEt, —NEt₂ or —NMeEt, all optionally halo-substituted; or R¹ is a C₃-C₆ cycloalkyl, phenyl, pyrrolidinyl, piperidinyl, tetrahydrofuranyl, tetrahydropyranyl, furanyl, thiophenyl, pyrazolyl or imidazolyl group, all optionally halo-substituted and/or optionally substituted with one or two substituents independently selected from C₁-C₃ alkyl, —R²²—OH, —R²²—O(C₁-C₃ alkyl), —R²²—NH(C₁-C₃ alkyl), —R²²—N(C₁-C₃ alkyl)₂ and —R²²—R²³;
R^{2a} is hydrogen, cyano, C₁-C₃ alkyl, C₁-C₃ haloalkyl, —O(C₁-C₃ alkyl), —O(C₁-C₃ haloalkyl), —O-(alkoxyalkyl), —OR⁹ or —OCH₂—R⁹;
R³ is hydrogen or methyl;
R^{4a} is isopropyl, cyclopentyl, cyclohexyl or phenyl;
R⁵ is hydrogen; or
R^{4a} and R⁵ together form —CH₂CH₂CH₂—, —CH₂CH₂O— or —OCH₂CH₂—;
R⁶ is hydrogen, halogen or cyano;
R⁷ is methyl, ethyl, cyclopropyl or fluoro;
R⁹ is a C₃-C₆ cycloalkyl, pyrrolidinyl, piperidinyl, tetrahydrofuranyl or tetrahydropyranyl group, all optionally halo-substituted and/or optionally substituted with one or two substituents independently selected from C₁-C₃ alkyl, —OH, —O(C₁-C₃ alkyl), —NH(C₁-C₃ alkyl) and —N(C₁-C₃ alkyl)₂;
R²² is a bond or C₁-C₄ alkylene; and
R²³ is a C₃-C₆ cycloalkyl, azetidinyl, pyrrolidinyl, piperidinyl, tetrahydrofuranyl or tetrahydropyranyl group.

The first aspect of the invention also provides a compound of formula (III):

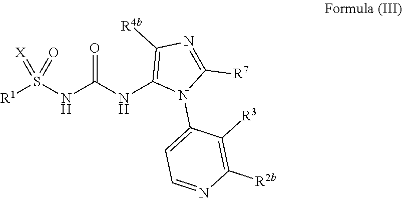

wherein:
X is O, NH or N(CN);
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R^{2b} is hydrogen, halo, cyano, C₁-C₄ alkyl, C₁-C₄ haloalkyl, —O(C₁-C₄ alkyl) or —O(C₁-C₄ haloalkyl);
R³ is hydrogen or methyl;
R^{4b} to is C₁-C₄ alkyl or C₁-C₄ haloalkyl;
R⁷ is C₁-C₄ alkyl, C₁-C₄ haloalkyl, C₃-C₆ cycloalkyl, C₃-C₆ halocycloalkyl or halogen;
R²⁰ is a bond, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

The embodiments of X, R¹, R⁷, R²⁰, R²¹, R²² and R²³ described above in relation to the compounds of formula (I) apply equally to the compounds of formula (III).

R^{2b} is hydrogen, halo, cyano, C₁-C₄ alkyl, C₁-C₄ haloalkyl, —O(C₁-C₄ alkyl) or —O(C₁-C₄ haloalkyl). In one embodiment, R^{2b} is hydrogen, cyano, C₁-C₃ alkyl, C₁-C₃ haloalkyl, —O(C₁-C₃ alkyl) or —O(C₁-C₃ haloalkyl). In one embodiment, R^{2b} is hydrogen, C₁-C₃ alkyl, —O(C₁-C₃ alkyl) or —O(C₁-C₃ haloalkyl). In one embodiment, R^{2b} is hydrogen, methyl, trifluoromethyl or —OMe. In one embodiment, R^{2b} is hydrogen or —OMe.

R³ is hydrogen or methyl. In one embodiment, R³ is hydrogen. In one embodiment, R³ is methyl.

R^{4b} is C₁-C₄ alkyl or C₁-C₄ haloalkyl. In one embodiment, R^{4b} is methyl, ethyl, n-propyl, isopropyl, n-butyl, sec-butyl, isobutyl or t-butyl, all optionally halo-substituted. In one embodiment, R^{4b} is isopropyl, sec-butyl, isobutyl or t-butyl, all optionally halo-substituted. In one embodiment, R^{4b} is isopropyl optionally halo-substituted. In one embodiment, R^{4b} is isopropyl.

In one embodiment, the present invention provides a compound of formula (III), wherein:
X is O or NH;
R¹ is C₁-C₄ alkyl, C₂-C₄ alkenyl, —NHMe, —NMe₂, —NHEt, —NEt₂ or —NMeEt, all optionally halo-substituted; or R¹ is a C₃-C₆ cycloalkyl, phenyl, pyrrolidinyl, piperidinyl, tetrahydrofuranyl, tetrahydropyranyl, furanyl, thiophenyl, pyrazolyl or imidazolyl group, all optionally halo-substituted and/or optionally substituted with one or two substituents independently selected from C₁-C₃ alkyl, —R²²—OH, —R²²—O(C₁-C₃ alkyl), —R²²—NH(C₁-C₃ alkyl), —R²²—N(C₁-C₃ alkyl)₂ and —R²²—R²³;

R^{2b} is hydrogen, cyano, C₁-C₃ alkyl, C₁-C₃ haloalkyl, —O(C₁-C₃ alkyl) or —O(C₁-C₃ haloalkyl);
R³ is hydrogen or methyl;
R^{4b} is C₁-C₄ alkyl or C₁-C₄ haloalkyl;
R⁷ is methyl, ethyl, cyclopropyl or fluoro;
R²² is a bond or C₁-C₄ alkylene; and
R²³ is a C₃-C₆ cycloalkyl, azetidinyl, pyrrolidinyl, piperidinyl, tetrahydrofuranyl or tetrahydropyranyl group.

In one aspect of any of the above embodiments, R¹ contains from 1 to 30 atoms other than hydrogen. More typically, R¹ contains from 1 to 25 atoms other than hydrogen. More typically, R¹ contains from 1 to 20 atoms other than hydrogen. More typically, R¹ contains from 1 to 16 atoms other than hydrogen.

In one aspect of any of the above embodiments, A, B, R⁴ and R⁷ together contain from 11 to 5o atoms other than hydrogen. More typically, A, B, R⁴ and R⁷ together contain from 12 to 45 atoms other than hydrogen. More typically, A, B, R⁴ and R⁷ together contain from 13 to 40 atoms other than hydrogen. Most typically, A, B, R⁴ and R⁷ together contain from 14 to 35 atoms other than hydrogen.

In one aspect of any of the above embodiments, the compound of formula (I), (IA), (II) or (III) has a molecular weight of from 250 to 2,000 Da. Typically, the compound of formula (I), (IA), (II) or (III) has a molecular weight of from 300 to 1,000 Da. Typically, the compound of formula (I), (IA), (II) or (III) has a molecular weight of from 310 to 800 Da. More typically, the compound of formula (I), (IA), (II) or (III) has a molecular weight of from 320 to 650 Da.

A second aspect of the invention provides a compound selected from the group consisting of:

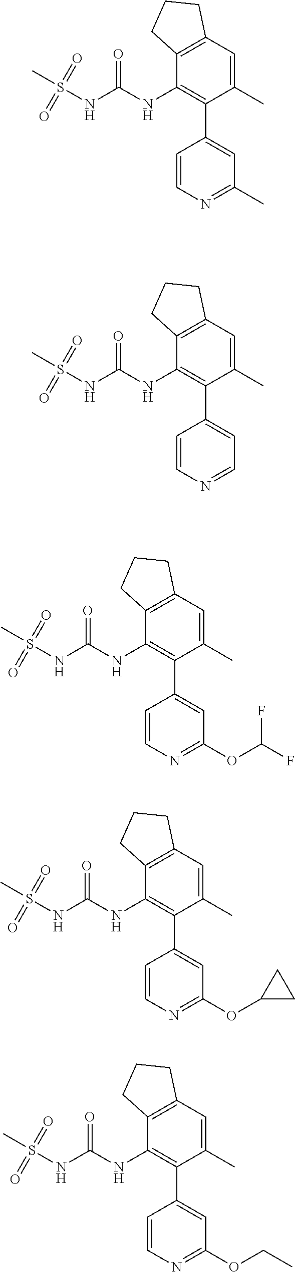

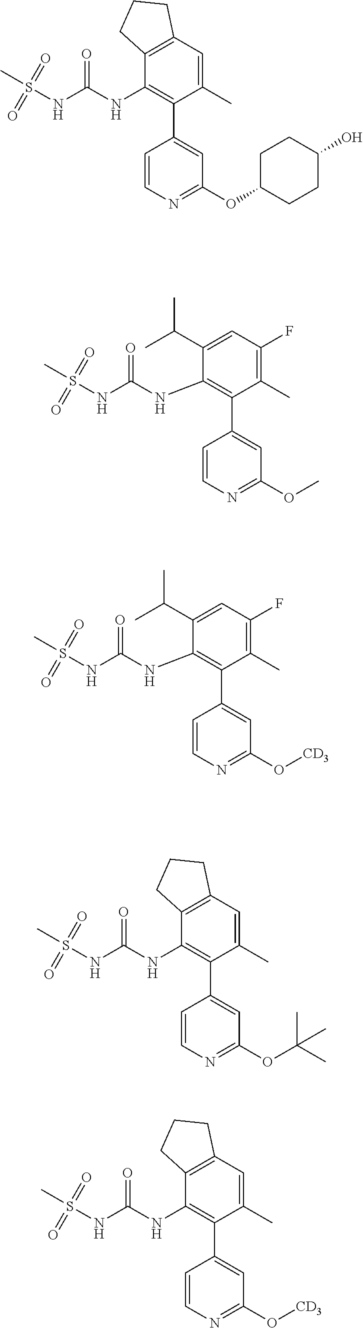

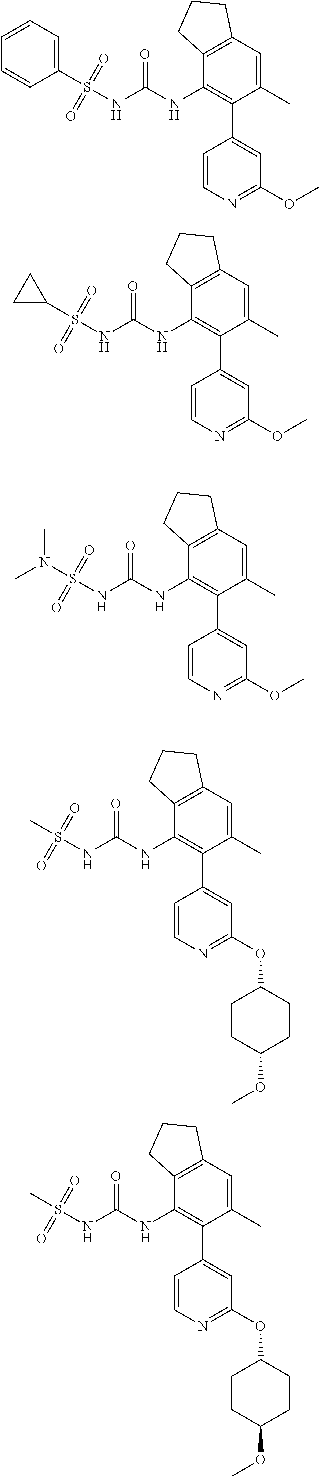

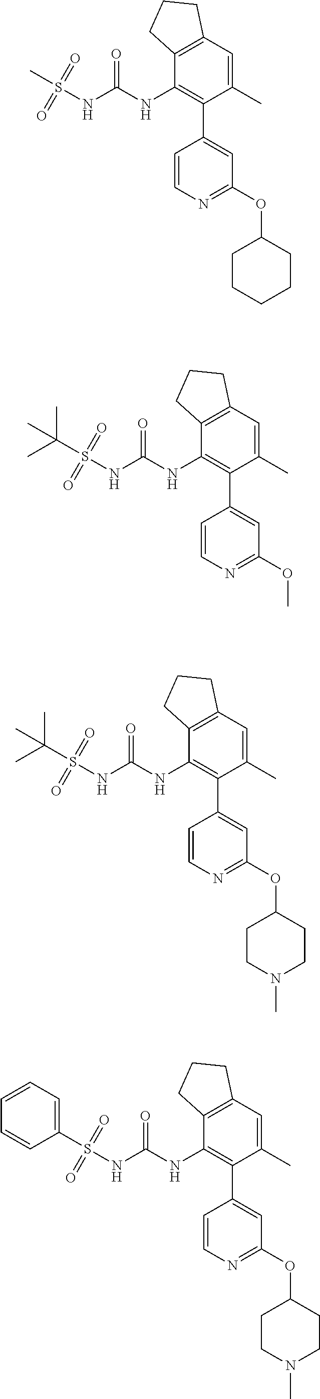

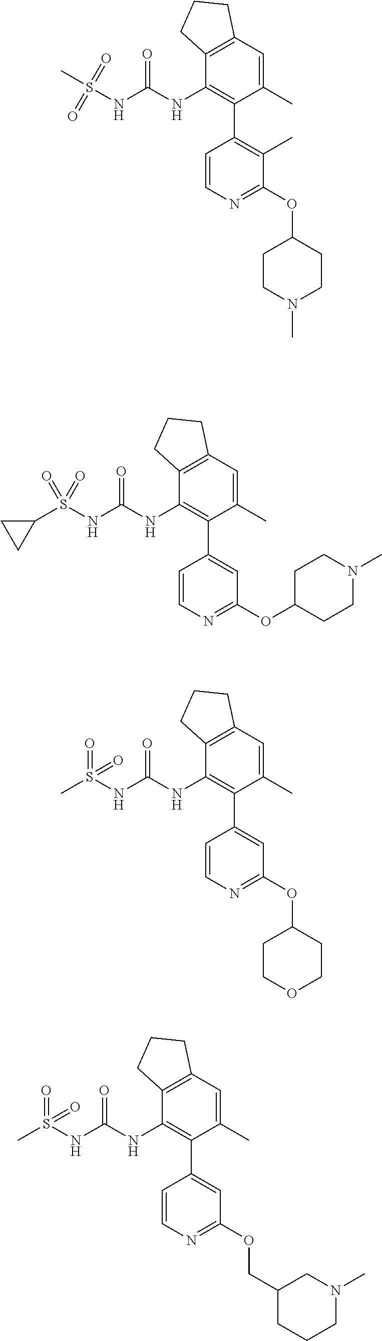

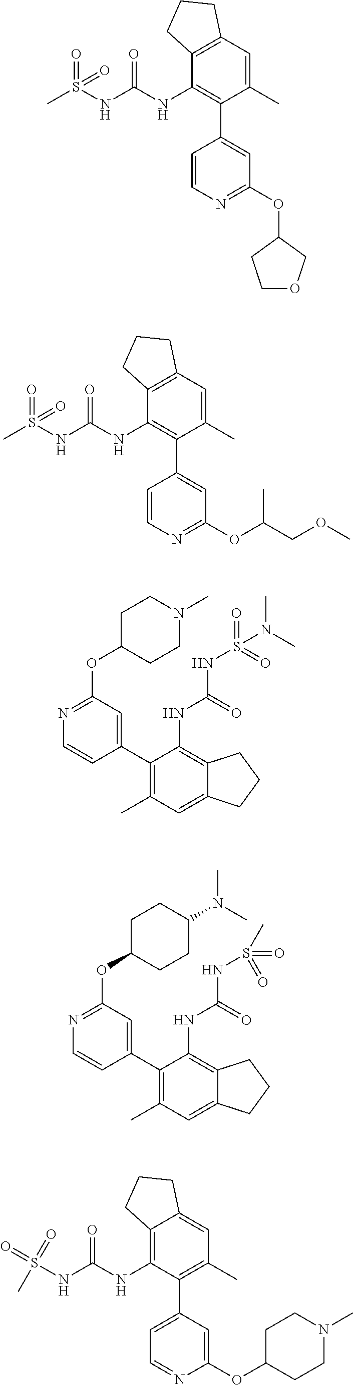

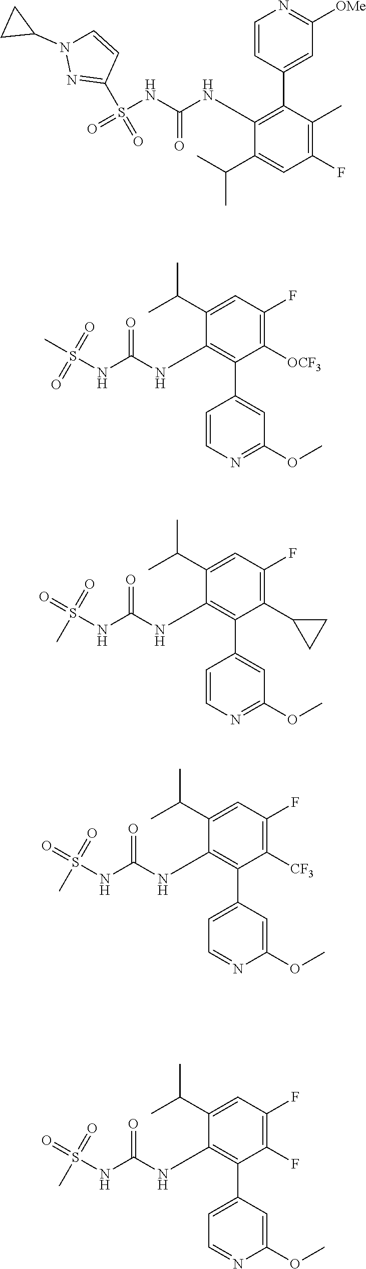

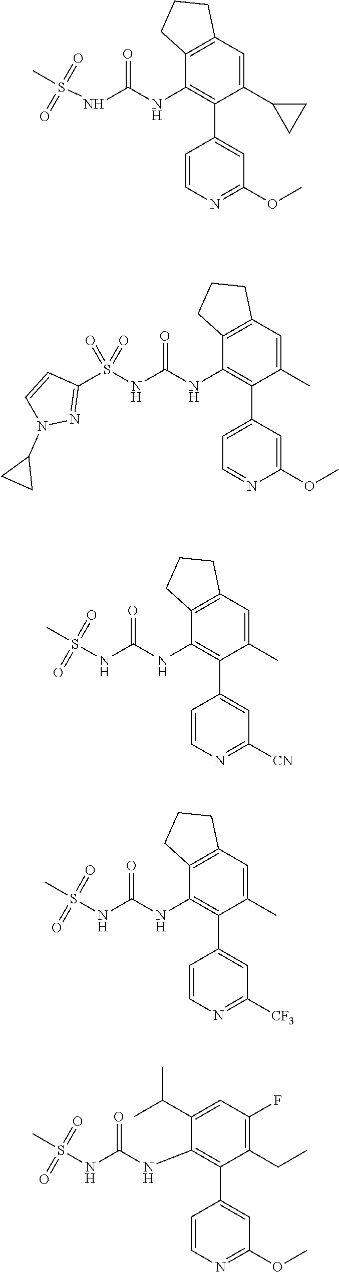

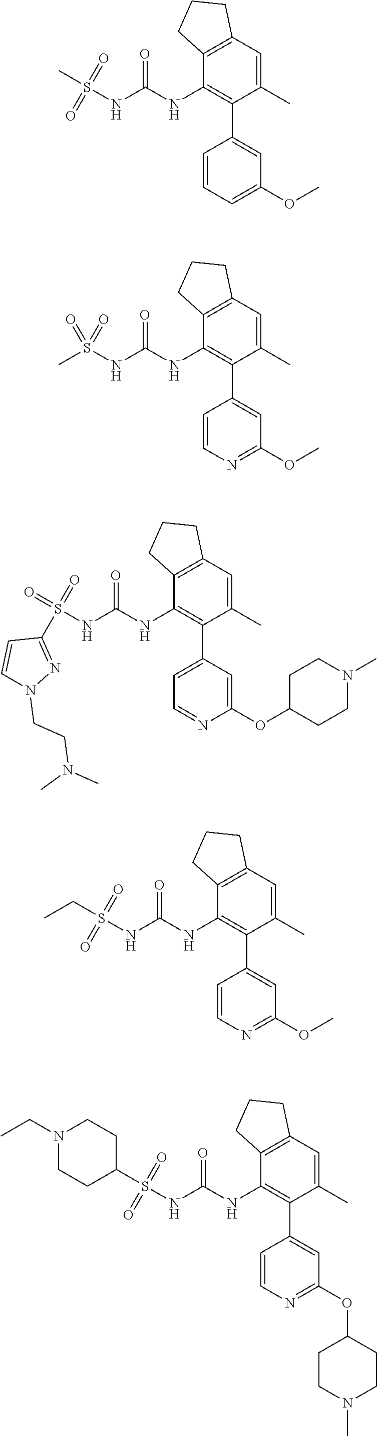

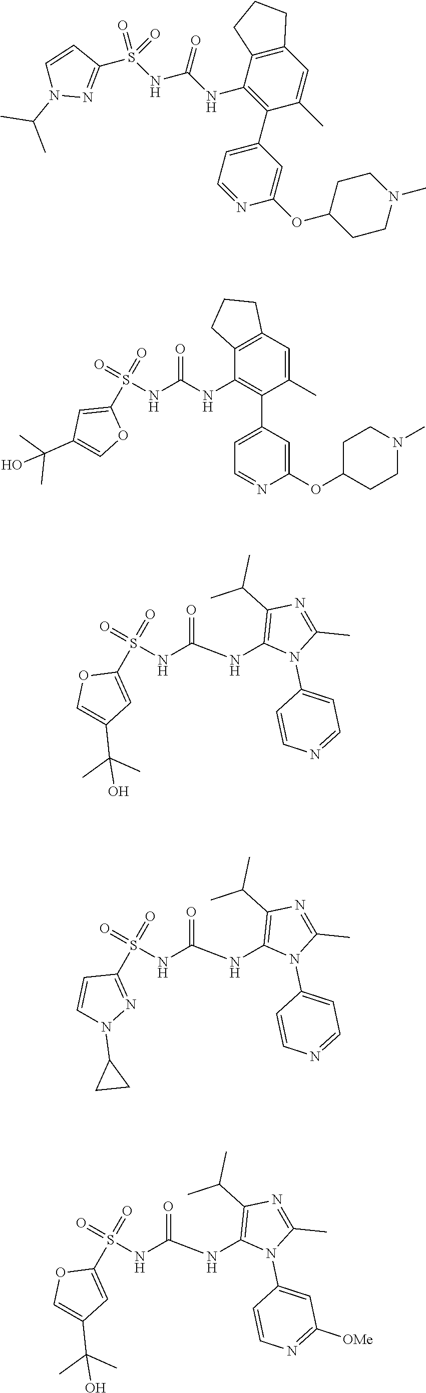 [SMILES: Cc1cc2c(c(NC(=O)NS(=O)(=O)c3ccn(C(C)C)n3)c1-c1ccnc(OC3CCN(C)CC3)c1)CCC2 ; Cc1cc2c(c(NC(=O)NS(=O)(=O)c3cc(C(C)(C)O)co3)c1-c1ccnc(OC3CCN(C)CC3)c1)CCC2 ; Cc1nc(C(C)C)c(NC(=O)NS(=O)(=O)c2cc(C(C)(C)O)co2)n1-c1ccncc1 ; Cc1nc(C(C)C)c(NC(=O)NS(=O)(=O)c2ccn(C3CC3)n2)n1-c1ccncc1 ; COc1cc(-n2c(C)nc(C(C)C)c2NC(=O)NS(=O)(=O)c2cc(C(C)(C)O)co2)ccn1]

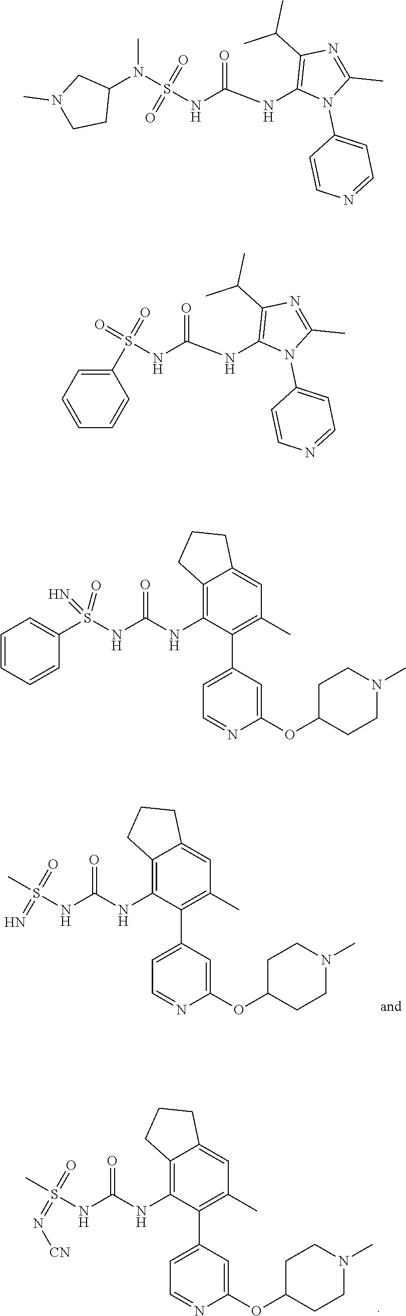

A third aspect of the invention provides a pharmaceutically acceptable salt, solvate or prodrug of any compound of the first or second aspect of the invention.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

Genetic diseases in which a role for NLRP3 has been suggested include sickle cell disease (Vogel et al., Blood, 130(Supp11): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur J Immunol, 40: 595-653, 2010). In particular, NLRP3 mutations have been found to be responsible for a set of rare autoinflammatory diseases known as CAPS (Ozaki et al., J Inflammation Research, 8: 15-27, 2015; Schroder et al., Cell, 140: 821-832, 2010; and Menu et al., Clinical and Experimental Immunology, 166: 1-15, 2011). CAPS are heritable diseases is characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of lung diseases including chronic obstructive pulmonary disorder (COPD), asthma (including steroid-resistant asthma and eosinophilic asthma), asbestosis, and silicosis (De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018), sepsis-associated encephalopathy (SAE) (Fu et al., Inflammation, 42(1): 306-318, 2019), postoperative cognitive dysfunction (POCD) (Fan et al., Front Cell Neurosci, 12: 426, 2018), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis, myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vasc Biol, 37(4): 694-706, 2017), cardiac injury induced by metabolic dysfunction (Pavillard et al., Oncotarget, 8(59): 99740-99756, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJMoa1707914, 2017).

Other diseases in which NLRP3 has been shown to be involved include:
ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);
liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure (Wang et al., Hepatol Res, 48(3): E194-E202, 2018);
kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), and renal hypertension (Krishnan et al., Br J Pharmacol, 173(4): 752-65, 2016);
conditions associated with diabetes including diabetic encephalopathy (Zhai et al., Molecules, 23(3): 522, 2018), diabetic retinopathy (Zhang et al., Cell Death Dis, 8(7): e2941, 2017), and diabetic hypoadiponectinemia (Zhang et al., Biochimica et Biophysica Acta (BBA) - Molecular Basis of Disease, 1863(6): 1556-1567, 2017);
inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);
inflammatory reactions in the joints (Braddock et al., Nat Rev Drug Disc, 3: 1-10, 2004) and osteoarthritis (Jin et al., PNAS, 108(36): 14867-14872, 2011);
amyotrophic lateral sclerosis (Gugliandolo et al., Inflammation, 41(1): 93-103, 2018);
cystic fibrosis (Iannitti et al., Nat Commun, 7: 10791, 2016);
stroke (Walsh et al., Nature Reviews, 15: 84-97, 2014);
chronic kidney disease (Granata et al., PLoS One, 10(3): e0122272, 2015);
Sjögren's syndrome (Vakrakou et al., Journal of Autoimmunity, 91: 23-33, 2018);
sickle cell disease (Vogel et al., Blood, 130(Suppl 1): 2234, 2017); and
colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), pain including multiple sclerosis-associated neuropathic pain (Khan et al., Inflammopharmacology, 26(1): 77-86, 2018), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus (Cohen et al., Cell Reports, 22(9): 2431-2441, 2018), bacillus cereus (Mathur et al., Nat Microbiol, 4: 362-374, 2019), salmonella typhimurium (Diamond et al., Sci Rep, 7(1): 6861, 2017), and group A streptococcus (LaRock et al., Science Immunology, 1(2): eaah 3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), leishmania (Novais et al., PLoS Pathogens, 13(2): e1006196, 2017), and plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012).

NLRP3 has also been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barr—syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections;

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, postoperative cognitive dysfunction, early brain injury, traumatic brain injury, and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure;

(x) renal diseases including chronic kidney disease, oxalate nephropathy, nephrocalcinosis, glomerulonephritis, diabetic nephropathy, kidney fibrosis including chronic crystal nephropathy, and renal hypertension;

(xi) ocular diseases including those of the ocular epithelium, age-related macular degeneration (AMD) (dry and wet), uveitis, corneal infection, diabetic retinopathy, optic nerve damage, dry eye, and glaucoma;

(xii) skin diseases including dermatitis such as contact dermatitis and atopic dermatitis, contact hypersensitivity, sunburn, skin lesions, hidradenitis suppurativa (HS), other cyst-causing skin diseases, and acne conglobata;

(xiii) lymphatic conditions such as lymphangitis and Castleman's disease;

(xiv) psychological disorders such as depression and psychological stress;

(xv) graft versus host disease;

(xvi) allodynia including mechanical allodynia;

(xvii) conditions associated with diabetes including diabetic encephalopathy, diabetic retinopathy, and diabetic hypoadiponectinemia; and

(xviii) any disease where an individual has been determined to carry a germline or somatic non-silent mutation in NLRP3.

In one embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxviii) transient ischemic attack;

(xxxiv) ulcerative colitis; or

(xxxv) Valosin Containing Protein disease.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis;

(iv) a gastrointestinal tract condition such as inflammatory bowel disease (including Crohn's disease and ulcerative colitis), colitis, gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, mycobacterium tuberculosis, mycobacterium avium intracellulare, pneumocystis carinii pneumonia, orchitis/epidydimitis, legionella, Lyme disease, influenza A, Epstein-Barr virus infection, viral encephalitis/aseptic meningitis, or pelvic inflammatory disease;

(xi) a renal condition such as mesangial proliferative glomerulonephritis, nephrotic syndrome, nephritis, glomerular nephritis, acute renal failure, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;

(xv) a cancer, including those cancers listed above;

(xvi) a burn, wound, trauma, haemorrhage or stroke;

(xvii) radiation exposure;

(xviii) obesity; and/or

(xix) pain such as inflammatory hyperalgesia.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

In some embodiments, the one or more chemotherapeutic agents are selected from abiraterone acetate, altretamine, amsacrine, anhydrovinblastine, auristatin, azathioprine, adriamycin, bexarotene, bicalutamide, BMS 184476, bleomycin, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, procarbazine, rhizoxin, sertenef, streptozocin, stramustine phosphate, tretinoin, tasonermin, taxol, topotecan, tamoxifen, teniposide, taxane, tegafur/uracil, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), interferon alpha, interferon beta, interferon gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), and/or cytokines (including interleukins, such as interleukin-2 (IL-2), or IL-10).

In some embodiments, the one or more antibodies may comprise one or more monoclonal antibodies. In some embodiments, the one or more antibodies are selected from abciximab, adalimumab, alemtuzumab, atlizumab, basiliximab, belimumab, bevacizumab, bretuximab vedotin, canakinumab, cetuximab, ceertolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumuab, ranibizumab, rituximab, tocilizumab, tositumomab, and/or trastuzumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from endostatin, angiogenin inhibitors, angiostatin, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides, such as cAMP, cGMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃).

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD 160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDRooi (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents, anti-inflammatory agents (including nonsteroidal anti-inflammatory agents), anti-psoriatic agents (including anthralin and its derivatives), vitamins and vitamin-derivatives (including retinoinds, and VDR receptor ligands), corticosteroids, ion channel blockers (including potassium channel blockers), immune system regulators (including cyclosporin, FK 506, and glucocorticoids), lutenizing hormone releasing hormone agonists (such as leuprolidine, goserelin, triptorelin, histrelin, bicalutamide, flutamide and/or nilutamide), and/or hormones (including estrogen).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

### Compound Synthesis

All solvents, reagents and compounds were purchased and used without further purification unless stated otherwise.

Abbreviations

2-MeTHF 2-methyltetrahydrofuran

Ac₂O acetic anhydride

AcOH acetic acid

aq aqueous

B₂Pin₂ bis(pinacolato)diboron, also called 4,4,4′,4′,5,5,5′,5′-octamethyl-2,2′- bi(1,3,2-dioxaborolane)

Boc tert-butyloxycarbonyl

br broad

Cbz carboxybenzyl

CDI 1,1-carbonyl-diimidazole

conc concentrated

d doublet

DABCO 1,4-diazabicyclo[2.2.2]octane

DCE 1,2-dichloroethane, also called ethylene dichloride

DCM dichloromethane

DIPEA N,N-diisopropylethylamine, also called Hiinig's base

DMA dimethylacetamide

DMAP 4-dimethylaminopyridine, also called N,N-dimethylpyridin-4-amine

DME dimethoxyethane

DMF N,N-dimethylformamide

DMSO dimethyl sulfoxide

EDC 1-ethyl-3-(3-dimethylaminopropyl)carbodiimide

eq or equiv equivalent

(ES⁺) electrospray ionization, positive mode

Et ethyl

EtOAc ethyl acetate

EtOH ethanol

h hour(s)

HATU 1-[bis(dimethylamino)methylene]-1H-1,2,3-triazolo[4,5-b]pyridinium 3-oxid hexafluorophosphate

HPLC high performance liquid chromatography

LC liquid chromatography

m multiplet

m-CPBA ₃-chloroperoxybenzoic acid

Me methyl

MeCN acetonitrile

MeOH methanol

(M+H)⁺ protonated molecular ion

MHz megahertz

min minute(s)

MS mass spectrometry

Ms mesyl, also called methanesulfonyl

MsCl mesyl chloride, also called methanesulfonyl chloride

MTBE methyl tert-butyl ether, also called tert-butyl methyl ether

m/z mass-to-charge ratio

NaOtBu sodium tert-butoxide

NBS 1-bromopyrrolidine-2,5-dione, also called N-bromosuccinimide

NCS 1-chloropyrrolidine-2,5-dione, also called N-chlorosuccinimide

NMP N-methylpyrrolidine

NMR nuclear magnetic resonance (spectroscopy)

Pd₂(dba)₃ tris(dibenzylideneacetone) dipalladium(o)

PdCl₂(dppf) [1,1′-bis(diphenylphosphino)ferrocene] dichloropalladium(II), also called Pd(dppf)Cl₂

PE petroleum ether

Ph phenyl

PMB p-methoxybenzyl, also called 4-methoxybenzyl

prep HPLC preparative high performance liquid chromatography

prep-TLC preparative thin layer chromatography

PTSA p-toluenesulfonic acid

q quartet

RP reversed phase

RT room temperature

s singlet

sat saturated

SCX solid supported cation exchange (resin)

sept septuplet

t triplet

T3P propylphosphonic anhydride

TBME tert-butyl methyl ether, also called methyl tert-butyl ether

TEA triethylamine

TFA 2,2,2-trifluoroacetic acid

THF tetrahydrofuran

TLC thin layer chromatography

wt % weight percent or percent by weight

Xphos 2-dicyclohexylphosphino-2′,4′,6′-triisopropylbiphenyl

Experimental Methods

Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker Avance III spectrometer at 400 MHz fitted with a BBO 5 mm liquid probe,
a Bruker 400 MHz spectrometer using ICON-NMR, under TopSpin program control,
a Bruker Avance III HD spectrometer at 500 MHz, equipped with a Bruker 5 mm SmartProbe™,
an Agilent VNMRS 300 instrument fitted with a 7.05 Tesla magnet from Oxford instruments, indirect detection probe and direct drive console including PFG module, or
an Agilent MercuryPlus 300 instrument fitted with a 7.05 Tesla magnet from Oxford instruments, 4 nuclei auto-switchable probe and Mercury plus console.

LC-MS

LC-MS Methods: Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃.H₂O in water (v/v); B: acetonitrile. Column: Kinetex EVO C18 2.1×30 mm, 5 μm.

Preparative Reversed Phase HPLC General Methods

Acidic prep HPLC (x-y% MeCN in water): Waters X-Select CSH column C18, 5 μm (19×50 mm), flow rate 28 mL min⁻¹ eluting with a H₂O-MeCN gradient containing 0.1% v/v formic acid over 6.5 min using UV detection at 254 nm. Gradient information: 0.0-0.2 min, x % MeCN; 0.2-5.5 min, ramped from x % MeCN to y % MeCN; 5.5-5.6 min, ramped from y % MeCN to 95% MeCN; 5.6-6.5 min, held at 95% MeCN.

Acidic prep HPLC (x-y % MeOH in water): Waters X-Select CSH column C18, 5 μm (19×50 mm), flow rate 28 mL min⁻¹ eluting with a 10 mM aq formic acid-gradient over 7.5 min using UV detection at 254 nm. Gradient information: 0.0-1.5 min, x % MeOH; 1.5-6.8 min, ramped from x % MeOH to y % MeOH; 6.8-6.9 min, ramped from y % MeOH to 95% MeOH; 6.9-7.5 min, held at 95% MeOH.

Basic prep HPLC (x-y % MeCN in water): Waters X-Bridge Prep column C18, 5 μm (19×50 mm), flow rate 28 mL min⁻¹ eluting with a 10 mM NH₄HCO₃-MeCN gradient over 6.5 min using UV detection at 254 nm. Gradient information: 0.0-0.2 minl x % MeCN; 0.2-5.5 min, ramped from x % MeCN to y % MeCN; 5.5-5.6 min, ramped from y % MeCN to 95% MeCN; 5.6-6.5 min, held at 95% MeCN.

### Synthesis of Intermediates

### Intermediate L1: (4-(Dimethylamino)pyridin-1-ium-1-carbonyl)(methylsulfonyl)amide

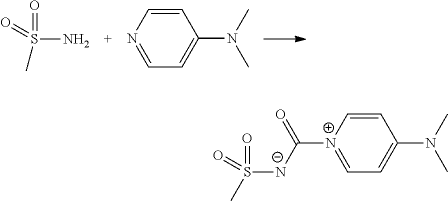

A solution of methanesulfonamide (1.7 g, 17.87 mmol) and DMAP (4.37 g, 35.7 mmol) in MeCN (25 mL) was stirred at room temperature for 10 minutes. Diphenyl carbonate (4.21 g, 19.66 mmol) was then added and the reaction was stirred at room temperature for 5 days. The precipitate was filtered off, washed with MTBE and dried in vacuo to afford the title compound (1.67 g, 38%) as a white solid.

1H NMR (CDCl₃) δ 9.07 (d, J=7.4 Hz, 2H), 6.74 (d, J=7.5 Hz, 2H), 3.35 (s, 6H), 3.20 (s, 3H).

The following intermediates were prepared according to the general procedure of Intermediate L1:

| Int. | Structure and name | Characterisation and procedure |
|---|---|---|
| L2 | 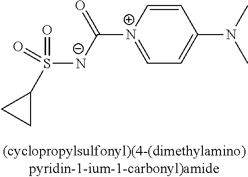 [SMILES: CN(C)c1cc[n+](C(=O)[N-]S(=O)(=O)C2CC2)cc1] | ¹H NMR (CDCl₃) δ 9.08 (d, J = 7.4 Hz, 2H), 6.72 (d, J = 7.5 Hz, 2H), 3.34 (s, 6H), 3.03 (tt, J = 8.1, 4.9 Hz, 1H), 1.36-1.29 (m, 2H), 0.99-0.90 (m, 2H). From cyclopropanesulfonamide |
| L3 | 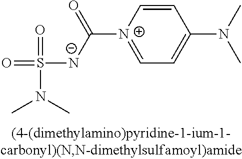 [SMILES: CN(C)c1cc[n+](C(=O)[N-]S(=O)(=O)N(C)C)cc1] | From (N-methyl-N- aminosulfoamino)methane |
| L4 | 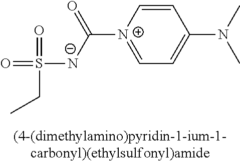 [SMILES: CCS(=O)(=O)[N-]C(=O)[n+]1ccc(N(C)C)cc1] | From ethanesulfonamide |
| L5 | 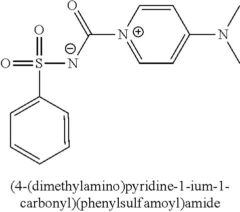 [SMILES: CN(C)c1cc[n+](C(=O)[N-]S(=O)(=O)c2ccccc2)cc1] | From benzenesulfonamide |
| L6 | 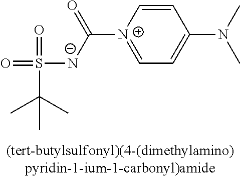 [SMILES: CN(C)c1cc[n+](C(=O)[N-]S(=O)(=O)C(C)(C)C)cc1] | ¹H NMR (CDCl₃) δ 8.24 (s, 2H), 6.55 (s, 2H), 3.06 (s, 6H), 1.48 (s, 9H). From 2-methyl-2-propanesulfonamide |

### Intermediate L7: 4-(2-Hydroxypropan-2-yl)furan-2-sulfonamide

### Step A: Ethyl furan-3-carboxylate

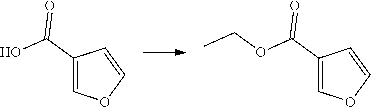

To a mixture of furan-3-carboxylic acid (50 g, 446.10 mmol, 1 eq) in EtOH (500 mL) was added dropwise H₂SO₄ (89.29 g, 892.20 mmol, 98% purity in solution, 2 eq) at 25° C. Then the reaction mixture was heated to 75° C. and stirred for 2.5 hours. The mixture was poured into ice water (200 mL) and extracted with EtOAc (_{3×200} mL). The organic phases were washed with 20% aqueous NaHCO₃ solution (2×200 mL), dried over anhydrous Na₂SO₄, filtered and concentrated in vacuo to give the title compound (50 g, 80%) as a yellow oil.

¹H NMR (400 MHz, CDCl₃) δ 8.01 (d, 1H), 7.43 (t, 1H), 6.75 (t, 1H), 4.3¹ (q, 2H) and 1.35 (S, 3H).

### Step B: 4-(Ethoxycarbonyl)furan-2-sulfonic acid

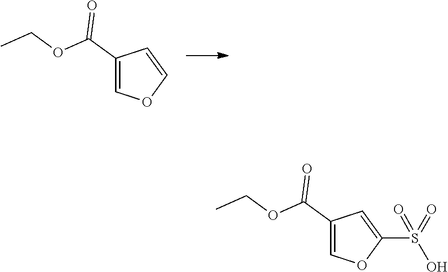

To a mixture of ethyl furan-3-carboxylate (45 g, 321.12 mmol, 1 eq) in DCM (500 mL) at −10° C. was added dropwise sulfurochloridic acid (46.77 g, 401.39 mmol, 1.25 eq) under N₂. After 15 minutes, the reaction mixture was stirred at 20° C. for 24 hours. Then the reaction mixture was filtered and the filter cake was dried in vacuo to give the title compound (55 g, 78%) as a white solid.

1H NMR (400 MHz, D₂O) δ 8.19 (s, 1H), 7.10 (s, 1H), 4.27 (q, 2H) and 1.27 (t, 3H).

### Step C: Ethyl 5-(chlorosulfonyl)furan-3-carboxylate

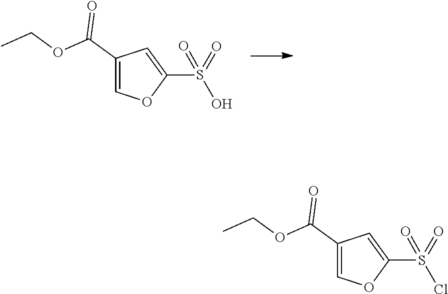

To a mixture of 4-(ethoxycarbonyl)furan-2-sulfonic acid (55 g, 249.77 mmol, 1 eq) in DCM (350 mL) at −10° C. was added dropwise pyridine (20.74 g, 262.26 mmol, 1.05 eq) under N₂. After 15 minutes, PCl₅ (54.61 g, 262.26 mmol, 1.05 eq) was added and the resulting mixture was stirred for another 15 minutes. Then the reaction mixture was warmed to 20° C. and stirred for 12 hours. The mixture was quenched with water (200 mL) and extracted with DCM (2×200 mL). Then the combined organic phases were washed with brine (100 mL), dried over anhydrous Na₂SO₄, filtered and concentrated in vacuo to give the title compound (35 g, 59%) as a yellow oil, which was used directly in the next step without further purification.

### Step D: Ethyl 5-sulfamoylfuran-3-carboxylate

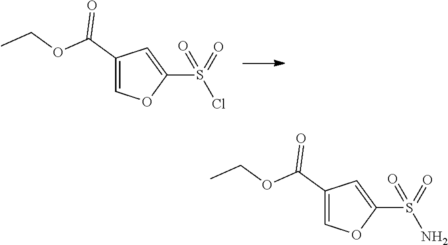

NH₃ (15 psi) was bubbled into a solution of ethyl 5-(chlorosulfonyl)furan-3-carboxylate (35 g, 146.66 mmol, 1 eq) in DCM (300 mL) at 0° C. for 15 minutes. Then the reaction mixture was stirred at 20° C. for 45 minutes. The mixture was filtered and the filtrate was concentrated in vacuo. The residue was purified by trituration with DCM (200 mL).The mixture was filtered and the filter cake was dried in vacuo to give the title compound (24 g, 75%) as a white solid.

1H NMR (400 MHz, DMSO-d₆) δ 8.63 (s, 1H), 7.93 (s, 2H), 7.12 (s, 1H), 4.27 (q, 2H) and 1.28 (t, 3H).

### Step E: 4-(2-Hydroxypropan-2-yl)furan-2-sulfonamide

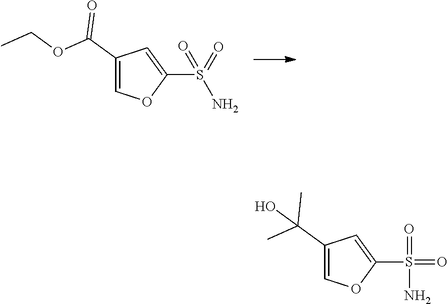

To a mixture of ethyl 5-sulfamoylfuran-3-carboxylate (24 g, 109.48 mmol, 1 eq) in THF (500 mL) was added dropwise over a period of 3o minutes MeMgBr (3 M, 164.22 mL, 4.5 eq) at −10° C. under N₂. The mixture was stirred at 0° C. for 30 minutes, then warmed to 20° C. and stirred for 12 hours. The mixture was poured slowly into ice-water (300 mL) and extracted with EtOAc (2×300 mL).The organic phases were washed with brine (loo mL), dried over anhydrous Na₂SO₄, filtered and concentrated in vacuo. The residue was purified by trituration with a mixture of n-hexane: EtOAc (v:v 20:1, 300 mL). The mixture was filtered and the filter cake was dried in vacuo to give the title compound (22 g, 97% yield, 99.3% purity on LCMS) as a white solid.

¹H NMR (400 MHz, DMSO-d₆) δ 7.68 (s, 1H), 7.65 (br s, 2H), 6.94 (s, 1H), and 1.38 (s, 6H).

### Intermediate L8: 1-Cyclopropyl-1H-pyrazole-3-sulfonamide

### Step A: 1-Cyclopropyl-3-nitro-1H-pyrazole

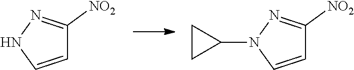 [SMILES: O=[N+]([O-])c1cc[nH]n1 ; O=[N+]([O-])c1ccn(C2CC2)n1]

To a solution of cyclopropylboronic acid (36.77 g, 428.04 mmol, 1.1 eq) in DCE (500 mL) was added 3-nitro-1H-pyrazole (44 g, 389.12 mmol, 1 eq), 2,2-bipyridine (60.77 g, 389.12 mmol, 1 eq) and Na₂CO₃ (64.59 g, 609.44 mmol, 1.57 eq) at 25° C. The mixture was stirred at 25° C. for 30 minutes. Then Cu(OAc)₂ (70.68 g, 389.12 mmol, 1 eq) was added, and the reaction mixture was heated to 70° C. and stirred for 15.5 hours. The reaction mixture was concentrated under reduced pressure. The residue was purified by column chromatography (SiO₂, petroleum ether: ethyl acetate, 30:1 to 3:1) to give impure product (26.7 g). The impure product was dissolved in pyrrolidine (10 mL), and the resulting mixture was stirred at 70° C. for 2 hours. The reaction mixture was concentrated under reduced pressure to remove pyrrolidine. The residue was diluted with H₂O (33 mL) and the pH was adjusted to 5-6 with 1M aqueous HCl solution. The mixture was extracted with EtOAc (_{3×50} mL). The combined organic layers were washed with brine (2×33 mL), dried over Na₂SO₄, filtered and concentrated under reduced pressure to give the title compound (17.7 g, 30%) as a yellow oil.

¹H NMR (400 MHz, CDCl₃) δ 7.54 (d, 1H), 6.84 (d, 1H), 3.73-3.67 (m, 1H), 1.24-1.22 (m, 2H) and 1.13-1.07 (m, 2H).

### Step B: 1-Cyclopropyl-1H-pyrazol-3-amine

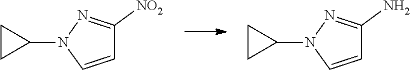 [SMILES: O=[N+]([O-])c1ccn(C2CC2)n1 ; Nc1ccn(C2CC2)n1]

To a solution of 1-cyclopropyl-3-nitro-1H-pyrazole (36 g, 235.08 mmol, 1 eq) in EtOH (400 mL) was added a solution of NH₄Cl (62.87 g, 1.18 mol, 5 eq) in H₂O (150 mL). Then the reaction mixture was heated to 60° C. and iron powder (39.38 g, 705.24 mmol, 3 eq) was added in portions. The reaction mixture was stirred at 60° C. for 16 hours. Then the reaction mixture was concentrated under reduced pressure. The residue was diluted with H₂O (500 mL), and the mixture was extracted with EtOAc (3×500 mL). The combined organic layers were washed with brine (2×250 mL), dried over Na₂SO₄, filtered and concentrated under reduced pressure. The residue was purified by column chromatography (SiO₂, petroleum ether:ethyl acetate, 30:1 to 1:1) to give the title compound (20 g, 69%) as a yellow oil.

¹H NMR (400 MHz, CDCl₃) δ 7.14 (d, 1H), 5.11 (d, 1H), 3.57 (br s, 2H), 3.38-3.32 (m, 1H), 0.99-0.95 (m, 2H) and 0.90-0.87 (m, 2H).

LCMS: m/z 124.2 (M+H)⁺(ES⁺).

### Step C: 1-Cyclopropyl-1H-pyrazole-3-sulfonyl chloride

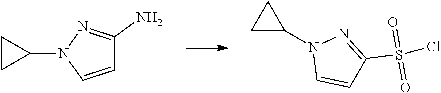 [SMILES: Nc1ccn(C2CC2)n1 ; O=S(=O)(Cl)c1ccn(C2CC2)n1]

To a solution of 1-cyclopropyl-1H-pyrazol-3-amine (19 g, 154.28 mmol, 1 eq) in MeCN (500 mL) and H₂O (50 mL) at 0° C. was added concentrated HCl solution (50 mL, 36 wt % aqueous solution). Then a solution of NaNO₂ (12.77 g, 185.13 mmol, 1.2 eq) in H₂O (50 mL) was added slowly. The resulting solution was stirred at 0° C. for 40 minutes. AcOH (50 mL), CuCl₂ (10.37 g, 77.14 mmol, 0.5 eq) and CuCl (763 mg, 7.71 mmol, 0.05 eq) were added. Then SO₂ gas (15 psi) was bubbled into the resulting mixture at 0° C. for 20 minutes. The resulting reaction mixture was stirred at 0° C. for 1 hour. The reaction mixture was concentrated under reduced pressure. The residue was diluted with H₂O (250 mL) and extracted with EtOAc (3×250 mL). The combined organic layers were washed with brine (2×150 mL), dried over Na₂SO₄, filtered and concentrated under reduced pressure. The residue was purified by column chromatography (SiO₂, petroleum ether: ethyl acetate, 1:0 to 1:1) to give the title compound (14 g, 44%) as a yellow oil.

¹H NMR (400 MHz, CDCl₃) δ 7.62 (d, 1H), 6.83 (d, 1H), 3.78-3.72 (m, 1H), 1.28-1.24 (m, 2H) and 1.16-1.12 (m, 2H).

### Step D: 1-Cyclopropyl-N,N-bis(4-methoxybenzyl)-1H-pyrazole-3-sulfonamide

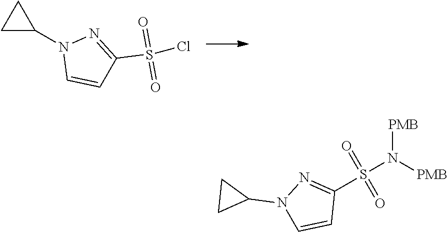

To a solution of 1-cyclopropyl-1H-pyrazole-3-sulfonyl chloride (28 g, 135.49 mmol, 1 eq) in THF (₃00 mL) was added TEA (27.42 g, 270.99 mmol, 2 eq) and bis(4-methoxybenzyl)amine (34.87 g, 135.49 mmol, 1 eq). The reaction mixture was stirred at 25° C. for 1 hour, diluted with H₂O (500 mL) and extracted with EtOAc (3×500 mL). The combined organic layers were washed with brine (2×500 mL), dried over Na₂SO₄, filtered and concentrated under reduced pressure. The residue was purified by reversed phase flash chromatography (0.5% NH₃.H₂O-MeCN) and the collected eluting solution was concentrated under reduced pressure to remove most of MeCN. Then the mixture was extracted with EtOAc (3×1 L). The combined organic layers were washed with brine (2×500 mL), dried over Na₂SO₄, filtered and concentrated under reduced pressure to give the title compound (30 g, 52% yield, 99.8% purity on HPLC).

¹H NMR (400 MHz, CDCl₃) δ 7.49 (d, 1H), 7.08-7.06 (m, 4H), 6.79-6.77 (m, 4H), 6.62 (d, 1H), 4.32 (s, 4H), 3.80 (s, 6H), 3.68-3.64 (m, 1H), 1.15-1.13 (m, 2H) and 1.09-1.06 (m, 2H).

LCMS: m/z 428.2 (M+H)⁺(ES⁺).

### Step E: 1-Cyclopropyl-1H-pyrazole-3-sulfonamide

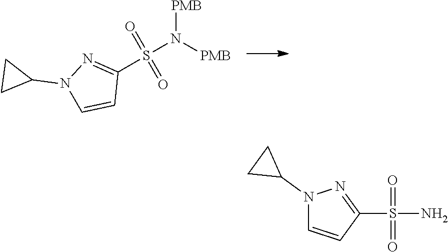

To a mixture of 1-cyclopropyl-N,N-bis(4-methoxybenzyl)-1H-pyrazole-3-sulfonamide g, 2.34 mmol, 1 eq) in DCM (10 mL) was added TFA (15.40 g, 135.06 mmol, 57.74 eq). The mixture was stirred at 25° C. for 12 hours. Most of the solvent was evaporated, and the residue was re-dissolved in MeOH (30 mL). Solids were formed and the reaction mixture was filtered. The filtrate was concentrated in vacuo, and the residue was triturated with a mixture of petroleum ether and EtOAc (30 mL, 20:1) to give the title compound (430 mg, 88% yield, 90% purity on LCMS) as a white solid.

¹H NMR (DMSO-d₆) δ 7.92 (s, 1H), 7.38 (br s, 2H), 6.55 (s, 1H), 3.84-3.78 (m, 1H) and 1.10-0.98 (m, 4H).

### Intermediate L9: 1-Methyl-3-[methyl(sulfamoyl)amino]pyrrolidine

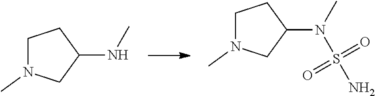

To a solution of N,1-dimethylpyrrolidin-3-amine (4 g, 35.03 mmol, 1 eq) in 1,2-dimethoxyethane (80 mL) was added sulfuric diamide (4.04 g, 42.04 mmol, 1.2 eq) in one portion. The reaction mixture was heated to 90° C. and stirred for 12 hours under N₂. Then the reaction mixture was concentrated in vacuo. The residue was purified by column chromatography (SiO₂, EtOAc: EtOH, 20:1 to 5:1) to give the title compound (3.5 g, 43% yield, 83% purity on LCMS) as a brown oil.

¹H NMR (400 MHz, DMSO-d₆) δ 6.65 (s, 2H), 4.31-4.23 (m, 1H), 2.62 (s, 3H), 2.61-2.56 (m, 2H), 2.41-2.36 (m, 1H), 2.20 (s, 3H), 2.18-2.12 (m, 1H), 2.05-1.98 (m, 1H) and 1.78-1.71 (m, 1H).

LCMS: m/z 194.0 (M+H)⁺(ES⁺).

### Intermediate L10: Benzenesulfinamide

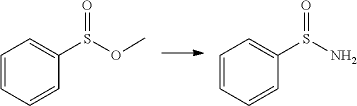

To a solution of methyl benzenesulfinate (500 mg, 3.20 mmol, 1 eq) in THF (10 mL) was added with LiHMDS (1M, 4.80 mL, 1.5 eq) at −78° C. The reaction mixture was stirred at −78° C. for 2 hours. Then a solution of NH₄Cl (342 mg, 6.40 mmol, 2 eq) in H₂O (5 mL) was added, and the resulting mixture was stirred at 25° C. for 1 hour. The reaction mixture was quenched with water (20 mL), and extracted with ethyl acetate (3×20 mL). The organic layers were dried over anhydrous Na₂SO₄, filtered and concentrated to give the title compound (400 mg, 89%) as a white solid.

¹H NMR (400 MHz, CDCl₃) δ 7.78-7.74 (m, 2H), 7.54-7.51 (m, 3H) and 4.36 (br s, 2H).

LCMS: m/z 141.9 (M+H)⁺(ES⁺).

### Intermediate L11: Methanesulfinamide

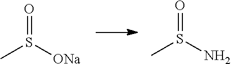

Ammonia gas (15 psi) was bubbled into THF (10 mL) at −78° C. for 10 minutes. Oxalyl chloride (39.18 mmol, 3.4 mL, 2 eq) was added into a solution of sodium methanesulfinate (2 g, 19.59 mmol, 1 eq) in THF (20 mL) at 0° C. under nitrogen. The mixture was stirred at 0° C. for 1 hour. Then the mixture was dropped into the above NH₃/THF solution at 0° C. The resulting mixture was stirred at 20° C. for 12 hours. A solid formed. The reaction mixture was filtered, and the filtrate was concentrated in vacuo to afford the title compound (0.9 g, crude) as a yellow solid.

¹H NMR (400 MHz, CDCl₃) δ 4.30 (br s, 2H) and 2.66 (s, 3H).

### Intermediate R1: 5-Bromo-6-methyl-2,3-dihydro-1H-inden-4-amine

### Step A: N-(6-Bromo-4-nitro-2,3-dihydro-1H-inden-5-yl)acetamide

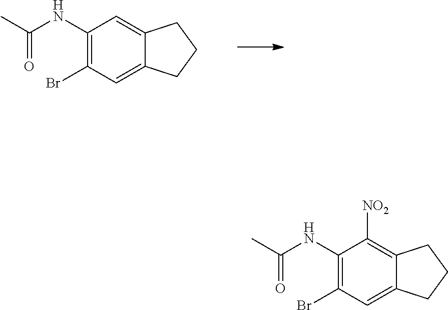 [SMILES: CC(=O)Nc1cc2c(cc1Br)CCC2 ; CC(=O)Nc1c(Br)cc2c(c1[N+](=O)[O-])CCC2]

Nitric acid (150 mL, 2350 mmol) was slowly added to sulfuric acid (150 mL) cooled to 0° C., while keeping the temperature below 20° C. The mixture was stirred for 10 minutes and added dropwise to a stirred mixture of N-(6-bromo-2,3-dihydro-1H-inden-5-yl)acetamide (58 g, 228 mmol) in AcOH (300 mL) and sulfuric acid (150 mL), keeping the temperature below 30° C. The reaction mixture was stirred at room temperature for 4 hours and then poured onto ice/water (4.5 L total volume, 2.5 kg ice) and left to stand at room temperature for 18 hours. The solid was filtered, washed with water (2.5 L), and dried to afford the title compound (55 g, 80%) as an ochre powder.

¹H NMR (DMSO-d6) δ 9.99 (s, 1H), 7.85 (s, 1H), 3.01-2.88 (m, 4H), 2.07 (p, J=7.5 Hz, 2H), 2.00 (s, 3H).

### LCMS m/z 299.0/301.0 (M+H)⁺(ES⁺).

### Step B: N-(6-Methyl-4-nitro-2,3-dihydro-1H-inden-5-yl)acetamide

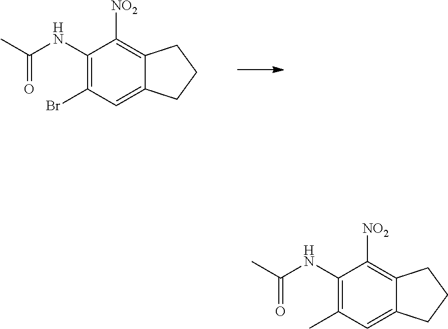 [SMILES: CC(=O)Nc1c(Br)cc2c(c1[N+](=O)[O-])CCC2 ; CC(=O)Nc1c(C)cc2c(c1[N+](=O)[O-])CCC2]

A mixture of N-(6-bromo-4-nitro-2,3-dihydro-1H-inden-5-yl)acetamide (30 g, 100 mmol), 2,4,6-trimethyl-1,3,5,2,4,6-trioxatriborinane (14.02 mL, 100 mmol) and K₂CO₃ (34.7 g, 251 mmol) in dioxane (500 mL) and H₂O (140 mL) was degassed with N₂ for 15 minutes. PdCl₂(dppf)-CH₂Cl₂ adduct (4.10 g, 5.01 mmol) was added. The reaction mixture was heated at 100° C. for 16 hours, diluted with brine (300 mL), and extracted with EtOAc (2×800 mL). The organic layers were dried (MgSO₄) and evaporated. The residue was triturated with EtOAc/isohexane (1:1 mixture, 400 mL) and the resultant solid was filtered, rinsing with hexanes, and dried in vacuo to afford the title compound (15.33 g, 56%) as a brown solid.

¹H NMR (DMSO-d6) δ 9.65 (s, 1H), 7.41 (s, 1H), 2.98-2.87 (m, 4H), 2.20 (s, 3H), 2.07-2.03 (m, 2H), 1.99 (s, 3H).

LCMS m/z 235.2 (M+H)⁺(ES⁺).

### Step C: 6-Methyl-4-nitro-2,3-dihydro-1H-inden-5-amine

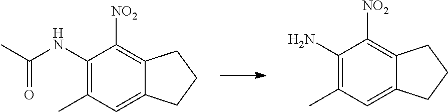 [SMILES: CC(=O)Nc1c(C)cc2c(c1[N+](=O)[O-])CCC2 ; Cc1cc2c(c([N+](=O)[O-])c1N)CCC2]

N-(6-Methyl-4-nitro-2,3-dihydro-1H-inden-5-yl)acetamide (15.33 g, 65.4 mmol) was suspended in a mixture of EtOH (126 mL) and concentrated aq HCl (126 mL). The mixture was heated to reflux overnight and concentrated in vacuo. The residue was basified by portionwise addition of 2M aq NaOH (500 mL). The aqueous layer was extracted with DCM (5×200 mL), dried (MgSO₄) and concentrated in vacuo to afford the title compound (15.18 g, 84%) as a brown solid.

¹H NMR (DMSO-d6) δ 7.21 (s, 1H), 6.61 (s, 2H), 3.16 (t, J=7.5 Hz, 2H), 2.76 (t, J=7.6 Hz, 2H), 2.16 (s, 3H), 2.00-1.94 (m, 2H).

LCMS m/z 193.4 (M+H)⁺(ES⁺).

### Step D: 5-Bromo-6-methyl-4-nitro-2,3-dihydro-1H-indene

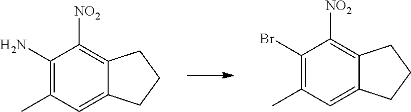

A solution of 6-methyl-4-nitro-2,3-dihydro-1H-inden-5-amine (4.9 g, 20.39 mmol) and isopentyl nitrite (3 mL, 22.33 mmol) in MeCN (400 mL) was heated to 55° C., whereupon CuBr₂ (4.56 g, 20.39 mmol) was added. The reaction mixture was heated to 70° C. and stirred for 1 hour. The reaction mixture was allowed to cool to room temperature and 1M HCl (200 mL) was added. The reaction mixture was extracted with DCM (3×200 mL). The organic phases were concentrated in vacuo and the crude product was purified by flash chromatography (0-20% EtOAc/isohexane) to afford the title compound (3.2 g, 60%) as a pale yellow solid.

¹H NMR (DMSO-d6) δ 7.50 (s, 1H), 2.94-2.86 (m, 4H), 2.41 (s, 3H), 2.09 (p, J=7.6 Hz, 2H).

LCMS m/z 279.2 (M+Na)⁺(ES⁺).

### Step E: 5-Bromo-6-methyl-2,3-dihydro-1H-inden-4-amine

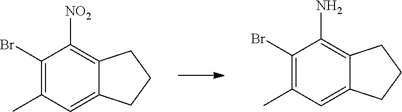

A stirred mixture of 5-bromo-6-methyl-4-nitro-2,3-dihydro-1H-indene (8.42 g, 32.9 mmol), saturated aq NH₄Cl (50 mL) and iron powder (7.34 g, 132 mmol) in EtOH/water (3:2, 80 mL) was stirred at 80° C. for 2 hours. After cooling to room temperature, the reaction was diluted with EtOAc (20 mL), and filtered through a pad of Celite®. The filtrate was diluted with water (10 mL). The layers were separated and the organic layer was dried (MgSO₄) and concentrated in vacuo. The residue was purified by flash chromatography (0-50% EtOAc/isohexane) to afford the title compound (6.52 g, 75%) as a pink solid.

¹H NMR (DMSO-d6) δ 6.48 (s, 1H), 4.94 (br s, 2H), 2.73 (t, J=7.5 Hz, 2H), 2.68 (t, J=7.4 Hz, 2H), 2.24 (s, 3H), 2.02-1.95 (m, 2H).

LCMS m/z 226/228 (M+H)⁺(ES⁺).

### Intermediate R2: 2-Bromo-5-cyclopropyl-4-fluoroaniline

### Step A: 3-Cyclopropyl-4-fluoroaniline

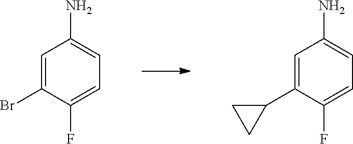 [SMILES: Nc1ccc(F)c(Br)c1 ; Nc1ccc(F)c(C2CC2)c1]

A mixture of 3-bromo-4-fluoroaniline (5 g, 26.3 mmol), cyclopropylboronic acid (2.7 g, 31.4 mmol) and K₂CO₃ (11 g, 80 mmol) in dioxane (100 mL) and water (20 ml) was degassed with N₂ for 10 minutes. PdCl₂(dppf) (0.96 g, 1.312 mmol) was added and the reaction mixture heated at 80° C. for 16 hours. Additional cyclopropylboronic acid (2.7 g, 26.3 mmol) and additional PdCl₂(dppf) (0.96 g, 26.3 mmol) were added and the reaction mixture heated at 80° C. for 48 hours. Then the reaction mixture was cooled to room temperature and partitioned between EtOAc (100 mL) and water (100 mL). The organic phase was washed with saturated brine (2×100 mL), dried (MgSO₄) and concentrated in vacuo. The crude product was purified by flash chromatography on silica gel (0-50% EtOAc/isohexane) to afford the title compound (1.87 g, 42%) as a brown solid.

¹H NMR (DMSO-d₆) δ 6.79-6.71 (m, 1H), 6.35-6.28 (m, 1H), 6.12 (dd, J=7.0, 2.7 Hz, 1H), 4.78 (s, 2H), 1.96-1.88 (m, 1H), 0.94-0.86 (m, 2H), 0.61- 0.55 (m, 2H).

LCMS m/z 152.1 (M+H)+(ES+).

### Step B: 2-Bromo-5-cyclopropyl-4-fluoroaniline

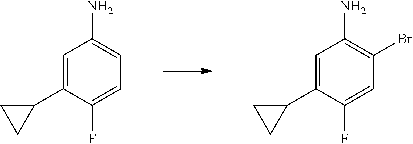 [SMILES: Nc1ccc(F)c(C2CC2)c1 ; Nc1cc(C2CC2)c(F)cc1Br]

3-Cyclopropyl-4-fluoroaniline (1.37 g, 8.07 mmol) and NBS (1.4 g, 7.87 mmol) in MeCN (20 mL) were stirred at room temperature for 16 hours. Then the reaction mixture was concentrated in vacuo and the crude product was purified by flash chromatography on silica gel (0-40% EtOAc/hexanes) to afford the title compound (1.04 g, 52%) as a pale tan solid.

¹H NMR (DMSO-d₆) δ 7.18 (d, J=9.7 Hz, 1H), 6.40 (d, J=7.4 Hz, 1H), 5.00 (s, 2H), 1.91 (tt, J=8.5, 5.2 Hz, 1H), 0.97-0.90 (m, 2H), 0.63-0.58 (m, 2H).

LCMS m/z 229.9/231.9 (M+H)+(ES+).

### Intermediate R3: 2-Bromo-4-fluoro-5-(trifluoromethoxy)aniline

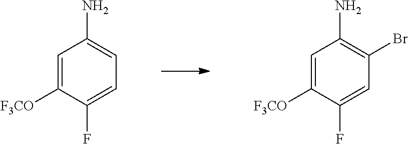 [SMILES: Nc1ccc(F)c(OC(F)(F)F)c1 ; Nc1cc(OC(F)(F)F)c(F)cc1Br]

4-Fluoro-3-(trifluoromethoxy)aniline (1 g, 5.13 mmol) and NBS (1 g, 5.62 mmol) in MeCN (50 mL) were stirred at room temperature for 3 hours. Volatiles were evaporated. The crude product was diluted with DCM (50 mL), washed with water (100 mL) and saturated aq Na₂S₂O₃ (100 mL), dried (MgSO₄) and concentrated in vacuo. The crude product was purified by flash chromatography on silica gel (0-50% EtOAc/isohexane) to afford the title compound (1.25 g, 88%) as a brown oil.

¹H NMR (DMSO-d₆) δ 7.62 (d, J=9.9 Hz, 1H), 6.94-6.87 (m, 1H), 5.53 (s, 2H).

LCMS m/z 273/275 (M+H)⁺(ES⁺).

### Intermediate R4: 2-Bromo-5-ethyl-4-fluoroaniline

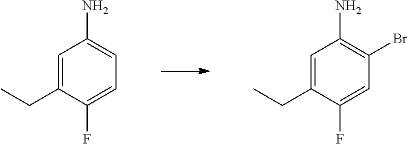

3-Ethyl-4-fluoroaniline (1.06 g, 7.62 mmol) and NBS (1.4 g, 7.87 mmol) in MeCN (25 mL) were stirred at room temperature for 3 hours. Volatiles were evaporated. The crude product was diluted with DCM (50 mL), washed with water (loo mL) and saturated aq Na₂S₂O₃ (100 mL), dried (MgSO₄) and concentrated in vacuo. The crude product was purified by flash chromatography on silica gel (0-40% EtOAc/isohexane), followed by another flash chromatography on silica gel (0-50% EtOAc/isohexane) to afford the title compound (1.39 g, 75%) as a brown oil.

¹H NMR (DMSO-d₆) δ 7.19 (d, J=9.4 Hz, 1H), 6.70 (d, J=7.3 Hz, 1H), 5.12 (s, 2H), 2.47 (q, J=7.4 Hz, 2H), 1.12 (t, J=7.5 Hz, 3H).

LCMS m/z 218/220 (M+H)⁺(ES⁺).

### Intermediate R5: 4-Bromo-3-methyl-2-((1-methylpiperidin-4-yl)oxy)pyridine

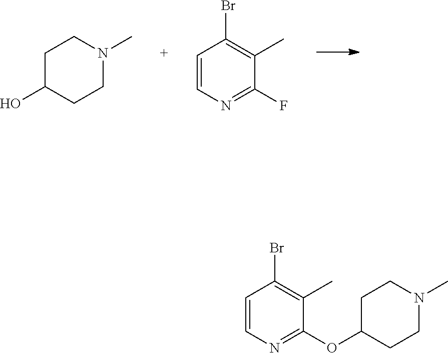

### 1-Methylpiperidin-4-ol (0.67 g, 5.79 mmol) was added to a mixture of KO^{t}Bu (0.89 g,

7.89 mmol) in THF (5 mL) at room temperature. The reaction mixture was stirred for 1 hour, and then cooled in an ice bath. A solution of 4-bromo-2-fluoro-3-methylpyridine (1 g, 5.26 mmol) in THF (5 mL) was added. The mixture was warmed to room temperature, stirred for 2 days, and then partitioned between EtOAc (20 mL) and water (20 mL). The aqueous phase was extracted with EtOAc (20 mL). The organic phases were combined, dried (MgSO₄) and concentrated in vacuo. The crude product was purified by flash chromatography (0-10% (0.7 M ammonia/)/DCM) to afford the title compound (1.30 g, 86%) as a colourless oil.

¹H NMR (DMSO-d6) δ 7.85 (dd, J=5.4, 0.8 Hz, 1H), 7.19 (d, J=5.4 Hz, 1H), 5.02 (tt, J=8.1, 4.0 Hz, 1H), 2.59-2.52 (m, 2H), 2.26-2.20 (m, 5H), 2.17 (s, 3H), 1.97-1.86 (m, 2H), 1.74-1.62 (m, 2H).

LCMS m/z 285.1/287.1 (M+H)⁺(ES⁺).

The following intermediate was prepared according to the general procedure of Intermediate R5:

| Int. | Structure and name | Characterisation and procedure |
|---|---|---|
| R6 | 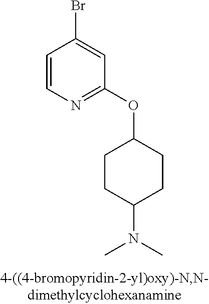 | ¹H NMR (DMSO-d6) δ 8.05 (d, J = 5.5 Hz, 1H), 7.18 (dd, J = 5.5, 1.7 Hz, 1H), 7.04 (d, J = 1.6 Hz, 1H), 4.92-4.85 (m, 1H), 2.23- 2.12 (m, 7H), 2.11-2.01 (m, 2H), 1.88-1.75 (m, 2H), 1.46-1.24 (m, 4H). LCMS m/z 299.1/301.1 (M + H)⁺ (ES⁺). |

### Intermediate R7: 5-Bromo-6-cyclopropyl-2,3-dihydro-1H-inden-4-amine

### Step A: N-(6-Cyclopropyl-4-nitro-2,3-dihydro-1H-inden-5-yl)acetamide

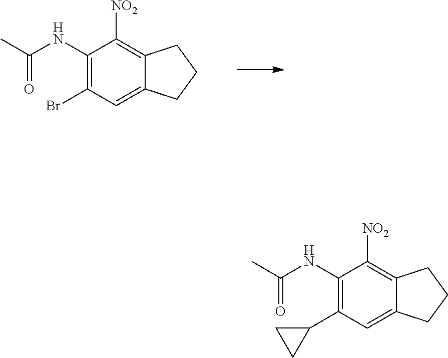 [SMILES: CC(=O)Nc1c(Br)cc2c(c1[N+](=O)[O-])CCC2 ; CC(=O)Nc1c(C2CC2)cc2c(c1[N+](=O)[O-])CCC2]

N₂ was bubbled through a stirred mixture of N-(6-bromo-4-nitro-2,3-dihydro-1H-inden-5-yl)acetamide (Intermediate R1, step A) (1 g, 3.34 mmol), cyclopropylboronic acid (0.35 g, 4.01 mmol) and K₂CO₃ (1.39 g, 10.03 mmol) in dioxane (35 ml) and water (10 ml) for 10 minutes. PdCl₂(dppf) (0.122 g, 0.167 mmol) was added. Then the reaction mixture was heated at 80° C. for 4 hours, cooled to room temperature, and partitioned between EtOAc (100 mL) and water (100 mL). The organic layer was dried (MgSO₄) and evaporated. The crude product was purified by flash chromatography on silica gel (0-100% EtOAc/isohexane) to afford the title compound (120 mg, 13%) as a yellow solid.

¹H NMR (DMSO-d6) δ 9.76 (s, 1H), 7.09 (s, 1H), 2.98-2.88 (m, 4H), 2.09-1.94 (m, 6H), 1.00-0.89 (m, 2H), 0.68-0.60 (m, 2H).

LCMS m/z 261.2 (M+H)⁺(ES⁺).

### Step B: 6-Cyclopropyl-4-nitro-2,3-dihydro-1H-inden-5-amine

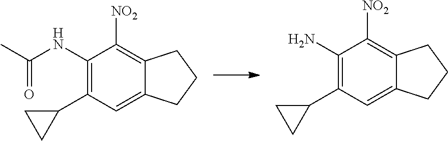 [SMILES: CC(=O)Nc1c(C2CC2)cc2c(c1[N+](=O)[O-])CCC2 ; Nc1c(C2CC2)cc2c(c1[N+](=O)[O-])CCC2]

N-(6-Cyclopropyl-4-nitro-2,3-dihydro-1H-inden-5-yl)acetamide (120 mg, 0.461 mmol) was suspended in H₂O (2 mL). Concentrated HCl (2 mL) was added slowly, whilst the reaction mixture was cooled in an ice bath. Then reaction mixture was stirred at 110° C. for 16 hours and cooled to 0° C. on ice. The reaction mixture was basified by portionwise addition of 50 wt % aqueous NaOH (˜50 mL by 10 mL increments). The aqueous mixture was extracted with DCM (5×200 mL). The combined organic layers were dried (MgSO₄) and concentrated in vacuo to afford the title compound (107 mg, 51%) as a brown solid.

¹H NMR (DMSO-d6) δ 7.14 (s, 1H), 6.76 (s, 2H), 3.16 (t, J=7.3 Hz, 2H), 2.76 (t, J=7.6 Hz, 2H), 1.97 (p, J=7.4 Hz, 2H), 1.76-1.65 (m, 1H), 0.96-0.90 (m, 2H), 0.59-0.47 (m, 2H).

LCMS m/z 219.4 M+H)⁺(ES⁺).

### Step C: 5-Bromo-6-cyclopropyl-4-nitro-2,3-dihydro-1H-indene

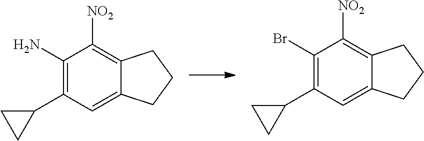 [SMILES: Nc1c(C2CC2)cc2c(c1[N+](=O)[O-])CCC2 ; O=[N+]([O-])c1c(Br)c(C2CC2)cc2c1CCC2]

A solution of 6-cyclopropyl-4-nitro-2,3-dihydro-1H-inden-5-amine (106 mg, 0.487 mmol) and isopentyl nitrite (72 μL, 0.536 mmol) in MeCN (7 mL) was heated to 55° C. Then CuBr₂ (109 mg, 0.487 mmol) was added, and the reaction mixture was heated to 70° C. and stirred for 1 hour. Then the reaction mixture was allowed to cool to room temperature. 1M HCl (10 mL) was added and the reaction mixture was extracted with DCM (3×20 mL). The combined organic phases were concentrated in vacuo to afford the title compound which was used crude in the next step.

### Step D: 5-Bromo-6-cyclopropyl-2,3-dihydro-1H-inden-4-amine

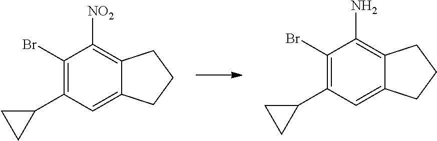 [SMILES: O=[N+]([O-])c1c(Br)c(C2CC2)cc2c1CCC2 ; Nc1c(Br)c(C2CC2)cc2c1CCC2]

A stirred mixture of 5-bromo-6-cyclopropyl-4-nitro-2,3-dihydro-1H-indene (104 mg, 0.369 mmol), saturated aqueous ammonium chloride (0.5 mL) and iron powder (82 mg, 1.474 mmol) in EtOH:water (3:2, 1 mL) was stirred at 80° C. for 2 hours. Then the reaction mixture was cooled to room temperature, diluted with EtOAc (20 mL) and filtered through a pad of Celite®. The filtrate was diluted with water (10 mL) and the organic layer was collected, dried (MgSO₄) and concentrated in vacuo. The crude product was purified by flash chromatography on silica gel (0-50% EtOAc/isohexane) to afford the title compound (17 mg, 11%) as a pink solid.

LCMS m/z 252/254 (M+H)⁺(ES⁺).

### Intermediate R8: 6-Methyl-5-(pyridin-4-yl)-2,3-dihydro-1H-inden-4-amine

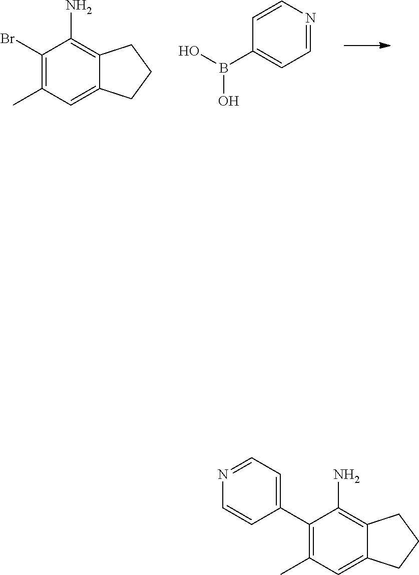 [SMILES: Cc1cc2c(c(N)c1Br)CCC2 ; OB(O)c1ccncc1 ; Cc1cc2c(c(N)c1-c1ccncc1)CCC2]

N₂ was bubbled through a stirred mixture of 5-bromo-6-methyl-2,3-dihydro-1H-inden-4-amine (Intermediate R1) (200 mg, 0.885 mmol), pyridin-4-ylboronic acid (120 mg, 0.973 mmol) and K₂CO₃ (367 mg, 2.65 mmol) in dioxane (30 mL) and water (5 mL) for 5 minutes. PdCl₂(dppf) (32.4 mg, 0.044 mmol) was added, and the reaction mixture was heated at 80° C. for 20 hours. Then the reaction mixture was cooled to room temperature, and partitioned between EtOAc (100 mL) and water (50 mL). The organic layer was dried (MgSO₄), evaporated and the residue was purified by flash chromatography on silica gel (0-40% EtOAc/isohexane) to afford the title compound (40 mg, 20%) as a yellow oil.

¹H NMR (DMSO-d6) δ 8.67-8.62 (m, 2H), 7.21-7.17 (m, 2H), 6.47 (s, 1H), 4.14 (s, 2H), 2.79 (t, J=7.5 Hz, 2H), 2.65 (t, J=7.3 Hz, 2H), 2.00 (p, J=7.4 Hz, 2H), 1.87 (s, 3H).

LCMS m/z 225.1 (M+H)⁺(ES⁺).

The following intermediates were prepared according to the general procedure of Intermediate R8:

| Int. | Structure and name | Characterisation and procedure |
|---|---|---|
| R9 | 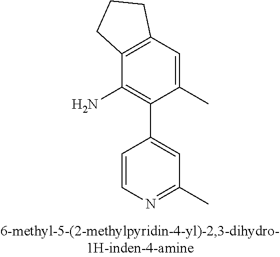 | ¹H NMR (DMSO-d6) δ 8.51 (d, J = 5.1 Hz, 1H), 7.05 (s, 1H), 6.98 (dd, J = 5.0, 1.6 Hz, 1H), 6.46 (s, 1H), 4.11 (s, 2H), 3.33 (s, 3H), 2.79 (t, J = 7.5 Hz, 2H), 2.65 (t, J = 7.4 Hz, 2H), 2.00 (p, J = 7.4 Hz, 2H), 1.87 (s, 3H). LCMS m/z 239.2 (M + H)⁺ (ES⁺). From Intermediate R₁ |
| R10 | 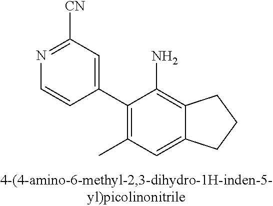 | ¹H NMR (DMSO-d6) δ 8.80-8.77 (m, 1H), 7.85 (s, 1H), 7.54 (dd, J = 5.0, 1.7 Hz, 1H), 6.47 (s, 1H), 4.40 (s, 2H), 2.79 (t, J = 7.5 Hz, 2H), 2.65 (t, J = 7.3 Hz, 2H), 2.00 (p, J = 7.4 Hz, 2H), 1.86 (s, 3H). LCMS m/z 250.4 (M + H)⁺ (ES⁺). From Intermediate R₁ |
| R11 | 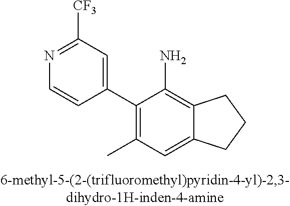 | ¹H NMR (DMSO-d6) δ 8.82 (d, J = 4.9 Hz, 1H), 7.65 (s, 1H), 7.53 (dd, J = 4.9, 1.5 Hz, 1H), 6.48 (s, 1H), 4.36 (s, 2H), 2.80 (t, J = 7.5 Hz, 2H), 2.66 (t, J = 7.3 Hz, 2H), 2.04-1.96 (m, 2H), 1.86 (s, 3H). LCMS m/z 293.5 (M + H)⁺ (ES⁺). From Intermediate R₁ |
| R12 | 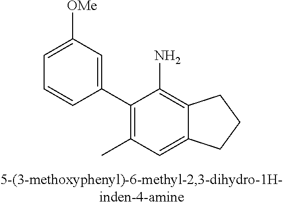 | ¹H NMR (DMSO-d6) δ 7.42-7.36 (m, 1H), 6.93 (ddd, J = 8.3, 2.7, 1.0 Hz, 1H), 6.71 (dt, J = 7.5, 1.3 Hz, 1H), 6.68 (dd, J = 2.6, 1.4 Hz, 1H), 6.45 (s, 1H), 3.96 (s, 2H), 3.77 (s, 3H), 2.79 (t, J = 7.5 Hz, 2H), 2.64 (t, J = 7.3 Hz, 2H), 2.00 (p, J = 7.4 Hz, 2H), 1.88 (s, 3H). LCMS m/z 254.2 (M + H)⁺ (ES⁺). From Intermediate R₁ |

### Intermediate R13: 5-(2-Cyclopropoxypyridin-4-yl)-6-methyl-2,3-dihydro-1H-inden-4-amine

### Step A: 4-Bromo-2-cyclopropoxypyridine

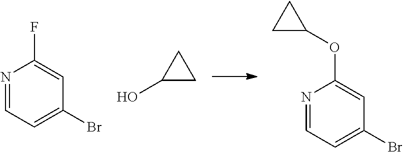

To a mixture of cyclopropanol (1 g, 17.22 mmol) and 4-bromo-2-fluoropyridine (1.2 ml, 11.68 mmol) in NMP (13 mL) was added potassium tert-butoxide (1.9 g, 16.93 mmol) portionwise. The resultant mixture was stirred at room temperature for 30 minutes under nitrogen. Then the reaction mixture was diluted with EtOAc (50 mL), washed with water (30 mL) and brine (30 mL), dried over anhydrous sodium sulfate, filtered and evaporated to afford the title compound (2.27 g, 83%) as a brown oil.

¹H NMR (DMSO-d6) δ 8.12 (d, J=5.4 Hz, 1H), 7.28 (dd, J=5.4, 1.7 Hz, 1H), 7.16 (d, J=1.6 Hz, 1H), 4.21 (tt, J=6.2, 3.0 Hz, 1H), 0.80-0.74 (m, 2H), 0.70-0.66 (m, 2H).

LCMS m/z 214/216 (M+H)⁺(ES⁺).

### Step B: 5-(2-Cyclopropoxypyridin-4-yl)-6-methyl-2,3-dihydro-1H-inden-4-amine

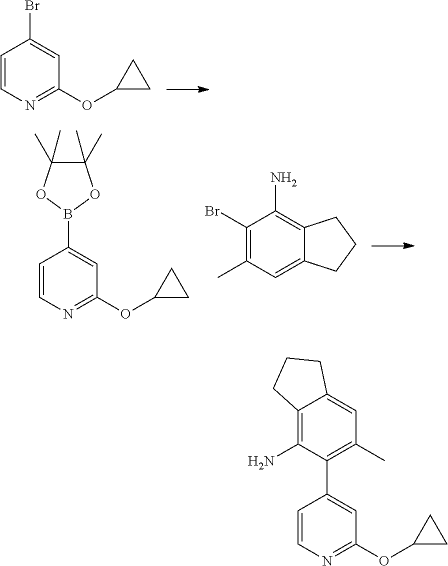

To a solution of 4-bromo-2-cyclopropoxypyridine (189 mg, 0.885 mmol) in dioxane (5 mL) was added B₂Pin₂ (247 mg, 0.973 mmol), followed by potassium acetate (347 mg, 3.54 mmol) and PdCl₂(dppf)-CH₂Cl₂ adduct (36 mg, 0.044 mmol). The reaction was degassed (N₂, 5 minutes), evacuated and backfilled with N₂ (×3) and stirred at 90° C. for 2 hours. Then the reaction mixture was cooled to room temperature. A solution of 5-bromo-6-methyl-2,3-dihydro-1H-inden-4-amine (Intermediate R1) (200 mg, 0.885 mmol) in dioxane (3 mL) was added, followed by a solution of potassium carbonate (367 mg, 2.65 mmol) in water (1.5 mL). The reaction mixture was stirred at 90° C. for 16 hours, diluted with brine (io mL), and extracted with DCM (2×20 mL). The organic layer was dried (MgSO₄), filtered and evaporated. The crude product was purified by flash chromatography (0-60% EtOAc/isohexane) to afford the title compound (135 mg, 52%) as a yellow oil.

¹H NMR (DMSO-d6) δ 8.26 (d, J=5.1 Hz, 1H), 6.81 (dd, J=5.1, 1.3 Hz, 1H), 6.63 (d, J=1.2 Hz, 1H), 6.45 (s, 1H), 4.22 (tt, J=6.3, 3.1 Hz, 1H), 4.16 (s, 2H), 2.78 (t, J=7.5 Hz, 2H), 2.64 (t, J=7.3 Hz, 2H), 2.02-1.95 (m, 2H), 1.88 (s, 3H), 0.81- 0.68 (m, 4H).

LCMS m/z 281.2 (M+H)+(ES+).

The following intermediates were prepared according to the general procedure of Intermediate R13:

| Int. | Structure and name | Characterisation and procedure |
|---|---|---|
| R14 | 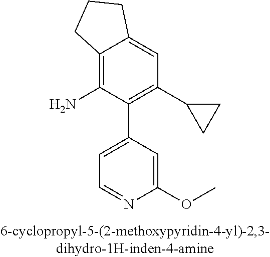 | LCMS m/z 281.2 (M + H)⁺ (ES⁺). From Intermediate R7 |
| R15 | 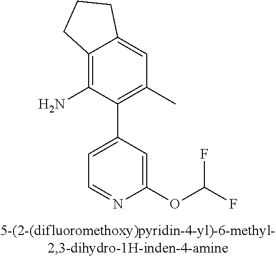 | ¹H NMR (DMSO-d6) δ 8.32 (d, J = 5.1 Hz, 1H), 7.76 (t, J = 72.9 Hz, 1H), 7.07 (d, J = 5.2 Hz, 1H), 6.87 (s, 1H), 6.46 (s, 1H), 4.27 (s, 2H), 2.79 (t, J = 7.5 Hz, 2H), 2.65 (t, J = 7.3 Hz, 2H), 2.04-1.96 (m, 2H), 1.89 (s, 3H). LCMS m/z 291.1 (M + H)⁺ (ES⁺). From Intermediate R1 |
| R16 | 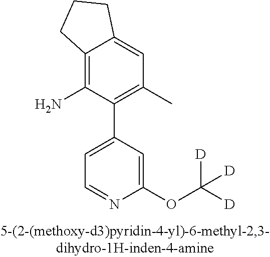 | ¹H NMR (DMSO-d6) δ 8.24 (d, J = 5.1 Hz, 1H), 6.77 (dd, J = 5.2, 1.2 Hz, 1H), 6.58 (t, J = 1.1 Hz, 1H), 6.45 (s, 1H), 4.16 (s, 2H), 2.78 (t, J = 7.5 Hz, 2H), 2.64 (t, J = 7.3 Hz, 2H), 2.01-1.93 (m, 2H), 1.88 (s, 3H). LCMS m/z 258.3 (M + H)⁺ (ES⁺). From Intermediate R1 |
| R17 | 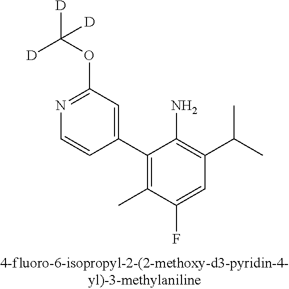 | ¹H NMR (DMSO-d6) δ 8.28 (d, J = 5.1 Hz, 1H), 6.88 (d, J = 11.3 Hz, 1H), 6.81 (d, J = 5.2 Hz, 1H), 6.65 (s, 1H), 4.01 (s, 2H), 3.04-2.95 (m, 1H), 1.79 (d, J = 2.1 Hz, 3H), 1.15 (d, J = 6.7 Hz, 6H). LCMS m/z 278.1 (M + H)⁺ (ES⁺). From Intermediate R33, step C |
| R18 | 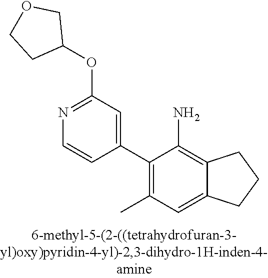 | ¹H NMR (DMSO-d6) δ 8.22 (d, J = 5.2 Hz, 1H), 6.77 (dd, J = 5.2, 1.4 Hz, 1H), 6.56 (s, 1H), 6.45 (s, 1H), 5.57-5.52 (m, 1H), 4.16 (s, 2H), 3.96-3.92 (m, 1H), 3.89-3.80 (m, 2H), 3.80-3.74 (m, 1H), 2.78 (t, J = 7.5 Hz, 2H), 2.64 (t, J = 7.3 Hz, 2H), 2.30- 2.21 (m, 1H), 2.09-1.96 (m, 3H), 1.88 (s, 3H). LCMS m/z 311.2 (M + H)⁺ (ES⁺). From Intermediate R1 |
| R19 | 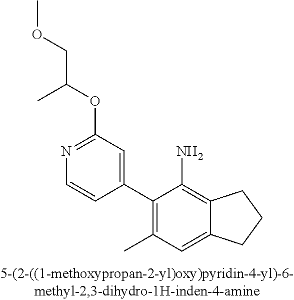 | ¹H NMR (DMSO-d6) δ 8.21 (d, J = 5.1 Hz, 1H), 6.74 (d, J = 5.2 Hz, 1H), 6.51 (s, 1H), 6.45 (s, 1H), 5.35 (s, 1H), 4.14 (s, 2H), 3.59- 3.53 (m, 1H), 3.50-3.45 (m, 1H), 3.30 (s, 3H), 2.78 (t, J = 7.4 Hz, 2H), 2.64 (t, J = 7.3 Hz, 2H), 2.04-1.93 (m, 2H), 1.88 (s, 3H), 1.30-1.26 (m, 3H). LCMS m/z 313.2 (M + H)⁺ (ES⁺). From Intermediate R1 |
| R20 | 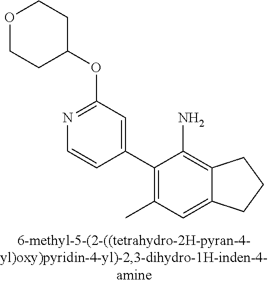 | ¹H NMR (DMSO-d6) δ 8.21 (d, J = 5.2 Hz, 1H), 6.75 (dd, J = 5.2, 1.4 Hz, 1H), 6.54 (s, 1H), 6.45 (s, 1H), 5.27-5.15 (m, 1H), 4.14 (s, 2H), 3.93-3.80 (m, 4H), 2.78 (t, J = 7.5 Hz, 2H), 2.65 (t, J = 7.3 Hz, 2H), 2.11-1.93 (m, 4H), 1.88 (s, 3H), 1.72-1.56 (m, 2H). LCMS m/z 325.2 (M + H)⁺ (ES⁺). From Intermediate R1 |
| R21 | 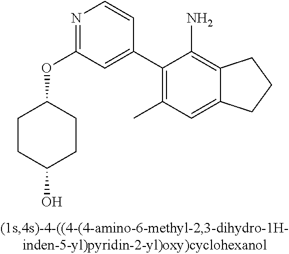 | ¹H NMR (DMSO-d6) δ 8.20 (d, J = 5.2 Hz, 1H), 6.72 (dd, J = 5.1, 1.4 Hz, 1H), 6.51 (s, 1H), 6.45 (s, 1H), 5.11-5.04 (m, 1H), 4.47 (d, J = 3.9 Hz, 1H), 4.14 (s, 2H), 3.67- 3.62 (m, 1H), 2.78 (t, J = 7.5 Hz, 2H), 2.68- 2.61 (m, 2H), 2.00 (q, J = 7.6 Hz, 2H), 1.94-1.85 (m, 5H), 1.73-1.66 (m, 2H), 1.64-1.58 (m, 4H). LCMS m/z 339.1 (M + H)⁺ (ES⁺). From Intermediate R1 |
| R22 | 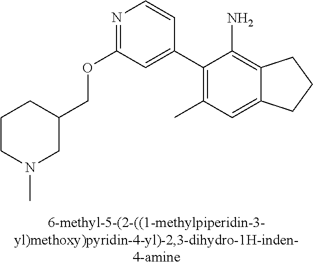 | ¹H NMR (DMSO-d6) δ 8.21 (d, J = 5.1 Hz, 1H), 6.75 (dd, J = 5.1, 1.4 Hz, 1H), 6.56 (s, 1H), 6.45 (s, 1H), 2.89-2.81 (m, 1H), 2.78 (t, J = 7.5 Hz, 2H), 2.64 (t, J = 7.4 Hz, 2H), 2.18 (s, 3H), 2.08-1.95 (m, 4H), 1.96- 1.90 (m, 1H), 1.88 (s, 3H), 1.86-1.77 (m, 2H), 1.77-1.69 (m, 2H), 1.69-1.60 (m, 2H), 1.56-1.44 (m, 2H). LCMS m/z 352.6 (M + H)⁺ (ES⁺). From Intermediate R1 |
| R23 | 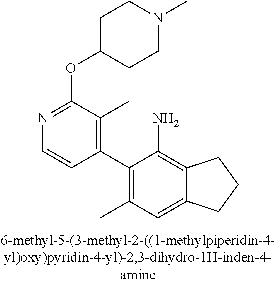 | LCMS m/z 352.2 (M + H)⁺ (ES⁺). From Intermediate R1 + Intermediate R5 |
| R24 | 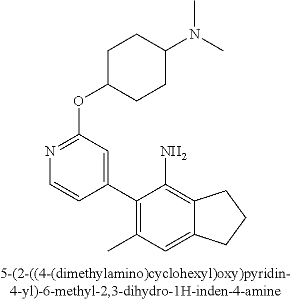 | ¹H NMR (DMSO-d₆) δ 8.19 (d, J = 5.1 Hz, 1H), 6.71 (dd, J = 5.1, 1.4 Hz, 1H), 6.48 (s, 1H), 6.44 (s, 1H), 4.98-4.87 (m, 1H), 4.12 (s, 2H), 2.78 (t, J = 7.6 Hz, 2H), 2.63 (t, J = 7.4 Hz, 2H), 2.25-2.05 (m, 9H), 1.98 (p, J = 7.5 Hz, 2H), 1.90-1.77 (m, 5H), 1.49- 1.26 (m, 4H). LCMS m/z 366.6 (M + H)⁺ (ES⁺). From Intermediate R1 + Intermediate R6 |

### Intermediate R25: 5-(2-Methoxypyridin-4-yl)-6-methyl-2,3-dihydro-1H-inden-4-amine

### Step A: 2-Methoxy-4-(6-methyl-4-nitro-2,3-dihydro-1H-inden-5-yl)pyridine

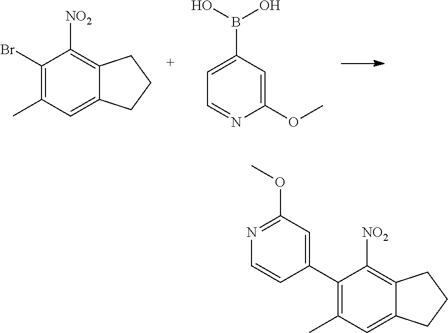

A mixture of 5-bromo-6-methyl-4-nitro-2,3-dihydro-1H-indene (Intermediate R1, Step D) (218 mg, 0.851 mmol), (2-methoxypyridin-4-yl)boronic acid (156 mg, 1.021 mmol) in dioxane (2.5 ml) and K₂CO₃ (353 mg, 2.55 mmol) in water (0.5 mL) was degassed with N₂ for 15 minutes. Then Pd(dppf)Cl₂. DCM (35 mg, 0.043 mmol) was added. The reaction mixture was heated to 80° C. for 2 hours, cooled to room temperature and partitioned between EtOAc (10 mL) and water (5 mL). The organic layer was washed with water (10 mL) and brine (10 mL), dried (MgSO₄) and evaporated to afford the title compound (186 mg, 63%) which was used in the next step without purification.

¹H NMR (DMSO-d6) δ 8.24 (d, J=5.2 Hz, 1H), 7.50 (s, 1H), 6.88-6.81 (m, 1H), 6.67 (d, J=2.0 Hz, 1H), 3.89 (s, 3H), 3.03-2.92 (m, 4H), 2.18-2.03 (m, 5H).

LCMS m/z 285.0 (M+H)⁺(ES⁺).

### Step B: 5-(2-Methoxypyridin-4-yl)-6-methyl-2,3-dihydro-1H-inden-4-amine

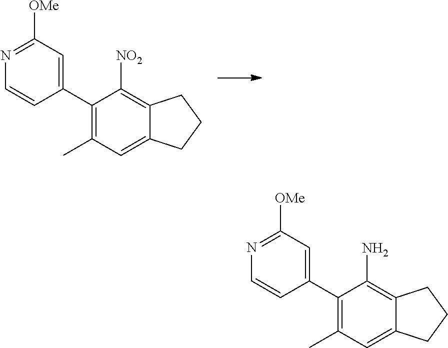 [SMILES: COc1cc(-c2c(C)cc3c(c2[N+](=O)[O-])CCC3)ccn1 ; COc1cc(-c2c(C)cc3c(c2N)CCC3)ccn1]

A mixture of 2-methoxy-4-(6-methyl-4-nitro-2,3-dihydro-1H-inden-5-yl)pyridine (186 mg, 0.536 mmol) and 5% Pd/C (Type 87L, 58.5% moisture) (55 mg, 10.72 mol) in EtOH (2 mL) was hydrogenated at 1 bar for 6 hours. Then the reaction mixture was filtered through Celite® and evaporated to afford the title compound (120 mg, 77%) which was used without purification.

¹H NMR (DMSO-d6) δ 8.24 (d, J=5.2 Hz, 1H), 6.77 (dd, J=5.2, 1.5 Hz, 1H), 6.58 (s, 1H), 6.45 (s, 1H), 4.16 (s, 2H), 3.89 (s, 3H), 2.78 (t, J=7.5 Hz, 2H), 2.64 (t, J=7.4 Hz, 2H), 1.99 (p, J=7.4 Hz, 2H), 1.88 (s, 3H).

LCMS m/z 255.1 (M+H)⁺(ES⁺).

The following intermediate was prepared according to the general procedure of Intermediate R25:

| Int. | Structure and name | Characterisation and procedure |
|---|---|---|
| R26 | 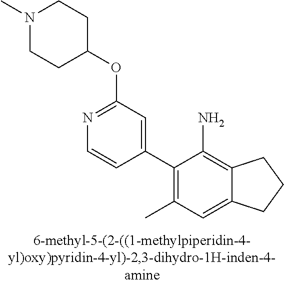 | ¹H NMR (DMSO-d6) δ 8.21 (d, J = 5.1 Hz, 1H), 6.73 (dd, J = 5.2, 1.4 Hz, 1H), 6.55- 6.47 (m, 1H), 6.45 (s, 1H), 5.01 (tt, J = 8.8, 4.2 Hz, 1H), 4.14 (s, 2H), 2.78 (t, J = 7.5 Hz, 2H), 2.74-2.58 (m, 4H), 2.27-2.09 (m, 5H), 2.06-1.93 (m, 4H), 1.88 (s, 3H), 1.76-1.63 (m, 2H). LCMS m/z 338.2 (M + H)⁺ (ES⁺). From Intermediate R1, step D |

### Intermediate R27: 4-(4-Isocyanato-6-methyl-2,3-dihydro-1H-inden-5-yl)-2-((1-methylpiperidin-4-yl)oxy)pyridine

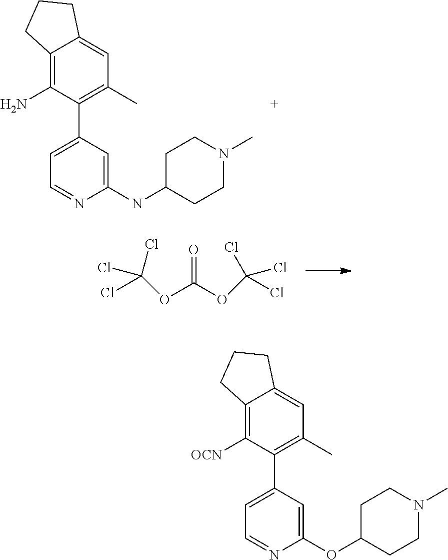

Triphosgene (0.077 g, 0.260 mmol) in THF (1 mL) was added dropwise to an ice-cooled solution of 6-methyl-5-(2-((1-methylpiperidin-4-yl)oxy)pyridin-4-yl)-2,3-dihydro-1H-inden-4-amine (Intermediate R26) (0.129 g, 0.4 mmol) and Et₃N (0.112 mL, 0.800 mmol) in THF (5 mL), and stirred at room temperature for 3 hours. The reaction mixture was filtered, washed with THF, concentrated in vacuo and dried azeotropically with toluene (3×1 mL). The crude product was used without further purification.

The following intermediate was prepared according to the general procedure of Intermediate R27:

| Int. | Structure and name | Characterisation and procedure |
|---|---|---|
| R28 | 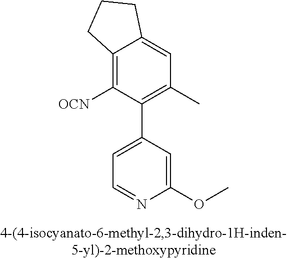 | From Intermediate R25 |

### Intermediate R29: 5-(2-Ethoxypyridin-4-yl)-6-methyl-2,3-dihydro-1H-inden-4-amine

### Step A: 5-(2-Fluoropyridin-4-yl)-6-methyl-2,3-dihydro-1H-inden-4-amine

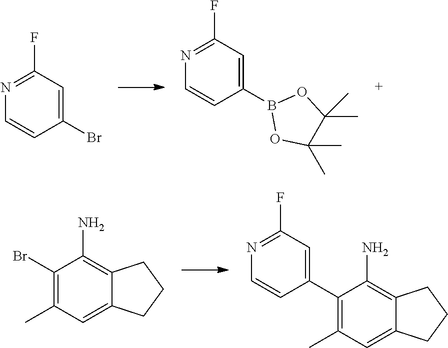 [SMILES: Fc1cc(Br)ccn1 ; CC1(C)OB(c2ccnc(F)c2)OC1(C)C ; Cc1cc2c(c(N)c1Br)CCC2 ; Cc1cc2c(c(N)c1-c1ccnc(F)c1)CCC2]

A solution of 4-bromo-2-fluoropyridine (1.170 g, 6.65 mmol), KOAc (2.60 g, 26.5 mmol), B₂Pin₂ (1.685 g, 6.63 mmol) and PdCl₂(dppe-CH₂Cl₂ adduct (0.271 g, 0.332 mmol) in 1,4-dioxane (20 mL) was heated at 100° C. for 2 hours under N₂. Then the reaction mixture was cooled to room temperature and a solution 5-bromo-6-methyl-2,3-dihydro-1H-inden-4-amine (Intermediate R1) (1.5 g, 6.63 mmol) in 1,4-dioxane (5 mL) was added, followed by a solution of K₂CO₃ (3.67 g, 26.5 mmol) in water (2.5 mL). The reaction mixture was heated at 100° C. for 2 hours, diluted with EtOAc (75 mL), and washed with water (100 mL) and brine (100 mL). The organic phase was separated, dried (MgSO₄) and evaporated in vacuo. The crude product was purified by flash chromatography (0-50% EtOAc/isohexane) to afford the title compound (940 mg, 55%) as a white solid.

¹H NMR (CDCl₃) δ 8.32 (d, J=5.0 Hz, 1H), 7.12 (dt, J=5.2, 1.6 Hz, 1H), 6.88 (s, 1H), 6.66 (s, 1H), 3.36 (s, 2H), 2.93 (t, J=7.5 Hz, 2H), 2.72 (t, J=7.4 Hz, 2H), 2.14 (p, J=7.5 Hz, 2H), 2.00 (s, 3H).

LCMS m/z 243.2 (M+H)⁺(ES⁺).

### Step B: 5-(2-Ethoxypyridin-4-yl)-6-methyl-2,3-dihydro-1H-inden-4-amine

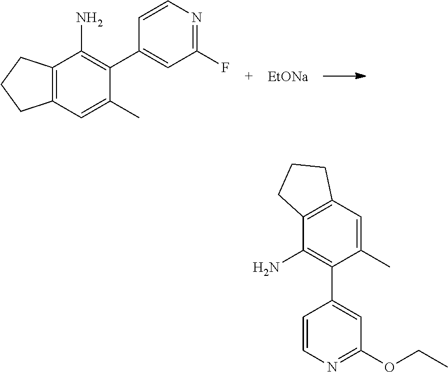 [SMILES: "EtONa" ; Cc1cc2c(c(N)c1-c1ccnc(F)c1)CCC2 ; CCOc1cc(-c2c(C)cc3c(c2N)CCC3)ccn1]

5-(2-Fluoropyridin-4-yl)-6-methyl-2,3-dihydro-1H-inden-4-amine (100 mg, 0.413 mmol) was dissolved in THF (2 mL). EtONa (42 mg, 0.617 mmol) was added, and the reaction mixture was stirred at room temperature for 18 hours. Additional EtONa (42 mg, 0.617 mmol) was added and the reaction mixture stirred for 4 hours. Then the reaction mixture was partitioned between EtOAc (20 mL) and water (10 mL). The organic layer was separated, washed with water (10 mL), dried (phase separator) and concentrated in vacuo to afford the title compound (121 mg, quantitative yield).

¹H NMR (DMSO-d6) δ 8.21 (d, J=5.2 Hz, 1H), 6.74 (dd, J=5.2, 1.4 Hz, 1H), 6.54 (s, 1H), 6.45 (s, 1H), 4.34 (q, J=7.0 Hz, 2H), 4.14 (s, 2H), 2.78 (t, J=7.5 Hz, 2H), 2.64 (t, J=7.3 Hz, 2H), 1.99 (p, J=7.5 Hz, 2H), 1.88 (s, 3H), 1.35 (t, J=7.1 Hz, 3H).

LCMS m/z 269.2 (M+H)+(ES+).

The following intermediate was prepared according to the general procedure of Intermediate R29:

| Int. | Structure and name | Characterisation and procedure |
|---|---|---|
| R30 | 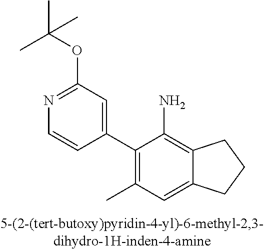 | ¹H NMR (DMSO-d6) δ 8.19 (dd, J = 5.1, 0.7 Hz, 1H), 6.70 (dd, J = 5.1, 1.4 Hz, 1H), 6.45 (s, 1H), 6.42 (dd, J = 1.4, 0.7 Hz, 1H), 4.11 (s, 2H), 2.78 (t, J = 7.5 Hz, 2H), 2.64 (t, J = 7.3 Hz, 2H), 2.03-1.96 (m, 2H), 1.88 (s, 3H), 1.58 (s, 9H). LCMS m/z 297.3 (M + H)⁺ (ES⁺). From Intermediate R29, step A |

### Intermediate R31: 5-(2-(Cyclohexyloxy)pyridin-4-yl)-6-methyl-2,3-dihydro-1H-inden-4-amine

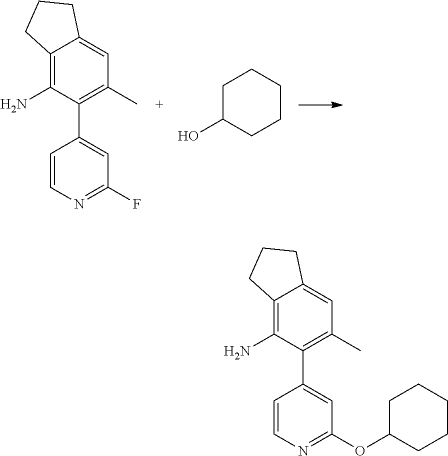 [SMILES: Cc1cc2c(c(N)c1-c1ccnc(F)c1)CCC2 ; OC1CCCCC1 ; Cc1cc2c(c(N)c1-c1ccnc(OC3CCCCC3)c1)CCC2]

KO^{t}Bu (0.132 g, 1.176 mmol) was added to cyclohexanol (0.163 mL, 1.568 mmol) in THF (3 mL). The reaction mixture was stirred at room temperature for 1 hour and then cooled to 0° C. 5-(2-Fluoropyridin-4-yl)-6-methyl-2,3-dihydro-1H-inden-4-amine (Intermediate R29, step A) (0.200 g, 0.784 mmol) was added and the reaction mixture was stirred at room temperature for 18 hours. Then the reaction mixture was partitioned between EtOAc (20 mL) and water (10 mL). The organic layer was washed with water (10 mL), dried (phase separator) and concentrated in vacuo. The crude product was purified by flash chromatography (0-25% EtOAc/isohexane) to afford the title compound (0.177 g, 61%) as a thick colourless oil.

¹H NMR (DMSO-d6) δ 8.19 (d, J=5.1 Hz, 1H), 6.71 (dd, J=5.1, 1.4 Hz, 1H), 6.49 (s, 1H), 6.44 (s, 1H), 5.04-4.96 (m, 1H), 4.12 (s, ²H), 2.77 (t, J=7.5 Hz, 2H), 2.64 (t, J=7.3 Hz, 2H), 2.04-1.94 (m, 4H), 1.88 (s, 3H), 1.78-1.69 (m, 2H), 1.59-1.52 (m, 1H), 1.51-1.33 (m, 4H), 1.30-1.22 (m, 1H).

LCMS m/z 323.3 (M+H)⁺(ES⁺).

The following intermediate was prepared according to the general procedure of Intermediate R31:

| Int. | Structure and name | Characterisation and procedure |
|---|---|---|
| R32 | 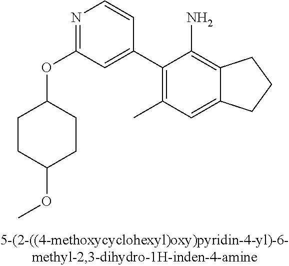 | ¹H NMR (DMSO-d6) δ 8.20 (t, J = 4.8 Hz, 1H), 6.73-6.71 (m, 1H), 6.51-6.50 (m, 1H), 6.45 (s, 1H), 5.12-5.06 (m, 0.5H), 5.05-4.98 (m, 0.5H), 4.13 (s, 2H), 3.25 (s, 1.5H), 3.24 (s, 1.5H), 2.78 (t, J = 7.5 Hz, 2H), 2.64 (t, J = 7.3 Hz, 2H), 2.13- 2.04 (m, 1H), 2.05-1.94 (m, 4H), 1.88 (d, J = 3.0 Hz, 3H) 1.84-1.69 (m, 3H), 1.69- 1.58 (m, 1H), 1.54-1.44 (m, 1H), 1.41- 1.30 (m, 1H). 50:50 mixture of cis/trans ring. LCMS m/z 353.3 (M + H)⁺ (ES⁺). From Intermediate R29, step A |

### Intermediate R33: 4-Fluoro-6-isopropyl-2-(2-methoxypyridin-4-yl)-3-methylaniline

### Step A: 4-Fluoro-5-methyl-2-(prop-1-en-2-yl)aniline

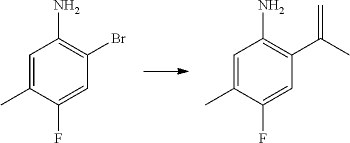

A mixture of 2-bromo-4-fluoro-5-methylaniline (10.00 g, 49.0 mmol), 4,4,5,5- tetramethyl-2-(prop-1-en-2-yl)-1,3,2-dioxaborolane (11 mL, 58.8 mmol), Pd(OAc)₂ (440 mg, 1.960 mmol), tricyclohexylphosphine (21.2 g, 76 mmol) and K₃PO₄ (28.1 g, 132 mmol) in dioxane (120 mL) and water (30 mL) was degassed with N₂. Then the reaction mixture was heated at 100° C. for 18 hours. Solvent was evaporated and the io residue partitioned between isohexane (500 mL) and water (300 mL). The organic layer was washed with water (200 mL), dried (phase separator) and concentrated in vacuo. The crude product was purified by flash chromatography on silica gel (0-40% EtOAc/isohexane) to afford the title compound (9.09 g, 99%) as a brown oil.

¹H NMR (DMSO-d₆) δ 6.70 (d, J=10.6 Hz, 1H), 6.52 (d, J=7.3 Hz, 1H), 5.24-5.20 (m, 1H), 5.01-4.98 (m, 1H), 4.59 (br s, 2H), 2.09 (s, 3H), 1.98 (s, 3H).

### Step B: 4-Fluoro-2-isopropyl-5-methylaniline

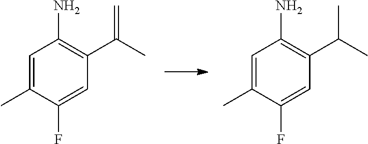

A mixture of 4-fluoro-5-methyl-2-(prop-1-en-2-yl)aniline (13.33 g, 81 mmol) and 5% Pd/C (Type 87L, 58.5% moisture) (1.66 g, 0.324 mmol) in EtOAc (145 mL) was hydrogenated at 3 bar for 16 hours. Then the reaction mixture was filtered through Celite® and concentrated in vacuo to afford the title compound (11.95 g, 79%) as a dark green oil.

¹H NMR (DMSO-d₆) δ 6.74 (d, J=11.4 Hz, 1H), 6.50 (d, J=7.3 Hz, 1H), 5.07 (s, 2H), 2.92 (hept, J=6.7 Hz, 1H), 2.08 (s, 3H), 1.11 (d, J=6.8 Hz, 6H).

LCMS m/z 168.1 (M+H)+(ES+).

### Step C: 2-Bromo-4-fluoro-6-isopropyl-3-methylaniline

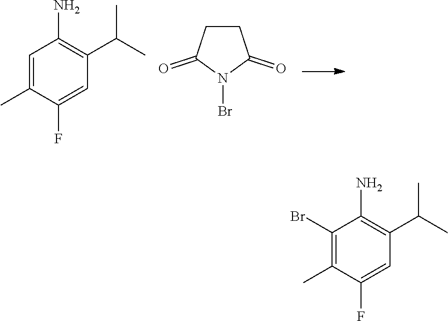

NBS (12.08 g, 67.9 mmol) was added to a solution of 4-fluoro-2-isopropyl-5-methylaniline (11.95 g, 67.9 mmol) in DCM (180 mL). The reaction mixture was stirred at room temperature for 20 minutes, then washed with water (200 mL) and io% aqueous Na₂S₂O₃ (200 mL), dried (phase separator) and concentrated in vacuo to afford crude product (14.6 g). 5 g of the crude product was purified by flash chromatography on silica gel (0-20% EtOAc/isohexane) to afford the title compound (3.26 g, 19%) as a red-orange oil.

¹H NMR (DMSO-d₆) δ 6.89 (d, J=11.0 Hz, 1H), 4.87 (s, 2H), 3.06 (sept, J=6.7 Hz, 1H), 2.20 (d, J=2.4 Hz, 3H), 1.14 (d, J=6.7 Hz, 6H).

LCMS m/z 246.1/248.1 (M+H)⁺(ES⁺).

### Step D: 4-Fluoro-6-isopropyl-2-(2-methoxypyridin-4-yl)-3-methylaniline

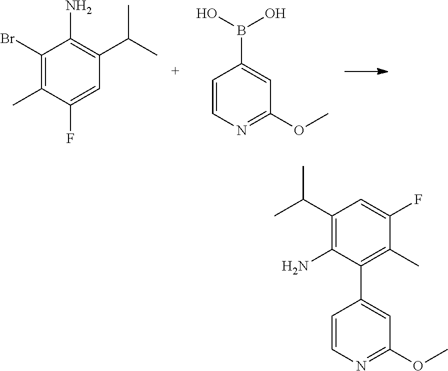

(2-Methoxypyridin-4-yl)boronic acid (200 mg, 1.308 mmol), 2-bromo-4-fluoro-6-isopropyl-3-methylaniline (322 mg, 1.308 mmol), potassium carbonate (723 mg, 5.23 mmol) and PdCl₂(dppf). DCM (53 mg, 0.065 mmol) were dissolved in 1,4-dioxane (6 mL) and water (3 mL). The reaction mixture was degassed (N₂, 5 minutes) and evacuated and backfilled with N₂ (×₃). Then the reaction mixture was stirred at 100° C. for 3 hours. The reaction mixture was diluted with EtOAc (20 mL) and washed with brine (2×20 mL). The organic extract was dried (phase separator) and concentrated in vacuo. The crude product was purified by flash chromatography on silica gel (0-100% EtOAc/isohexane) to afford the title compound (276 mg, 73%) as a brown oil.

¹H NMR (DMSO-d₆) δ 8.28 (d, J=5.1 Hz, 1H), 6.88 (d, J=11.3 Hz, 1H), 6.81 (dd, J=5.2, 1.4 Hz, 1H), 6.65 (s, 1H), 3.99 (s, 2H), 3.90 (s, 3H), 3.03-2.96 (m, 1H), 1.79 (d, J=2.1 Hz, 3H), 1.15 (d, J=6.7 Hz, 6H).

LCMS (m/z 275.1 (M+H)+(ES+).

The following intermediates were prepared according to the general procedure of Intermediate R33:

| Int. | Structure and name | Characterisation and procedure |
|---|---|---|
| R34 | 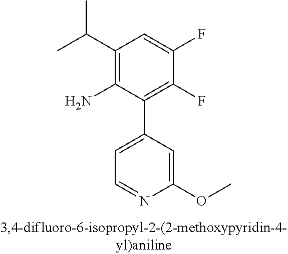 | ¹H NMR (DMSO-d6) δ 8.28 (d, J = 5.3 Hz, 1H), 7.11 (dd, J = 12.3, 9.0 Hz, 1H), 6.97- 6.92 (m, 1H), 6.80 (s, 1H), 4.58 (s, 2H), 3.90 (s, 3H), 3.02 (sept, J = 6.8 Hz, 1H), 1.16 (d, J = 6.8 Hz, 6H). LCMS m/z 279.2 (M + H)⁺ (ES⁺). |
| R35 | 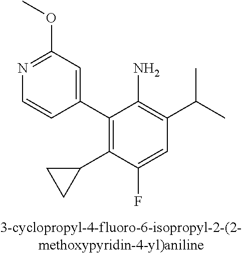 | ¹H NMR (DMSO-d6) δ 8.26 (d, J = 5.1 Hz, 1H), 6.88 (dd, J = 5.2, 1.3 Hz, 1H), 6.82 (d, J = 12.7 Hz, 1H), 6.70 (s, 1H), 3.89 (s, 3H), 4.01 (s, 2H), 2.98 (app p, J = 6.7 Hz, 1H), 1.43-1.33 (m, 1H), 1.15 (d, J = 6.7 Hz, 6H), 0.57-0.41 (m, 4H). LCMS m/z 301.1 (M + H)⁺ (ES⁺). From Intermediate R2 |
| R36 | 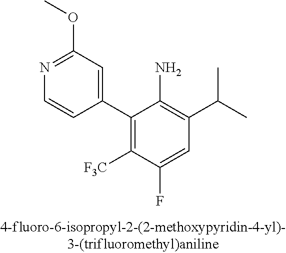 | LCMS m/z 329.1 (M + H)⁺ (ES⁺). |
| R37 | 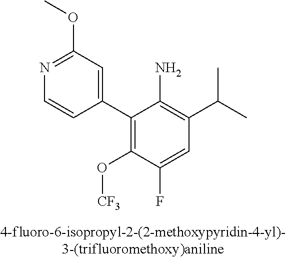 | ¹H NMR (DMSO-d6) δ 8.29 (d, J = 5.2 Hz, 1H), 7.18 (d, J = 12.0 Hz, 1H), 6.89 (dd, J = 5.2, 1.4 Hz, 1H), 6.73 (s, 1H), 4.58 (s, 2H), 3.90 (s, 3H), 3.05 (sept, J = 6.8 Hz, 1H), 1.18 (d, J = 6.7 Hz, 6H). LCMS m/z 345.2 (M + H)⁺ (ES⁺). From Intermediate R3 |
| R38 | 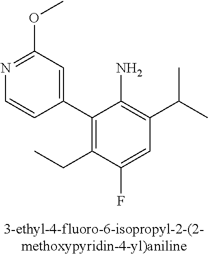 | ¹H NMR (DMSO-d6) δ 8.28 (d, J = 5.1 Hz, 1H), 6.88 (d, J = 11.6 Hz, 1H), 6.83 (dd, J = 5.2, 1.4 Hz, 1H), 6.66 (t, J = 1.1 Hz, 1H), 3.97 (s, 2H), 3.91 (s, 3H), 2.98 (hept, J = 6.6 Hz, 1H), 2.26-2.13 (m, 2H), 1.16 (dd, J = 6.8, 2.0 Hz, 6H), 0.90 (t, J = 7.5 Hz, 3H). LCMS m/z 289.1 (M + H)⁺ (ES⁺). From Intermediate R4 |

### Intermediate R39: 4-Isopropyl-2-methyl-1-(pyridin-4-yl)-1H-imidazol-5-amine

### Step A: 2-Methyl-1-((2-(trimethylsilyl)ethoxy)methyl)-1H-imidazole

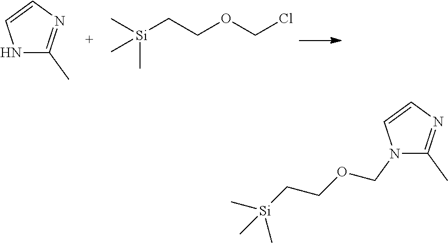

To a solution of NaH (9.74 g, 243.59 mmol, 60 wt % in mineral oil, 1 eq) in DMF (200 mL) was added in portions 2-methyl-1H-imidazole (20 g, ²43.59 mmol, 1 eq) at 0° C. The reaction mixture was stirred at 0° C. for 3o minutes. Then (2-(chloromethoxy)ethyl) trimethylsilane (48.73 g, 292.31 mmol, 1.2 eq) was added. The resulting mixture was stirred at 0° C. for 2 hours. The reaction mixture was quenched with ice-water (300 mL), diluted with ethyl acetate (1 L), and washed with saturated aqueous NH₄Cl solution (3×300 mL) and brine (3×300 mL). The organic layers were dried over anhydrous Na₂SO₄, filtered and concentrated in vacuo. The residue was purified by column chromatography (SiO₂, petroleum ether: ethyl acetate, 5:1 to 1:1) to give the title compound (40 g, 76% yield, 98% purity on LCMS) as a yellow oil.

¹H NMR (400 MHz, CDCl₃) δ 6.90 (s, 2H), 5.18 (s, 2H), 3.47 (t, 2H), 2.43 (s, 3H), 0.89 (t, 2H) and 0.01 (s, 9H).

LCMS: m/z 213.0 (M+H)⁺(ES⁺).

### Step B: 4-Bromo-2-methyl-1-((2-(trimethylsilyl)ethoxy)methyl)-1H-imidazole

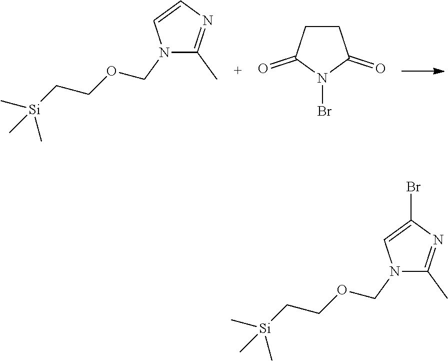

To a solution of 2-methyl-1-((2-(trimethylsilyl)ethoxy)methyl)-1H-imidazole (20 g, 94.18 mmol, 1 eq) in DMF (200 mL) was added NBS (16.76 g, 94.18 mmol, 1 eq) at −20° C. Then the reaction mixture was stirred at −20° C. for 2 hours. The reaction mixture was quenched with saturated aqueous Na₂SO₃solution (100 mL), diluted with EtOAc (200 mL), and washed with saturated aqueous NH₄Cl solution (3×100 mL) and brine (3×100 mL). The organic layers were dried over anhydrous Na₂SO₄, filtered and concentrated in vacuo. The residue was purified by column chromatography (SiO₂, petroleum ether: ethyl acetate, 10:1 to 5:1) to give the title compound (13.5 g, 41% yield, 84% purity on LCMS) as a yellow oil.

¹H NMR (400 MHz, CDCl₃) δ 6.88 (s, 1H), 5.25 (s, 2H), 3.55 (t, 2H), 2.42 (s, 3H), 0.91 (t, 2H) and 0.02 (s, 9H).

LCMS: m/z 292.9 (M+H)⁺(ES⁺).

### Step C: 2-Methyl-4-(prop-1-en-2-yl)-1-((2-(trimethylsilyl)ethoxy)methyl)-1H-imidazole

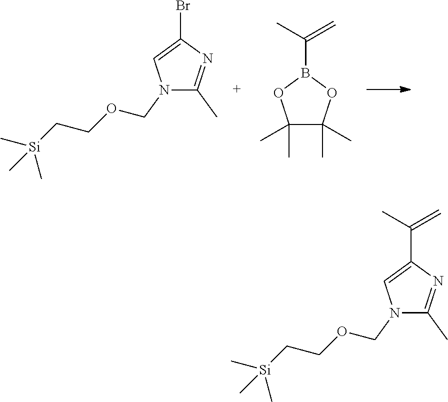

A solution of 4-bromo-2-methyl-1-((2-(trimethylsilyl)ethoxy)methyl)-1H-imidazole (10 g, 28.84 mmol, 1 eq), 4,4,5,5-tetramethyl-2-(prop-1-en-2-yl)-1,3,2-dioxaborolane (5.33 g, 31.72 mmol, 1.1 eq), Pd(dppf)Cl₂ (1.06 g, 1.44 mmol, 0.05 eq) and Na₂CO₃ (6.11 g, 57.68 mmol, 2 eq) in dioxane (100 mL) and H₂O (20 mL) was stirred at 100° C. for 12 hours under N₂. The reaction mixture was diluted with water (100 mL), and then is extracted with ethyl acetate (3×100 mL). The organic layers were dried over anhydrous Na₂SO₄, filtered and concentrated in vacuo. The residue was purified by column chromatography (SiO₂, petroleum ether: ethyl acetate, 5:1 to 1:1) to give the title compound (7 g, 96%) as a yellow oil.

¹H NMR (400 MHz, CDCl₃) δ 6.88 (s, 1H), 5.23 (s, 2H), 5.20 (s, 1H), 5.14 (s, 1H), 3.52 (t, 2H), 2.48 (s, 3H), 2.08 (s, 3H), 0.93 (t, 2H) and 0.01 (s, 9H).

LCMS: m/z 253.0 (M+H)⁺(ES⁺).

### Step D: 4-Isopropyl-2-methyl-1-((2-(trimethylsilyl)ethoxy)methyl)-1H-imidazole

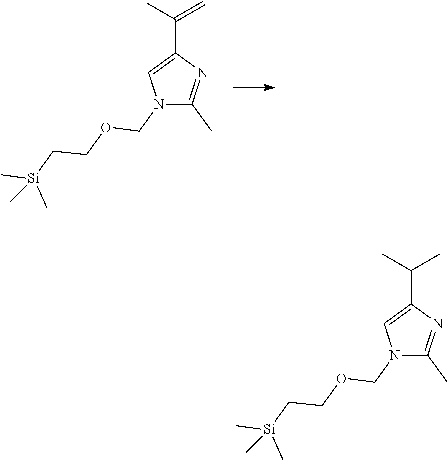

To a solution of 2-Methyl-4-(prop-1-en-2-yl)-1-((2-(trimethylsilyl)ethoxy)methyl)-1H-imidazole (7.18 g, 28.44 mmol, 1 eq) in MeOH (100 mL) was added Pd/C (700 mg, 10 wt % loading on activated carbon) under N₂. The suspension was degassed in vacuo and purged with H₂ several times. The mixture was stirred at 25° C. for 12 hours under H₂ (15 psi). Then the reaction mixture was filtered and the filtrate was concentrated in vacuo to give the title compound (8 g, 99% yield, 90% purity on LCMS) as a yellow oil.

¹H NMR (400 MHz, CDCl₃) δ 6.66 (s, 1H), 5.15 (s, 2H), 3.49 (t, 2H), 2.95-2.84 (m, 1H), 2.43 (s, 3H), 1.26 (d, 6H), 0.91 (t, 2H) and 0.02 (s, 9H).

LCMS: m/z 255.2 (M+H)⁺(ES⁺).

### Step E: 4-Isopropyl-2-methyl-1H-imidazole

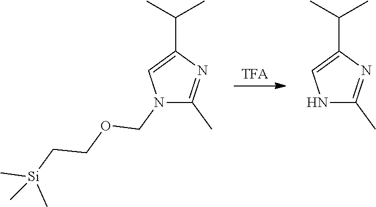

To a solution of 4-isopropyl-2-methyl-1-((2-(trimethylsilyl)ethoxy)methyl)-1H-imidazole (8 g, ₃1.44 mmol, 1 eq) in DCM (8o mL) was added TFA (123.20 g, 1.08 mol, 34.37 eq) at 25° C. Then the mixture was stirred at 25° C. for 12 hours. The reaction mixture was quenched with ice-water (io mL) and saturated aqueous NaHCO₃ solution (300 mL). The mixture was extracted with ethyl acetate (2×100 mL). The combined organic layers were washed with brine (2×200 mL), dried over anhydrous Na₂SO₄, filtered and concentrated in vacuo. The residue was purified by column chromatography (SiO₂, ethyl acetate: methanol, 1:0 to 20:1) to give the title compound (3.7 g, 95%) as a yellow oil.

¹H NMR (400 MHz, CDCl₃) δ 6.71 (s, 1H), 2.99-2.93 (m, 1H), 2.53 (s, 3H) and 1.27 (d, 6H).

LCMS: m/z 125.3 (M+H)⁺(ES⁺).

### Step F: 4-(4-Isopropyl-2-methyl-1H-imidazol-1-yl)pyridine

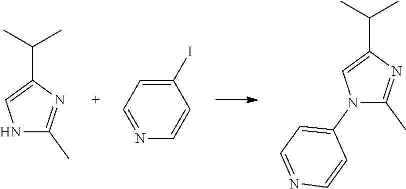

To a solution of 4-isopropyl-2-methyl-1H-imidazole (1.4 g, 11.27 mmol, 1 eq) and 4-iodopyridine (1.85 g, 9.02 mmol, 0.8 eq) in DMF (14 mL) was added with Cu₂O (81 mg, 563.68 μmol, 0.05 eq) and Cs₂CO₃ (7.35 g, 22.55 mmol, 2 eq). The reaction mixture was stirred at 100° C. for 15 hours. Then the reaction mixture was diluted with ethyl acetate (50 mL), and washed with saturated aqueous NH₄Cl solution (3×30 mL) and brine (3×30 mL). The organic layers were dried over anhydrous Na₂SO₄, filtered and concentrated in vacuo. The residue was purified by column chromatography (SiO₂, petroleum ether: ethyl acetate, 5:1 to 0:1) to give the title compound (600 mg, 26% yield, 97% purity on LCMS) as a yellow solid.

¹H NMR (400 MHz, CDCl₃) δ 8.73 (dd, 2H), 7.27 (dd, 2H), 6.77 (s, 1H), 2.93-2.86 (m, 1H), 2.48 (s, 3H) and 1.29 (d, 6H).

LCMS: m/z 202.0 (M+H)⁺(ES⁺).

### Step G: 4-(4-Isopropyl-2-methyl-5-nitro-1H-imidazol-1-yl)pyridine

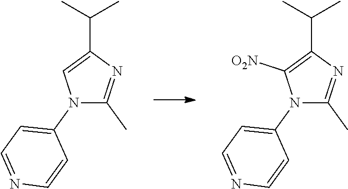 [SMILES: Cc1nc(C(C)C)cn1-c1ccncc1 ; Cc1nc(C(C)C)c([N+](=O)[O-])n1-c1ccncc1]

To a solution of 4-(4-isopropyl-2-methyl-1H-imidazol-1-yl)pyridine (400 mg, 1.93 mmol, 1 eq) in H₂SO₄ (71.33 mmol, 3.88 mL, 98% purity in solution, 37 eq) was added with HNO₃ (5.78 mmol, 400 μL, 65% purity in aqueous solution, 3 eq) at 0° C. Then the reaction mixture was stirred at 25° C. for 12 hours. The reaction mixture was quenched with ice-water (20 mL), and adjusted to pH=8˜9 with saturated aqueous NaHCO₃ solution. The mixture was extracted with ethyl acetate (3×20 mL). The organic layers were dried over anhydrous Na₂SO₄, filtered and concentrated in vacuo. The yellow solid was purified by column chromatography (SiO₂, petroleum ether: ethyl acetate, 2:1 to 1:1) to give the title compound (400 mg, 84%) as a yellow solid.

¹H NMR (400 MHz, CDCl₃) δ 8.83 (d, 2H), 7.22 (d, 2H), 3.75-3.69 (m, 1H), 2.25 (s, 3H) and 1.36 (d, 6H).

LCMS: m/z 247.1 (M+H)⁺(ES⁺).

### Step H: 4-Isopropyl-2-methyl-1-(pyridin-4-yl)-1H-imidazol-5-amine

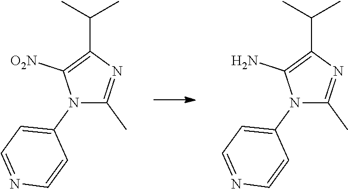 [SMILES: Cc1nc(C(C)C)c([N+](=O)[O-])n1-c1ccncc1 ; Cc1nc(C(C)C)c(N)n1-c1ccncc1]

A mixture of 4-(4-isopropyl-2-methyl-5-nitro-1H-imidazol-1-yl)pyridine (400 mg, 1.62 mmol, 1 eq) and Pd/C (40 mg, 10 wt % loading on activated carbon) in MeOH (20 mL) was hydrogenated at 20° C. for 1 hour under H₂ (15 psi). Then the reaction mixture was filtered, and the filtrate was concentrated in vacuo. The residue was dissolved in THF (10 mL), and adjusted to pH−3˜4 with 4M HCl/dioxane. The resulting mixture was concentrated in vacuo to give the title compound (400 mg, 97%, HCl salt) as a yellow solid, which was used in the next step without further purification.

¹H NMR (400 MHz, DMSO-d₆) δ 15.02 (s, 1H), 8.99 (d, 2H), 7.90 (d, 2H), 3.25-3.15 (m, 1H), 2.45 (s, 3H) and 1.27 (d, 6H).

LCMS: m/z 217.1 (M+H)⁺(ES⁺).

### Intermediate R40: 4-Isopropyl-1-(2-methoxypyridin-4-yl)-2-methyl-1H-imidazol-5-amine

### Step A: 4-(4-Isopropyl-2-methyl-1H-imidazol-1-yl)-2-methoxypyridine

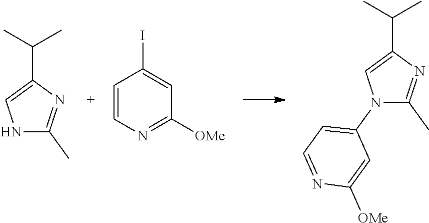

A reaction mixture of 4-isopropyl-2-methyl-1H-imidazole (Intermediate R39, step E) (1 g, 6.44 mmol, 1 eq), 4-iodo-2-methoxypyridine (1.51 g, 6.44 mmol, 1 eq), Cu₂O (922 mg, 6.44 mmol, 1 eq) and Cs₂CO₃ (4.20 g, 12.88 mmol, 2 eq) in DMF (10 mL) was stirred at 100° C. for 12 hours. Then the reaction mixture was filtered. The filtrate was poured into water (20 mL) and extracted with ethyl acetate (3×30 mL). The organic layers were dried over anhydrous Na₂SO₄, filtered, and concentrated in vacuo. The residue was purified by column chromatography (SiO₂, petroleum ether: ethyl acetate, 5:1 to 1:1) to give the title compound (700 mg, 47%) as a yellow oil.

¹H NMR (400 MHz, CD₃OD) δ 8.25 (d, 1H), 7.03 (d, 1H), 6.98 (s, 1H), 6.84 (s, 1H), 3.96 (s, 3H), 2.87-2.80 (m, 1H), 2.46 (s, 3H) and 1.26 (d, 6H).

LCMS: m/z 232.2 (M+H)⁺(ES⁺).

### Step B: 4-(4-Isopropyl-2-methyl-5-nitro-1H-imidazol-1-yl)-2-methoxypyridine

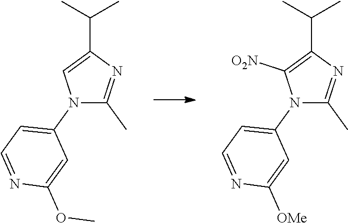 [SMILES: COc1cc(-n2cc(C(C)C)nc2C)ccn1 ; COc1cc(-n2c(C)nc(C(C)C)c2[N+](=O)[O-])ccn1]

To a solution of 4-(4-isopropyl-2-methyl-1H-imidazol-1-yl)-2-methoxypyridine (0.7 g, 3.03 mmol, 1 eq) in H₂SO₄ (12.88 g, 98 wt % in aqueous solution, 131.32 mmol, 43.39 eq) was added with HNO₃ (829 mg, 9.08 mmol, 69 wt % in aqueous solution, 3 eq) at 0° C. Then the reaction mixture was stirred at 25° C. for 2 hours. The reaction mixture was poured into ice-water (40 mL), and adjust to pH=8˜9 with NaOH solid. Then the mixture was extracted with ethyl acetate (3×50 mL). The organic layers were dried over anhydrous Na₂SO₄, filtered, and concentrated in vacuo. The residue was purified by column chromatography (SiO₂, petroleum ether: ethyl acetate, 5:1 to 3:1) to give the title compound (600 mg, 67% yield, 94% purity on LCMS) as a yellow solid.

¹H NMR (400 MHz, CD₃OD) δ 8.33 (d, 1H), 6.99 (dd, 1H), 6.90 (d, 1H), 4.00 (s, 3H), 3.76-3.68 (m, 1H), 2.25 (s, 3H) and 1.34 (d, 6H).

LCMS: m/z 277.0 (M+H)⁺(ES⁺).

### Step C: 4-Isopropyl-1-(2-methoxypyridin-4-yl)-2-methyl-1H-imidazol-5-amine

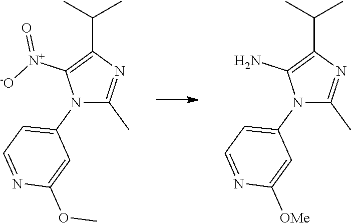

To a solution of 4-(4-isopropyl-2-methyl-5-nitro-1H-imidazol-1-yl)-2-methoxypyridine (200 mg, 723.88 μmol, 1 eq) in MeOH (5 mL) was added Pd/C (20 mg, 10 wt % loading on the activated carbon) under N₂ atmosphere. The suspension was degassed under vacuum and purged with H₂ several times. The mixture was stirred at 25° C. for 2 hours under H₂ (15 psi). Then the reaction mixture was filtered, and the filtrate was concentrated in vacuo to give the title compound (178 mg, 100%) as a yellow oil, which was used directly in the next step.

¹H NMR (400 MHz, CDCl₃) δ 8.30 (d, 1H), 6.81 (dd, 1H), 6.65 (d, 1H), 4.01 (s, 3H), 3.65-3.57 (m, 1H), 2.26 (s, 3H) and 1.37 (d, 6H).

### Intermediate R41

### Phenyl (6-methyl-5-(2-((1-methylpiperidin-4-yl)oxy)pyridin-4-yl)-2,3-dihydro-1H-inden-4-yl)carbamate

### Step A: 4-Bromo-2-((1-methylpiperidin-4-yl)oxy)pyridine

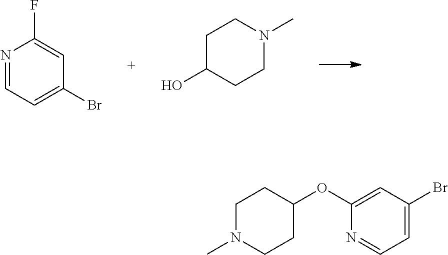

To a solution of KOtBu (41.13 g, 366.51 mmol, 1.5 eq) in THF (500 mL) was added 1-methylpiperidin-4-ol (33.77 g, 293.20 mmol, 1.2 eq) at 20° C. The reaction mixture was stirred for 1 hour. Then 4-bromo-2-fluoropyridine (43 g, 244.34 mmol, 1 eq) was added at 0° C. The reaction mixture was stirred at 20° C. for 12 hours, and then poured into water (500 mL). The aqueous phase was extracted with ethyl acetate (2×500 mL). The combined organic phases were washed with brine (2×500 mL), dried over anhydrous Na₂SO₄, filtered and concentrated in vacuo. The residue was purified by column chromatography (SiO₂, with 0.1% NH₃.H₂O, DCM: methanol 1:0 to 10:1) to give the title is compound (61 g, 92%) as a yellow solid.

¹H NMR (400 MHz, DMSO-d₆) δ 8.05 (d, 1H), 7.18 (dd, 1H), 7.06 (s, 1H), 4.98-4.93 (m, 1H), 2.62-2.59 (m, 2H), 2.16-2.11 (m, 5H) 1.94-1.91 (m, 2H) and 1.66-1.62 (m, 2H)

LCMS: m/z 273.0 (M+H)⁺(ES⁺).

### Step B: 2-((1-Methylpiperidin-4-yl)oxy)-4-(4,4,5,5-tetramethyl-1,3,2-dioxaborolan-2-yl)pyridine

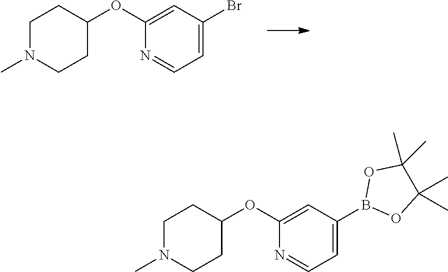

To a mixture of 4-bromo-2-((1-methylpiperidin-4-yl)oxy)pyridine (20 g, 73.76 mmol, 1 eq) and 4,4,5,5-tetramethyl-2-(4,4,5,5-tetramethyl-1,3,2-dioxaborolan-2-yl)-1,3,2-dioxaborolane (24.35 g, 95.89 mmol, 1.3 eq) in dioxane (200 mL) was added PdCl₂(dppf) (3.24 g, 4.43 mmol, 0.06 eq) and KOAc (34.24 g, 348.88 mmol, 4.73 eq) in one portion under N₂. Then the reaction mixture was heated to 100° C. for 2 hours. The reaction mixture was concentrated in vacuo. The residue was purified by reserved phase flash chromatography (0.1% NH₃.H₂O-MeCN) to give the title compound (22.5 g, 96%) as a brown oil.

¹H NMR (400 MHz, DMSO-d₆) δ 8.17-8.12 (m, 1H), 7.08-7.03 (m, 1H), 6.93-6.88 (m, 1H), 5.05-4.90 (m, 1H), 3.92-3.86 (m, 2H), 2.73-2.66 (m, 2H), 2.22 (s, 3H), 1.95-1.90 (m, 2H), 1.69-1.63 (m, 2H) and 1.06 (s, 12H).

LCMS: m/z 319.0 (M+H)⁺(ES⁺).

### Step C

### 6-Methyl-5-(2-((1-methylpiperidin-4-yl)oxy)pyridin-4-yl)-2,3-dihydro-1H-inden-4-amine

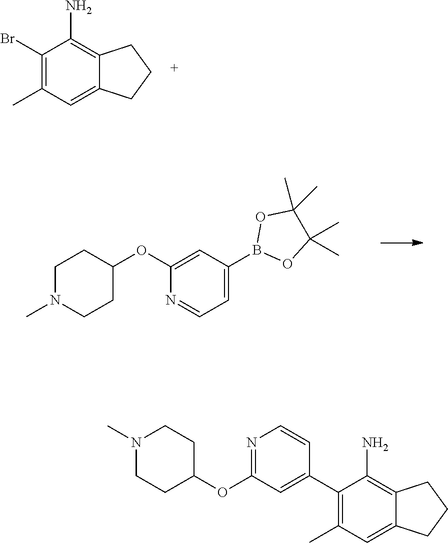

To a mixture of 5-bromo-6-methyl-2,3-dihydro-1H-inden-4-amine (Intermediate R1) (40 g, 176.90 mmol, 1 eq) and 2-((1-methylpiperidin-4-yl)oxy)-4-(4,4,5,5-tetramethyl-1,3,2-dioxaborolan-2-yl)pyridine (78.81 g, 247.66 mmol, 1.4 eq) in dioxane (500 mL) and H₂O (100 mL) was added K₂CO₃ (73.35 g, 530.71 mmol, 3 eq) and PdCl₂(dppf) (7.77 g, 10.61 mmol, 0.06 eq) in one portion under N₂. Then the reaction mixture was stirred at 100° C. for 12 hours. The reaction mixture was quenched with water (500 mL) and extracted with EtOAc (3×500 mL). The combined organic phases were washed with brine (500 mL), dried over anhydrous Na₂SO₄, filtered and concentrated in vacuo. The residue was diluted with DCM (300 mL) and extracted with HCl (3×100 mL, 3 M). The combined aqueous phases were adjusted to pH 8 with saturated aqueous Na₂CO₃solution, and then extracted with DCM (3×200 mL). The combined organic phases were washed with brine (200 mL), dried over anhydrous Na₂SO₄, filtered and concentrated in vacuo. The residue was purified by column chromatography (SiO₂, PE: EtOAc 1:0 to 5:1, then DCM: MeOH 1:0 to 10:1 with 0.1% NH₃.H₂O) to give the title compound (50 g, 80% yield, 95.6% purity on HPLC) as a brown gum.

¹H NMR (400 MHz, DMSO-d₆) δ 8.20 (d, 1H), 6.72 (dd, 1H), 6.50 (s, 1H), 6.44 (s, 1H), 5.02-4.97 (m, 1H), 4.13 (s, 2H), 2.77 (t, 2H), 2.67-2.61 (m, 4H), 2.17 (s, 3H), 2.16-2.11 (m, 2H), 2.02-1.94 (m, 4H), 1.87 (s, 3H) and 1.72-1.64 (m, 2H).

LCMS: m/z 338.2 (M+H)⁺(ES⁺).

### Step D

### Phenyl (6-methyl-5-(2-((1-methylpiperidin-4-yl)oxy)pyridin-4-yl)-2,3-dihydro-1H-inden-4-yl)carbamate

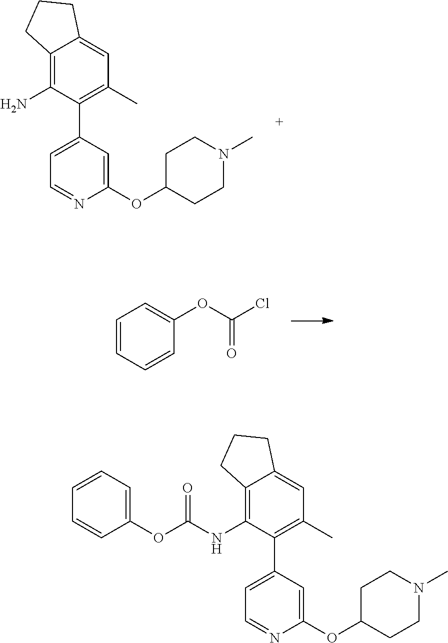 [SMILES: Cc1cc2c(c(N)c1-c1ccnc(OC3CCN(C)CC3)c1)CCC2 ; O=C(Cl)Oc1ccccc1 ; Cc1cc2c(c(NC(=O)Oc3ccccc3)c1-c1ccnc(OC3CCN(C)CC3)c1)CCC2]

To a solution of 6-methyl-5-(2-((1-methylpiperidin-4-yl)oxy)pyridin-4-yl)-2,3-dihydro-1H-inden-4-amine (1 g, 2.96 mmol, 1 eq) and phenyl carbonochloridate (463 mg, 2.96 mmol, 1 eq) in DCM (20 mL) was added TEA (300 mg, 2.96 mmol, 1 eq) at 0° C. Then the reaction mixture was stirred at 25° C. for 2 hours. The reaction mixture was concentrated in vacuo. The residue was purified by reversed phase flash io chromatography (0.1% TFA in water-MeCN) to give the title compound (350 mg, 20% yield, 95% purity on LCMS, TFA salt) as a yellow solid.

¹H NMR (400 MHz, DMSO-d₆) δ 9.70-9.60 (m, 1H), 9.19 (s, 1H), 8.25 (t, 1H), 7.36-7.34 (m, 2H), 7.23-7.16 (m, 2H), 6.90-6.81 (m, 3H), 6.68-6.62 (m, 1H), 5.36-5.19 (m, 1H), 3.39-3.14 (m, 4H), 2.97-2.91 (m, 2H), 2.87-2.79 (m, 5H), 2.38-2.33 (m, 1H), 2.27-2.16 (m, 1H), 2.06 (d, 6H) and 1.90-1.78 (m, 1H).

LCMS: m/z 458.1 (M+H)⁺(ES⁺).

### Preparation of Examples

### Example 1

### N-((6-Methyl-5-(2-methylpyridin-4-yl)-2,3-dihydro-1H-inden-4-yl)carbamoyl)methanesulfonamide

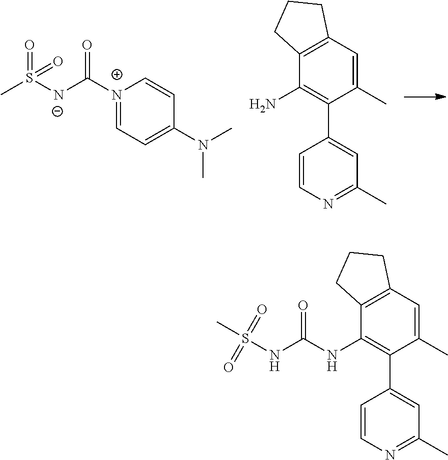

6-Methyl-5-(2-methylpyridin-4-yl)-2,3-dihydro-1H-inden-4-amine (Intermediate R9) (35 mg, 0.147 mmol) was added to a suspension of (4-(dimethylamino)pyridin-1-ium-1-carbonyl)(methylsulfonyl)amide (Intermediate L1) (36 mg, 0.148 mmol) in acetonitrile (1 mL). The reaction mixture was stirred at 60° C. for 1 hour. Volatiles were evaporated, and the crude product dissolved in DMSO (1 mL) and filtered. The crude product was purified by basic prep HPLC (10-40% MeCN in water) to afford the title compound (7 mg, 13%) as a white solid.

¹H NMR (DMSO-d6) δ 8.48 (d, J=5.1 Hz, 1H), 7.11 (s, 1H), 7.04 (s, 1H), 6.96 (d, J=5.1 Hz, 1H), 3.30 (s, 3H), 3.02 (s, 3H), 2.91 (t, J=7.4 Hz, 2H), 2.75 (t, J=7.3 Hz, 2H), 2.04-1.97 (m, 5H). Two exchangeable protons not observed.

LCMS m/z 360.2 (M+H)⁺(ES⁺).

The following examples were prepared according to the general procedure of Example 1:

| Ex. | Structure and name | Characterisation and procedure |
|---|---|---|
| 2 | 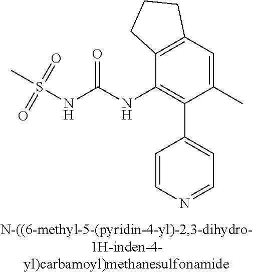 | ¹H NMR (DMSO-d6) δ 8.66-8.59 (m, 2H), 7.50 (s, 1H), 7.23-7.15 (m, 2H), 7.12 (s, 1H), 2.99 (s, 3H), 2.91 (t, J = 7.4 Hz, 2H), 2.76 (t, J = 7.5 Hz, 2H), 2.05-1.96 (m, 5H). One exchangeable proton not observed. LCMS m/z 346.4 (M + H)⁺ (ES⁺). Intermediate L1 + Intermediate R8 |
| 3 | 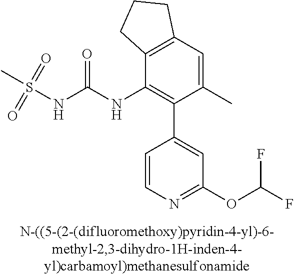 [SMILES: Cc1cc2c(c(NC(=O)NS(C)(=O)=O)c1-c1ccnc(OC(F)F)c1)CCC2] | ¹H NMR (DMSO-d6) δ 8.29 (d, J = 5.1 Hz, 1H), 7.77 (t, J = 72.9 Hz, 1H), 7.50 (s, 1H), 7.11 (s, 1H), 7.05 (dd, J = 5.1, 1.4 Hz, 1H), 6.88 (s, 1H), 2.95 (s, 3H), 2.91 (t, J = 7.4 Hz, 2H), 2.81-2.72 (m, 2H), 2.05-1.95 (m, 5H). One exchangeable proton not observed. LCMS m/z 412.3 (M + H)⁺ (ES⁺). Intermediate L1 + Intermediate R15 |
| 4 | 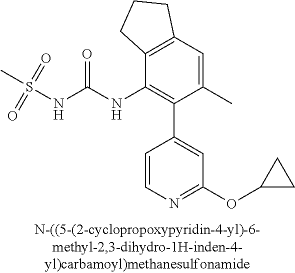 [SMILES: Cc1cc2c(c(NC(=O)NS(C)(=O)=O)c1-c1ccnc(OC3CC3)c1)CCC2] | ¹H NMR (DMSO-d6) δ 8.24 (d, J = 5.1 Hz, 1H), 7.46 (s, 1H), 7.10 (s, 1H), 6.79 (dd, J = 5.2, 1.4 Hz, 1H), 6.63 (d, J = 1.2 Hz, 1H), 4.21 (tt, J = 6.3, 3.1 Hz, 1H), 3.02 (s, 3H), 2.90 (t, J = 7.5 Hz, 2H), 2.78-2.71 (m, 2H), 2.05-1.95 (m, 5H), 0.80-0.64 (m, 4H). One exchangeable proton not observed. LCMS m/z 402.3 (M + H)⁺ (ES⁺). Intermediate L1 + Intermediate R13 |
| 5 | 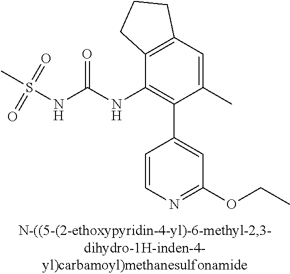 [SMILES: CCOc1cc(-c2c(C)cc3c(c2NC(=O)NS(C)(=O)=O)CCC3)ccn1] | ¹H NMR (DMSO-d6) δ 8.19 (d, J = 5.1 Hz, 1H), 7.47 (s, 1H), 7.10 (s, 1H), 6.75-6.70 (m, 1H), 6.56 (s, 1H), 4.33 (q, J = 7.0 Hz, 2H), 3.04 (s, 3H), 2.90 (t, J = 7.4 Hz, 2H), 2.78-2.70 (m, 2H), 2.05-1.94 (m, 5H), 1.34 (t, J = 7.1 Hz, 3H). One exchangeable proton not observed. LCMS m/z 390.2 (M + H)⁺ (ES⁺). Intermediate L1 + Intermediate R29 |
| 6 | 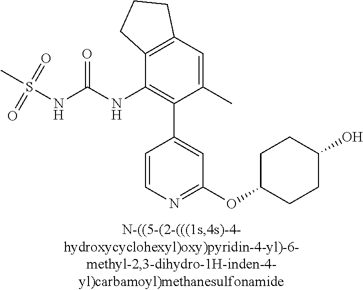 [SMILES: Cc1cc2c(c(NC(=O)NS(C)(=O)=O)c1-c1ccnc(O[C@H]3CC[C@@H](O)CC3)c1)CCC2] | ¹H NMR (DMSO-d6) δ 8.14 (d, J = 5.2 Hz, 1H), 7.14 (s, 1H), 7.05 (s, 1H), 6.74-6.68 (m, 1H), 6.52 (s, 1H), 5.05 (tt, J = 7.1, 3.4 Hz, 1H), 4.47 (d, J = 3.9 Hz, 1H), 3.68- 3.60 (m, 1H), 3.17 (d, J = 3.3 Hz, 1H), 2.92- 2.84 (m, 4H), 2.76 (s, 1H), 2.03-1.94 (m, 5H), 1.92-1.83 (m, 2H), 1.72-1.64 (m, 3H), 1.60 (dd, J = 7.7, 4.1 Hz, 4H). One exchangeable proton not observed. LCMS m/z 460.4 (M + H)⁺ (ES⁺). Intermediate L1 + Intermediate R21 |
| 7 | 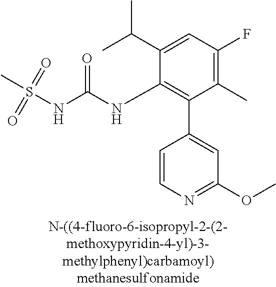 | ¹H NMR (DMSO-d6) δ 8.17 (d, J = 5.2 Hz, 1H), 7.60 (s, 1H), 7.13 (d, J = 11.1 Hz, 1H), 6.79 (s, 1H), 6.64 (s, 1H), 3.87 (s, 3H), 3.16-3.09 (m, 1H), 2.74 (s, 3H), 1.88 (s, 3H), 1.14 (d, J = 6.9 Hz, 6H). One exchangeable proton not observed. LCMS m/z 396.3 (M + H)⁺ (ES⁺). Intermediate L1 + Intermediate R33 |
| 8 | 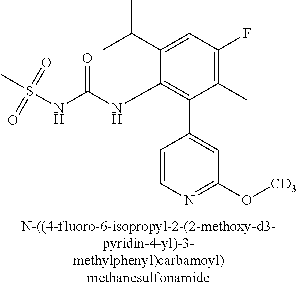 [SMILES: [2H]C([2H])([2H])Oc1cc(-c2c(C)c(F)cc(C(C)C)c2NC(=O)NS(C)(=O)=O)ccn1] | ¹H NMR (DMSO-d6) δ 8.19 (d, J = 5.2 Hz, 1H), 7.40 (s, 1H), 7.16 (d, J = 11.0 Hz, 1H), 6.88-6.74 (m, 1H), 6.64 (s, 1H), 3.16- 3.05 (m, 1H), 2.82 (s, 3H), 1.89 (s, 3H), 1.15 (d, J = 5.2 Hz, 6H). One exchangeable proton not observed. LCMS m/z 399.4 (M + H)⁺ (ES⁺). Intermediate L1 + Intermediate R17 |
| 9 | 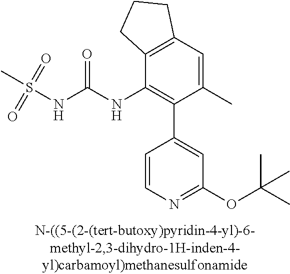 [SMILES: Cc1cc2c(c(NC(=O)NS(C)(=O)=O)c1-c1ccnc(OC(C)(C)C)c1)CCC2] | ¹H NMR (DMSO-d6) δ 8.16 (d, J = 5.2 Hz, 1H), 7.50 (s, 1H), 7.10 (s, 1H), 6.68 (dd, J = 5.2, 1.5 Hz, 1H), 6.43 (s, 1H), 3.04 (s, 3H), 2.90 (t, J = 7.5 Hz, 2H), 2.78-2.71 (m, 2H), 2.05-1.98 (m, 5H), 1.57 (s, 9H). One exchangeable proton not observed. LCMS m/z 362.6 (M-tBu + H)⁺ (ES⁺). Intermediate L1 + Intermediate R30 |
| 10 | 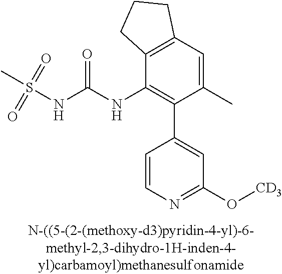 [SMILES: [2H]C([2H])([2H])Oc1cc(-c2c(C)cc3c(c2NC(=O)NS(C)(=O)=O)CCC3)ccn1] | ¹H NMR (DMSO-d6) δ 8.21 (d, J = 5.2 Hz, 1H), 7.50 (s, 1H), 7.11 (s, 1H), 6.75 (d, J = 5.2 Hz, 1H), 6.59 (s, 1H), 3.05 (s, 3H), 2.90 (t, J = 7.4 Hz, 2H), 2.78-2.71 (m, 2H), 2.03-1.97 (m, 5H). One exchangeable proton not observed. LCMS m/z 379.2 (M + H)⁺ (ES⁺). Intermediate L1 + Intermediate R16 |
| 11 | 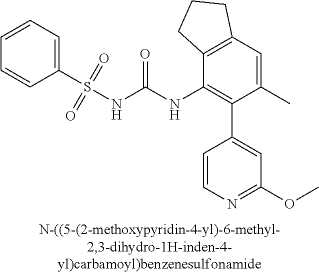 [SMILES: COc1cc(-c2c(C)cc3c(c2NC(=O)NS(=O)(=O)c2ccccc2)CCC3)ccn1] | ¹H NMR (DMSO-d6) δ 10.62 (s, 1H), 8.09 (d, J = 5.2 Hz, 1H), 7.83-7.76 (m, 2H), 7.67 (t, J = 7.4 Hz, 1H), 7.59 (t, J = 7.6 Hz, 2H), 7.46 (s, 1H), 7.06 (s, 1H), 6.54 (d, J = 4.8 Hz, 1H), 6.49 (s, 1H), 3.89 (s, 3H), 2.85 (t, J = 7.4 Hz, 2H), 1.97-1.87 (m, 5H). Two protons under solvent. LCMS m/z 438.6 (M + H)⁺ (ES⁺). Intermediate L5 + Intermediate R25 |
| 12 | 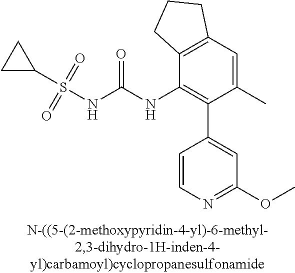 [SMILES: COc1cc(-c2c(C)cc3c(c2NC(=O)NS(=O)(=O)C2CC2)CCC3)ccn1] | ¹H NMR (DMSO-d6) δ 8.22 (d, J = 5.2 Hz, 1H), 7.52 (s, 1H), 7.12 (s, 1H), 6.75 (dd, J = 5.2, 1.4 Hz, 1H), 6.60 (s, 1H), 3.89 (s, 3H), 2.90 (t, J = 7.4 Hz, 2H), 2.80-2.70 (m, 3H), 2.05-1.97 (m, 5H), 1.00-0.93 (m, 4H). One exchangeable proton not observed. LCMS m/z 402.5 (M + H)⁺ (ES⁺). Intermediate L2 + Intermediate R25 |
| 13 | 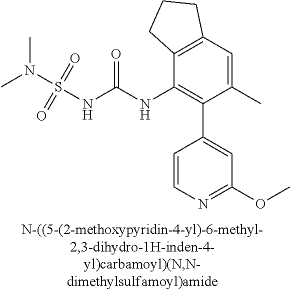 | ¹H NMR (DMSO-d6) δ 9.66 (s, 1H), 8.22 (d, J = 5.2 Hz, 1H), 7.46 (s, 1H), 7.11 (s, 1H), 6.75 (dd, J = 5.3, 1.4 Hz, 1H), 6.59 (s, 1H), 3.89 (s, 3H), 2.90 (t, J = 7.4 Hz, 2H), 2.76-2.70 (m, 2H), 2.66 (s, 6H), 2.05- 1.96 (m, 5H). LCMS m/z 405.2 (M + H)⁺ (ES⁺). Intermediate L3 + Intermediate R25 |
| 14 | 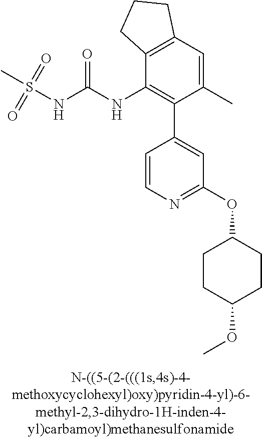 | ¹H NMR (DMSO-d6) δ 8.30-8.04 (m, 1H), 6.97 (d, J = 7.8 Hz, 2H), 6.70 (dd, J = 5.2, 1.4 Hz, 1H), 6.54-6.49 (m, 1H), 5.02- 4.95 (m, 1H), 3.25 (s, 3H), 2.86 (t, J = 7.5 Hz, 2H), 2.80-2.73 (m, 2H), 2.72-2.62 (m, 3H), 2.11-2.03 (m, 2H), 2.02-1.92 (m, 7H), 1.82-1.69 (m, 1H), 1.63 (s, 1H), 1.52-1.43 (m, 2H), 1.39-1.31 (m, 1H). One exchangeable proton not observed. LCMS m/z 474.3 (M + H)⁺ (ES⁺). Intermediate L1 + Intermediate R32 |
| 15 | 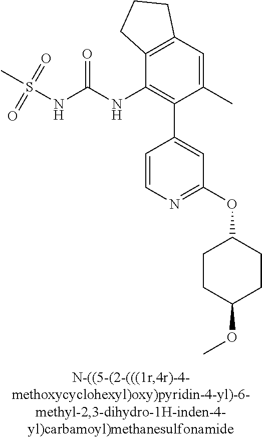 | ¹H NMR (DMSO-d6) δ 8.14 (dd, J = 5.1, 1.8 Hz, 1H), 7.02 (s, 1H), 6.70 (dd, J = 5.2, 1.4 Hz, 1H), 6.52 (d, J = 1.4 Hz, 1H), 5.06 (s, 1H), 3.24 (s, 3H), 2.88 (t, J = 7.4 Hz, 2H), 2.84-2.72 (m, 5H), 2.03-1.94 (m, 5H), 1.83-1.69 (m, 5H), 1.67-1.59 (m, 2H). Two protons masked by water, two exchangeable protons not observed. LCMS m/z 474.4 (M + H)⁺ (ES⁺). Intermediate L1 + Intermediate R32 |
| 16 | 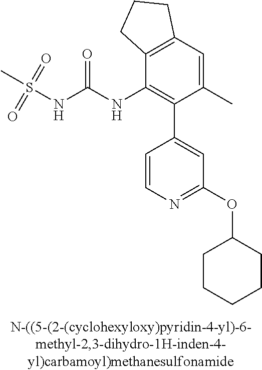 [SMILES: Cc1cc2c(c(NC(=O)NS(C)(=O)=O)c1-c1ccnc(OC3CCCCC3)c1)CCC2] | ¹H NMR (DMSO-d6) δ 8.16 (d, J = 5.2 Hz, 1H), 7.31 (s, 1H), 7.07 (s, 1H), 6.70 (dd, J = 5.2, 1.4 Hz, 1H), 6.50 (s, 1H), 5.05-4.96 (m, 1H), 2.96 (s, 3H), 2.89 (t, J = 7.5 Hz, 2H), 2.79-2.72 (m, 2H), 2.05-1.96 (m, 7H), 1.78-1.71 (m, 2H), 1.60-1.53 (m, 1H), 1.51-1.32 (m, 4H), 1.31-1.20 (m, 1H). One exchangeable proton not observed. LCMS m/z 444.3 (M + H)⁺ (ES⁺). Intermediate L1 + Intermediate R31 |
| 17 | 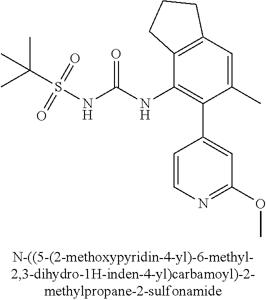 [SMILES: COc1cc(-c2c(C)cc3c(c2NC(=O)NS(=O)(=O)C(C)(C)C)CCC3)ccn1] | ¹H NMR (DMSO-d6) δ 8.20 (d, J = 5.1 Hz, 1H), 7.65 (s, 1H), 7.10 (s, 1H), 6.74 (dd, J = 5.1, 1.3 Hz, 1H), 6.56 (s, 1H), 3.88 (s, 3H), 2.89 (t, J = 7.4 Hz, 2H), 2.75-2.69 (m, 2H), 1.99 (d, J = 4.8 Hz, 5H), 1.18 (s, 9H). One exchangeable proton not observed. LCMS m/z 418.2 (M + H)⁺ (ES⁺). Intermediate L6 + Intermediate R25 |
| 18 | 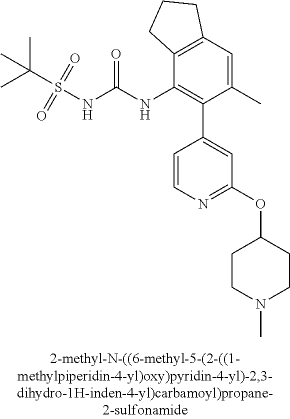 [SMILES: Cc1cc2c(c(NC(=O)NS(=O)(=O)C(C)(C)C)c1-c1ccnc(OC3CCN(C)CC3)c1)CCC2] | ¹H NMR (DMSO-d6) δ 8.16 (d, J = 5.2 Hz, 1H), 7.71 (s, 1H), 7.09 (s, 1H), 6.70 (dd, J = 5.2, 1.4 Hz, 1H), 6.51 (d, J = 1.2 Hz, 1H), 5.01 (tt, J = 8.8, 4.1 Hz, 1H), 2.89 (t, J = 7.5 Hz, 2H), 2.80-2.68 (m, 4H), 2.36- 2.20 (m, 5H), 2.07-1.88 (m, 7H), 1.76- 1.65 (m, 2H), 1.17 (s, 9H). One exchangeable proton not observed. LCMS m/z 501.3 (M + H)⁺ (ES⁺). Intermediate L6 + Intermediate R26 |
| 19 | 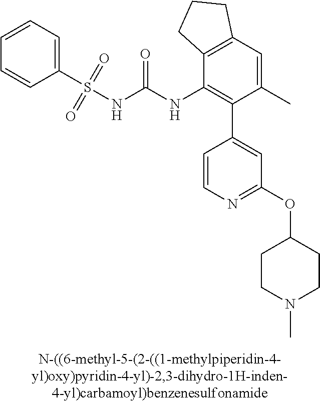 [SMILES: Cc1cc2c(c(NC(=O)NS(=O)(=O)c3ccccc3)c1-c1ccnc(OC3CCN(C)CC3)c1)CCC2] | ¹H NMR (DMSO-d6) δ 8.10 (d, J = 5.2 Hz, 1H), 7.74-7.69 (m, 2H), 7.55-7.50 (m, 1H), 7.48 (dd, J = 8.2, 6.5 Hz, 2H), 7.26- 7.17 (m, 1H), 7.00 (s, 1H), 6.61 (dd, J = 5.2, 1.4 Hz, 1H), 6.51 (s, 1H), 5.13-5.09 (m, 1H), 3.08-3.00 (m, 2H), 2.83 (t, J = 7.5 Hz, 2H), 2.78-2.67 (m, 2H), 2.14- 2.04 (m, 2H), 1.96 (s, 3H), 1.94-1.87 (m, 2H), 1.87-1.80 (m, 2H). Five protons under DMSO. One exchangeable proton not observed. LCMS m/z 521.2 (M + H)⁺ (ES⁺). Intermediate L5 + Intermediate R26 |
| 20 | 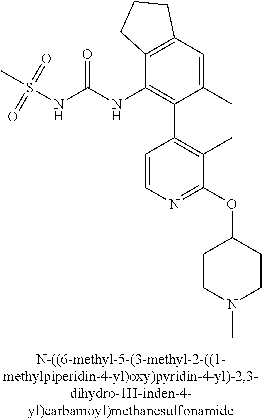 [SMILES: Cc1cc2c(c(NC(=O)NS(C)(=O)=O)c1-c1ccnc(OC3CCN(C)CC3)c1C)CCC2] | ¹H NMR (DMSO-d6) δ 8.00 (d, J = 5.0 Hz, 1H), 7.07 (s, 2H), 6.59 (d, J = 5.2 Hz, 1H), 5.15-5.06 (m, 1H), 2.95-2.88 (m, 5H), 2.82-2.68 (m, 4H), 2.45-2.37 (m, 2H), 2.30 (s, 3H), 2.05-1.96 (m, 4H), 1.88 (s, 3H), 1.80-1.71 (m, 5H). One exchangeable proton not observed. LCMS m/z 473.4 (M + H)⁺ (ES⁺). Intermediate L1 + Intermediate R23 |
| 21 | 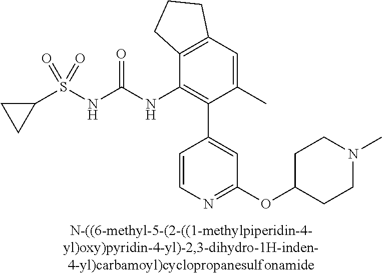 [SMILES: Cc1cc2c(c(NC(=O)NS(=O)(=O)C3CC3)c1-c1ccnc(OC3CCN(C)CC3)c1)CCC2] | ¹H NMR (DMSO-d6) δ 9.94 (s, 1H), 8.18 (d, J = 5.2 Hz, 1H), 7.46 (s, 1H), 7.09 (s, 1H), 6.73 (dd, J = 5.2, 1.4 Hz, 1H), 6.54 (s, 1H), 5.02 (tt, J = 8.7, 4.1 Hz, 1H), 2.90 (t, J = 7.4 Hz, 2H), 2.83-2.69 (m, 5H), 2.37- 2.24 (m, 5H), 2.07-1.97 (m, 7H), 1.77- 1.66 (m, 2H), 0.97-0.86 (m, 4H). LCMS m/z 485.1 (M + H)⁺ (ES⁺). Intermediate L2 + Intermediate R26 |
| 22 | 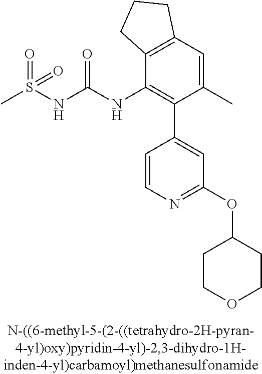 [SMILES: Cc1cc2c(c(NC(=O)NS(C)(=O)=O)c1-c1ccnc(OC3CCOCC3)c1)CCC2] | ¹H NMR (DMSO-d6) δ 8.18 (d, J = 5.2 Hz, 1H), 7.38 (s, 1H), 7.09 (s, 1H), 6.73 (dd, J = 5.2, 1.4 Hz, 1H), 6.56 (d, J = 1.4 Hz, 1H), 5.25-5.16 (m, 1H), 3.93-3.83 (m, 2H), 3.54-3.46 (m, 2H), 2.97 (s, 3H), 2.90 (t, J = 7.4 Hz, 2H), 2.81-2.68 (m, 2H), 2.09- 1.96 (m, 7H), 1.72-1.59 (m, 2H). One exchangeable proton not observed. LCMS m/z 446.2 (M + H)⁺ (ES⁺); m/z 444.2 (M − H)⁻ (ES⁻). Intermediate L1 + Intermediate R20 |
| 23 | 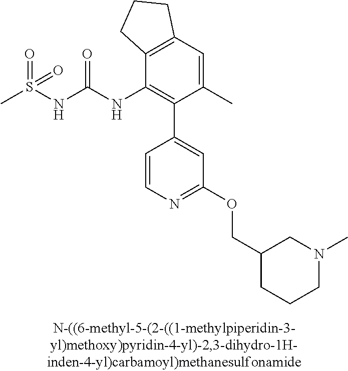 | ¹H NMR (DMSO-d6) δ 8.17 (d, J = 5.1 Hz, 1H), 7.25 (s, 1H), 7.07 (s, 1H), 6.75 (d, J = 5.2 Hz, 1H), 6.59 (s, 1H), 4.33-4.01 (m, 2H), 3.08-2.97 (m, 1H), 2.95-2.82 (m, 6H), 2.76 (t, J = 7.6 Hz, 2H), 2.42-2.30 (m, 3H), 2.26-2.04 (1H, 2H), 2.04-1.93 (m, 6H), 1.78-1.66 (m, 2H), 1.63-1.50 (m, 1H), 1.16-1.04 (m, 1H). One exchangeable proton not observed. LCMS m/z 473.0 (M + H)⁺ (ES⁺); m/z 471.2 (M − H)⁻ (ES⁻). Intermediate L1 + Intermediate R22 |
| 24 | 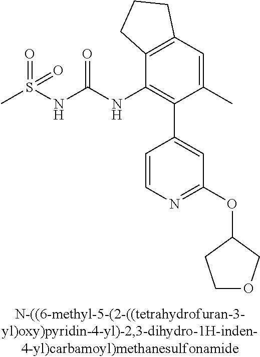 [SMILES: Cc1cc2c(c(NC(=O)NS(C)(=O)=O)c1-c1ccnc(OC3CCOC3)c1)CCC2] | ¹H NMR (DMSO-d6) δ 8.18 (d, J = 5.2 Hz, 1H), 7.37 (s, 1H), 7.08 (s, 1H), 6.75 (dd, J = 5.2, 1.4 Hz, 1H), 6.58 (s, 1H), 5.56-5.49 (m, 1H), 3.96-3.89 (m, 1H), 3.89-3.83 (m, 1H), 3.83-3.73 (m, 2H), 2.96 (s, 3H), 2.90 (t, J = 7.4 Hz, 2H), 2.80-2.70 (m, 2H), 2.29-2.20 (m, 1H), 2.07-1.95 (m, 6H). One exchangeable proton not observed. LCMS m/z 432.2 (M + H)⁺ (ES⁺); m/z 430.1 (M − H)⁻ (ES⁻). Intermediate L1 + Intermediate R18 |
| 25 | 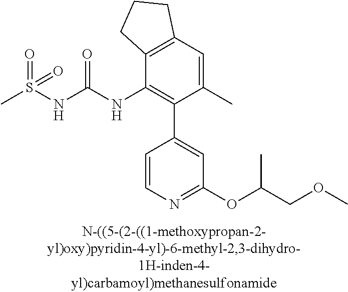 | ¹H NMR (DMSO-d6) δ 10.11 (s, 1H), 8.19 (d, J = 5.2 Hz, 1H), 7.55 (s, 1H), 7.12 (s, 1H), 6.72 (d, J = 5.3 Hz, 1H), 6.52 (s, 1H), 5.38-5.31 (m, 1H), 3.58-3.51 (m, 1H), 3.49-3.44 (m, 1H), 3.29 (s, 3H), 3.07 (s, 3H), 2.91 (t, J = 7.4 Hz, 2H), 2.78-2.70 (m, 2H), 2.05-1.97 (m, 5H), 1.27 (d, J = 6.4 Hz, 3H). LCMS m/z 434.2 (M + H)⁺ (ES⁺); m/z 432.2 (M − H)⁻ (ES⁻). Intermediate L1 + Intermediate R19 |
| 26 | 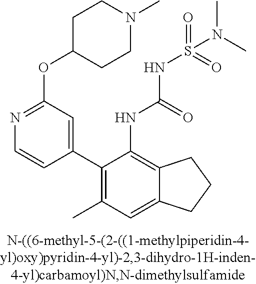 [SMILES: Cc1cc2c(c(NC(=O)NS(=O)(=O)N(C)C)c1-c1ccnc(OC3CCN(C)CC3)c1)CCC2] | ¹H NMR (DMSO-d₆) δ 8.19 (d, J = 5.2 Hz, 1H), 7.46 (bs, 1H), 7.10 (s, 1H), 6.72 (dd, J = 5.2, 1.4 Hz, 1H), 6.52 (s, 1H), 5.04-4.95 (m, 1H), 2.90 (t, J = 7.4 Hz, 2H), 2.77- 2.69 (m, 4H), 2.64 (s, 6H), 2.28-2.16 (m, 5H), 2.09-1.94 (m, 7H), 1.73-1.64 (m, 2H). One exchangeable proton not observed. LCMS m/z 488.4 (M + H)⁺ (ES⁺); m/z 486.3 (M − H)⁻ (ES⁻). Intermediate L3 + Intermediate R26 |
| 27 | 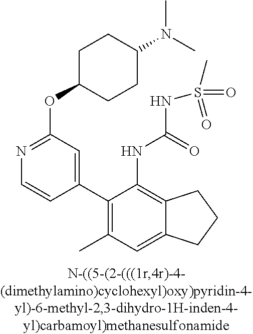 | ¹H NMR (DMSO-d₆) δ 8.16 (d, J = 5.2 Hz, 1H), 7.17 (bs, 1H), 7.04 (s, 1H), 6.71 (dd, J = 5.2, 1.4 Hz, 1H), 6.51 (s, 1H), 4.97-4.88 (m, 1H), 2.95-2.82 (m, 5H), 2.79-2.72 (m, 2H), 2.66-2.59 (m, 1H), 2.42 (s, 6H), 2.21- 2.15 (m, 2H), 2.04-1.87 (m, 7H), 1.53-1.38 (m, 4H). One exchangeable proton not observed. LCMS m/z 487.4 (M + H)⁺ (ES⁺); m/z 485.3 (M − H)⁻ (ES⁻). Intermediate L1 + Intermediate R24 |
| 28 | 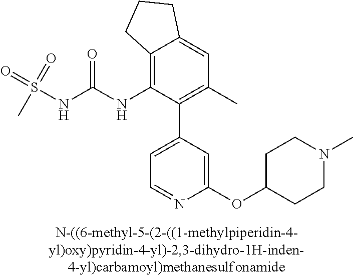 [SMILES: Cc1cc2c(c(NC(=O)NS(C)(=O)=O)c1-c1ccnc(OC3CCN(C)CC3)c1)CCC2] | ¹H NMR (DMSO-d6) δ 8.17 (t, J = 5.7 Hz, 1H), 7.35 (s, 1H), 7.08 (d, J = 5.1 Hz, 1H), 6.74-6.71 (m, 1H), 6.55-6.54 (m, 1H), 5.06-5.00 (m, 1H), 3.00-2.94 (m, 3H), 2.92-2.87 (m, 2H), 2.84-2.78 (m, 2H), 2.76-2.72 (m, 2H), 2.42-2.35 (m, 2H) 2.34-2.28 (m, 3H), 2.08-1.93 (m, 7H), 1.78-1.64 (m, 2H). One exchangeable proton not observed. LCMS m/z 459.4 (M + H)⁺ (ES⁺). Intermediate L1 + Intermediate R26 |
| 29 | 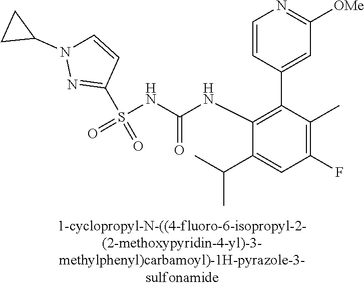 [SMILES: COc1cc(-c2c(C)c(F)cc(C(C)C)c2NC(=O)NS(=O)(=O)c2ccn(C3CC3)n2)ccn1] | ¹H NMR (DMSO-d6) δ 8.10 (s, 1H), 7.92 (s, 1H), 7.48 (s, 1H), 7.14 (d, J = 11.0 Hz, 1H), 6.81-6.35 (m, 3H), 3.89 (s, 3H), 3.87-3.83 (m, 1H), 2.95-2.84 (m, 1H), 1.85 (s, 3H), 1.10-1.03 (m, 10H). One exchangeable proton not observed. LCMS m/z 488.3 (M + H)⁺ (ES⁺). Intermediate R33 + known sulfonamide |
| 30 | 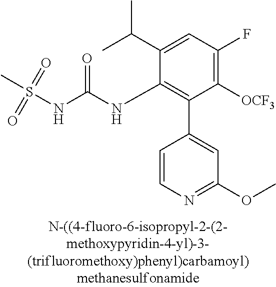 | ¹H NMR (DMSO-d6) δ 8.23 (d, J = 5.3 Hz, 1H), 7.82 (s, 1H), 7.58 (d, J = 11.7 Hz, 1H), 6.89 (s, 1H), 6.75 (s, 1H), 3.88 (s, 3H), 3.21-3.10 (m, 1H), 2.90 (s, 3H), 1.18 (d, J = 6.9 Hz, 6H). One exchangeable proton not observed. LCMS m/z 466.1 (M + H)⁺ (ES⁺). Intermediate L1 + Intermediate R37 |
| 31 | 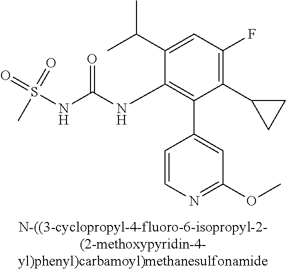 [SMILES: COc1cc(-c2c(NC(=O)NS(C)(=O)=O)c(C(C)C)cc(F)c2C2CC2)ccn1] | ¹H NMR (DMSO-d6) δ 8.19 (d, J = 5.2 Hz, 1H), 7.47 (s, 1H), 7.14 (d, J = 12.2 Hz, 1H), 6.85 (s, 1H), 6.69 (s, 1H), 3.88 (s, 3H), 3.11-3.01 (m, 1H), 2.93 (s, 3H), 1.56-1.47 (m, 1H), 1.18-1.09 (m, 6H), 0.62-0.54 (m, 2H), 0.50-0.41 (m, 2H). One exchangeable proton not observed. LCMS m/z 422.2 (M + H)⁺ (ES⁺). Intermediate L1 + Intermediate R35 |
| 32 | 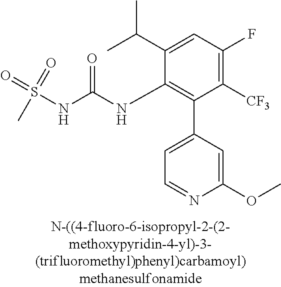 [SMILES: COc1cc(-c2c(NC(=O)NS(C)(=O)=O)c(C(C)C)cc(F)c2C(F)(F)F)ccn1] | ¹H NMR (DMSO-d6) δ 8.19 (d, J = 5.1 Hz, 1H), 7.58 (d, J = 13.2 Hz, 2H), 6.81 (s, 1H), 6.64 (s, 1H), 3.88 (s, 3H), 3.19-3.07 (m, 1H), 2.90 (s, 3H), 1.24-1.11 (m, 6H). One exchangeable proton not observed. LCMS m/z 450.3 (M + H)⁺ (ES⁺). Intermediate L1 + Intermediate R36 |
| 33 | 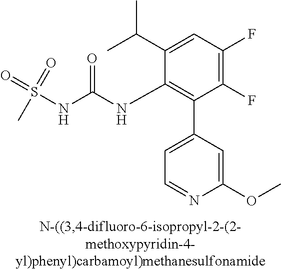 [SMILES: COc1cc(-c2c(F)c(F)cc(C(C)C)c2NC(=O)NS(C)(=O)=O)ccn1] | ¹H NMR (DMSO-d6) δ 8.23 (d, J = 5.2 Hz, 1H), 7.84 (s, 1H), 7.48 (t, J = 10.4 Hz, 1H), 6.99-6.89 (m, 1H), 6.81 (s, 1H), 3.88 (s, 3H), 3.18-3.06 (m, 1H), 2.90 (s, 3H), 1.16 (d, J = 6.8 Hz, 6H). One exchangeable proton not observed. LCMS m/z 400.3 (M + H)⁺ (ES⁺). Intermediate L1 + Intermediate R34 |
| 34 | 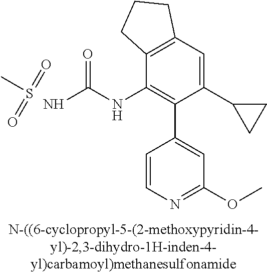 | ¹H NMR (DMSO-d6) δ 8.15 (d, J = 5.1 Hz, 1H), 6.83-6.79 (m, 1H), 6.67-6.62 (m, 2H), 6.54 (s, 1H), 3.87 (s, 3H), 2.84 (t, J = 7.4 Hz, 2H), 2.80-2.75 (m, 2H), 1.98- 1.91 (m, 2H), 1.48-1.41 (m, 1H), 0.68- 0.63 (m, 2H), 0.57-0.52 (m, 2H). One exchangeable proton not observed. Three protons under DMSO. LCMS m/z 402.3 (M + H)⁺ (ES⁺). Intermediate L1 + Intermediate R14 |
| 35 | 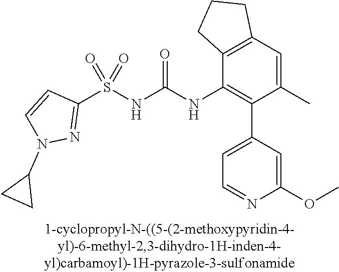 [SMILES: COc1cc(-c2c(C)cc3c(c2NC(=O)NS(=O)(=O)c2ccn(C4CC4)n2)CCC3)ccn1] | ¹H NMR (DMSO-d6) δ 10.67 (s, 1H), 8.16 (d, J = 5.2 Hz, 1H), 7.96 (d, J = 2.4 Hz, 1H), 7.47 (s, 1H), 7.08 (s, 1H), 6.65 (d, J = 5.2 Hz, 1H), 6.61-6.47 (m, 2H), 3.93- 3.83 (m, 4H), 2.87 (t, J = 7.5 Hz, 2H), 2.61- 2.52 (m, 2H), 2.02-1.88 (m, 5H), 1.11- 0.98 (M, 4H). LCMS m/z 468.3 (M + H)⁺ (ES⁺). Intermediate R25 + known sulfonamide |
| 36 | 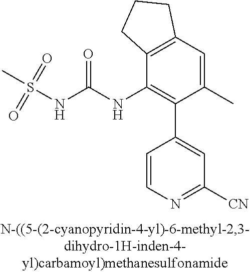 [SMILES: Cc1cc2c(c(NC(=O)NS(C)(=O)=O)c1-c1ccnc(C#N)c1)CCC2] | ¹H NMR (DMSO-d6) δ 8.80-8.76 (m, 1H), 7.85 (s, 1H), 7.72 (s, 1H), 7.53 (dd, J = 5.0, 1.7 Hz, 1H), 7.15 (s, 1H), 2.99-2.89 (m, 5H), 2.77 (t, J = 7.7 Hz, 2H), 2.04- 1.98 (m, 5H). Acidic NH not observed. LCMS m/z 371.1 (M + H)⁺ (ES⁺). Intermediate L1 + Intermediate R10 |
| 37 | 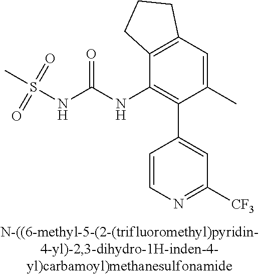 [SMILES: Cc1cc2c(c(NC(=O)NS(C)(=O)=O)c1-c1ccnc(C(F)(F)F)c1)CCC2] | ¹H NMR (DMSO-d6) δ 8.81 (d, J = 4.9 Hz, 1H), 7.77-7.68 (m, 1H), 7.67 (s, 1H), 7.52 (dd, J = 4.9, 1.5 Hz, 1H), 7.16 (s, 1H), 2.97- 2.88 (m, 5H), 2.78 (t, J = 7.4 Hz, 2H), 2.07-1.98 (m, 5H). Acidic NH not observed. LCMS m/z 414.1 (M + H)⁺ (ES⁺). Intermediate L1 + Intermediate R11 |
| 38 | 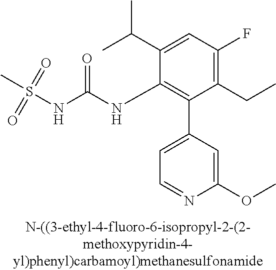 [SMILES: CCc1c(F)cc(C(C)C)c(NC(=O)NS(C)(=O)=O)c1-c1ccnc(OC)c1] | ¹H NMR (DMSO-d6) δ 10.06 (s, 1H), 8.21 (d, J = 5.2 Hz, 1H), 7.52 (s, 1H), 7.21 (d, J = 11.4 Hz, 1H), 6.80 (s, 1H), 6.64 (s, 1H), 3.88 (s, 3H), 3.10-3.00 (m, 1H), 2.96 (s, 3H), 2.33-2.25 (m, 2H), 1.24-1.09 (m, 6H), 0.94 (t, J = 7.5 Hz, 3H). LCMS m/z 410.2 (M + H)⁺ (ES). Intermediate L1 + Intermediate R38 |
| 39 | 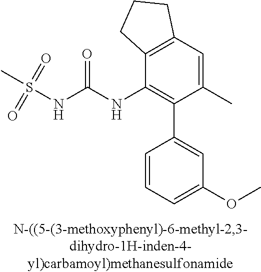 | ¹H NMR (DMSO-d6) δ 7.36 (t, J = 7.8 Hz, 1H), 7.30 (s, 1H), 7.07 (s, 1H), 6.97-6.89 (m, 1H), 6.73-6.63 (m, 2H), 3.76 (s, 3H), 3.04 (s, 3H), 2.89 (t, J = 7.5 Hz, 2H), 2.75 (t, J = 7.6 Hz, 2H), 2.05-1.93 (m, 5H). Acidic NH not observed. LCMS m/z 375.2 (M + H)⁺ (ES⁺). Intermediate L1 + Intermediate R12 |

### Example 40

### N-((5-(2-Methoxypyridin-4-yl)-6-methyl-2,3-dihydro-1H-inden-4-yl)carbamoyl)methanesulfonamide, sodium salt

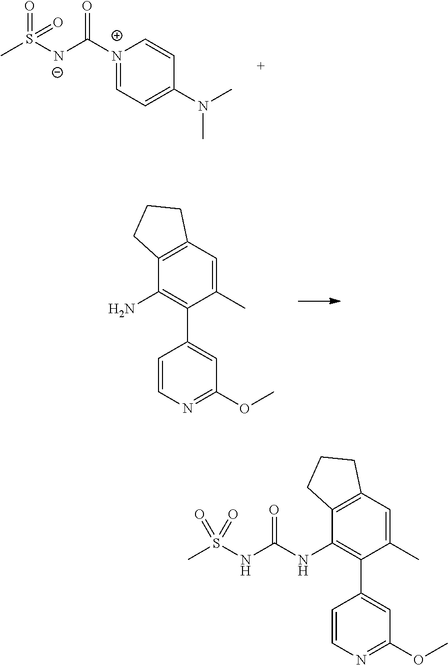

5-(2-Methoxypyridin-4-yl)-6-methyl-2,3-dihydro-1H-inden-4-amine (Intermediate R25) (148 mg, 0.582 mmol) was added to a suspension of (4-(dimethylamino)pyridin-1-ium-1-carbonyl)(methylsulfonyl)amide (Intermediate L1) (142 mg, 0.582 mmol) in MeCN (2 mL). The reaction mixture was stirred at 60° C. for 1 hour. Volatiles were evaporated, and the crude product was dissolved in DMSO (2 mL) and filtered. The crude product was purified by basic prep HPLC to afford the free acid, which was treated with 0.5 M NaOH (474 μL, 0.237 mmol) and freeze dried to afford the title compound (83 mg, 87%) as a white solid.

¹H NMR (DMSO-d6) δ 8.15 (dd, J=5.2, 0.7 Hz, 1H), 6.95 (s, 1H), 6.75 (dd, J=5.2, 1.4 Hz, 1H), 6.63-6.54 (m, ²H), 3.87 (s, 3H), 2.86 (t, J=7.4 Hz, 2H), 2.79 (t, J=7.5 Hz, 2H), 2.58 (s, 3H), 2.04-1.89 (m, 5H).

LCMS m/z 376.2 (M+H)⁺(ES⁺).

### Example 41

### 1-(2-(Dimethylamino)ethyl)-N-((6-methyl-5-(2-((1-methylpiperidin-4-yl)oxy)pyridin-4-yl)-2,3-dihydro-1H-inden-4-yl)carbamoyl)-1H-pyrazole-3-sulfonamide

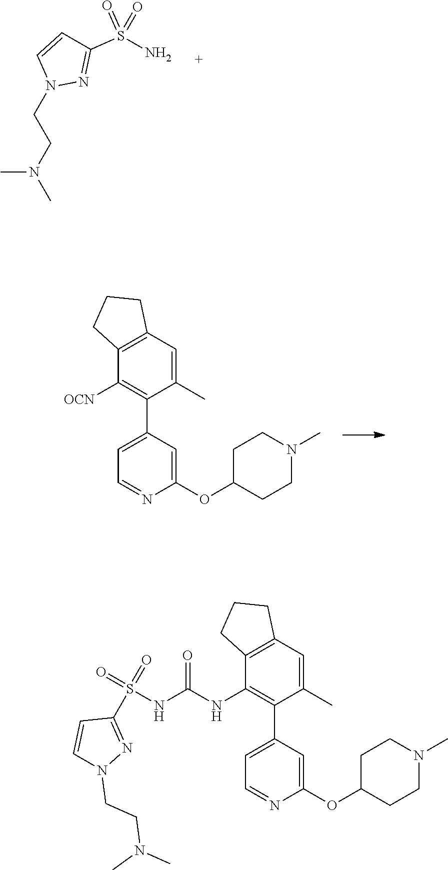

To a stirred solution of 1-(2-(dimethylamino)ethyl)-1H-pyrazole-3-sulfonamide (22 mg, 0.1 mmol) in THF (2 mL) at room temperature was added 2M sodium tert-butoxide in THF (0.055 mL, 0.11 mmol). The resulting mixture was stirred at room temperature for 1 hour. Then a solution of 4-(4-isocyanato-6-methyl-2,3-dihydro-1H-inden-5-yl)-2-((1-methylpiperidin-4-yl)oxy)pyridine (Intermediate R27) (36.3 mg, 0.1 mmol) in THF (1 mL) was added and the reaction mixture stirred at room temperature overnight. Volatiles were evaporated, and the crude product dissolved in DMSO (1 mL) and purified by basic prep HPLC (10-40% MeCN in water) to afford the title compound (5 mg, 9%) as a solid.

¹H NMR (DMSO-d6) δ 8.13 (d, J=5.3 Hz, 1H), 7.79 (s, 1H), 7.25 (s, 1H), 7.02 (s, 1H), 6.67 (d, J=5.3 Hz, 1H), 6.53 (s, 1H), 6.47-6.40 (m, 1H), 5.13-5.04 (m, 1H), 4.23 (t, J=6.6 Hz, 2H), 2.96-2.89 (m, 2H), 2.85 (t, J=7.4 Hz, 2H), 2.66 (t, J=6.6 Hz, 2H), 2.62-2.56 (m, 4H), 2.41 (s, 3H), 2.19 (s, 6H), 2.10-2.03 (m, 2H), 1.98 (s, 3H), 1.96-1.90 (m, 2H), 1.87-1.73 (m, 2H). One exchangeable proton not observed.

LCMS m/z 582.3 (M+H)⁺(ES⁺).

The following examples were prepared according to the general procedure of Example 41:

| Ex. | Structure and name | Characterisation and procedure |
|---|---|---|
| 42 | 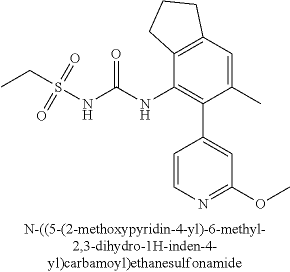 [SMILES: CCS(=O)(=O)NC(=O)Nc1c2c(cc(C)c1-c1ccnc(OC)c1)CCC2] | ¹H NMR (DMSO-d6) δ 8.20 (d, J = 5.2 Hz, 1H), 7.48 (s, 1H), 7.10 (s, 1H), 6.75 (d, J = 5.2 Hz, 1H), 6.59 (s, 1H), 3.88 (s, 3H), 3.15 (q, J = 7.3 Hz, 2H), 2.90 (t, J = 7.4 Hz, 2H), 2.79 -2.69 (m, 2H), 2.05-1.94 (m, 5H), 1.08 (t, J = 7.3 Hz, 3H). One exchangeable proton not observed. LCMS m/z 390.2 (M + H)⁺ (ES⁺). Intermediate L4 + Intermediate R28 |
| 43 | 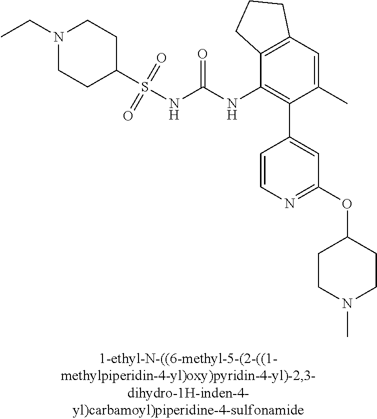 [SMILES: CCN1CCC(S(=O)(=O)NC(=O)Nc2c3c(cc(C)c2-c2ccnc(OC4CCN(C)CC4)c2)CCC3)CC1] | ¹H NMR (DMSO-d6) δ 8.15 (d, J = 5.1 Hz, 1H), 7.37-7.22 (m, 1H), 7.06 (s, 1H), 6.74- 6.67 (m, 1H), 6.52 (s, 1H), 5.07-4.95 (m, 1H), 3.22-3.17 (m, 1H), 3.08-2.98 (m, 2H), 2.89 (t, J = 7.5 Hz, 2H), 2.82-2.70 (m, 4H), 2.26 (s, 5H), 2.07-1.94 (m, 9H), 1.85-1.66 (m, 5H), 1.62-1.52 (m, 2H), 1.04 (t, J = 7.1 Hz, 3H). One exchangeable proton not observed, one proton under DMSO. LCMS m/z 556.3 (M + H)⁺ (ES⁺). Intermediate R27 + known sulfonamide |
| 44 | 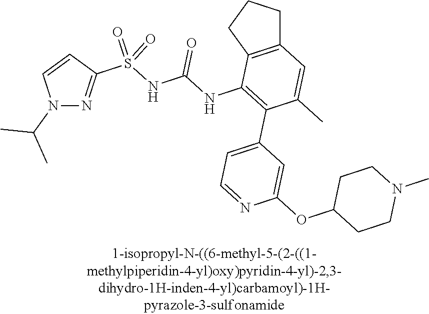 [SMILES: Cc1cc2c(c(NC(=O)NS(=O)(=O)c3ccn(C(C)C)n3)c1-c1ccnc(OC3CCN(C)CC3)c1)CCC2] | ¹H NMR (DMSO-d6) δ 8.16-8.10 (m, 1H), 7.86-7.80 (m, 1H), 7.30 (s, 1H), 7.02 (s, 1H), 6.67 (dd, J = 5.2, 1.6 Hz, 1H), 6.53 (s, 1H), 6.46 (d, J = 2.3 Hz, 1H), 5.11-5.04 (m, 1H), 4.59-4.50 (m, 1H), 3.00-2.89 (m, 2H), 2.85 (t, J = 7.5 Hz, 2H), 2.59- 2.53 (m, 4H), 2.42 (s, 3H), 2.11-2.03 (m, 2H), 1.97 (s, 3H), 1.91 (p, J = 7.7 Hz, 2H), 1.86-1.75 (m, 2H), 1.42 (d, J = 6.6 Hz, 6H). One exchangeable proton not observed. LCMS m/z 553.3 (M + H)⁺ (ES). Intermediate R27 + known sulfonamide |
| 45 | 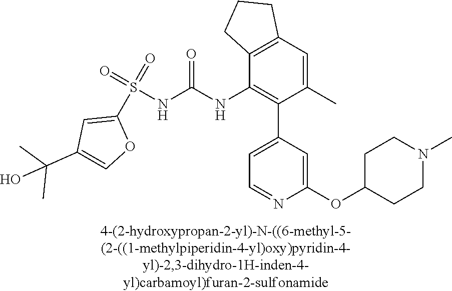 [SMILES: Cc1cc2c(c(NC(=O)NS(=O)(=O)c3cc(C(C)(C)O)co3)c1-c1ccnc(OC3CCN(C)CC3)c1)CCC2] | ¹H NMR (DMSO-d6) δ 9.69 (bs, 1H), 8.15 (d, J = 5.2 Hz, 1H), 7.44 (s, 1H), 7.14- 6.99 (m, 1H), 6.98 (s, 1H), 6.72 (d, J = 5.1 Hz, 1H), 6.67-6.55 (m, 2H), 5.22-5.12 (m, 1H), 4.95 (s, 1H), 3.22 (s, 2H), 3.11 2.94 (m, 2H), 2.85 (t, J = 7.5 Hz, 2H), 2.72- 2.61 (m, 5H), 2.17-2.07 (m, 2H), 2.00- 1.88 (m, 7H), 1.37 (s, 6H). LCMS m/z 569.3 (M + H)⁺ (ES). Intermediate R27 + known sulfonamide |

### Example 46

### 4-(2-Hydroxypropan-2-yl)-N-((4-isopropyl-2-methyl-1-(pyridin-4-yl)-1H-imidazol-5-yl)carbamoyl)furan-2-sulfonamide, ammonium salt

### Step A: ((4-(2-Hydroxypropan-2-yl)furan-2-yl)sulfonyl)(4-isopropylpyridin-1-ium-1-carbonyl)amide

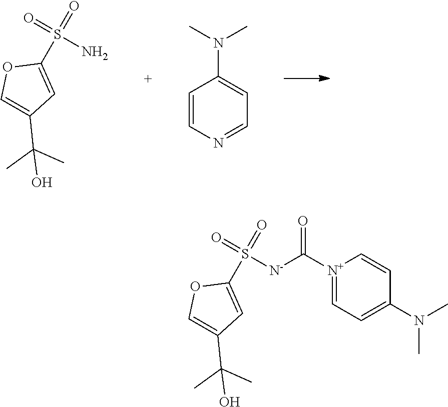

A solution of 4-(2-hydroxypropan-2-yl)furan-2-sulfonamide (Intermediate L₇) (100 mg, 487.26 μmol, 1 eq) and N,N-dimethylpyridin-4-amine (119 mg, 974.51 μmol, 2 eq) in MeCN (2 mL) was stirred at 25° C. for 30 minutes. Then diphenyl carbonate (115 mg, 535.98 μmol, 1.1 eq) was added. The reaction mixture was stirred at 25° C. for 12 hours.

The reaction mixture, a red solution (theoretical amount: 172.19 mg, in 2 mL MeCN), was used directly in the next step.

### Step B

### 4-(2-Hydroxypropan-2-yl)-N-((4-isopropyl-2-methyl-1-(pyridin-4-yl)-1H-imidazol-5-yl)carbamoyl)furan-2-sulfonamide, ammonium salt

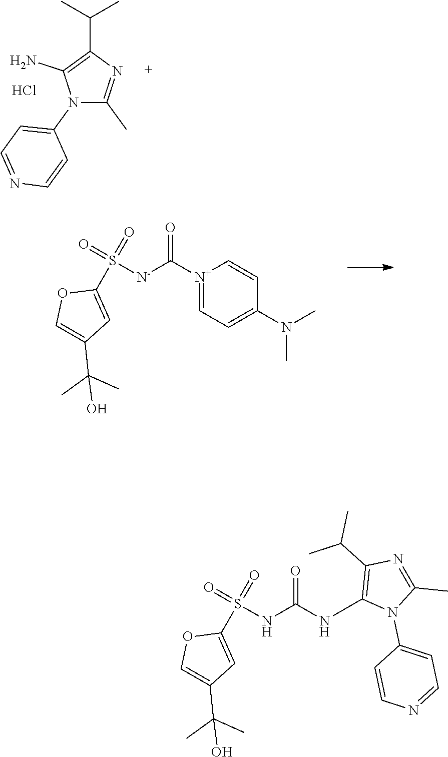 [SMILES: Cc1nc(C(C)C)c(N)n1-c1ccncc1 ; Cl ; CN(C)c1cc[n+](C(=O)[N-]S(=O)(=O)c2cc(C(C)(C)O)co2)cc1 ; Cc1nc(C(C)C)c(NC(=O)NS(=O)(=O)c2cc(C(C)(C)O)co2)n1-c1ccncc1]

To a solution of 4-isopropyl-2-methyl-1-(pyridin-4-yl)-1H-imidazol-5-amine (Intermediate R39) (100 mg, 395.66 μmol, 1 eq, HCl salt) in MeCN (1 mL) was added a solution of ((4-(2-hydroxypropan-2-yl)furan-2-yl)sulfonyl)(4-isopropylpyridin-1-ium-1-carbonyl)amide in MeCN (theoretical amount: 140 mg, 1.6 mL, 395.66 μmol, 1 eq). The reaction mixture was stirred at 70° C. for 1 hour. Then the reaction mixture was purified by reversed phase prep HPLC (column: Waters XBridge C18, 150 mm×25 mm×5 μm; mobile phase [A: water (0.05% ammonium hydroxide v/v); B: MeCN]; B %: 1%-20%, 10 min) to give the title compound (9.52 mg, 5% yield over two steps, 99% purity on LCMS, ammonium salt) as a white solid.

¹H NMR (400 MHz, DMSO-d₆+D₂O) δ 8.71 (s, 2H), 7.51-7.40 (m, 3H), 6.72-6.65 (s, 1H), 2.85-2.81 (m, 1H), 2.33 (s, 3H), 1.37 (s, 6H) and 1.17 (s, 6H).

LCMS: m/z 448.1 (M+H)⁺(ES⁺).

### Example 47

### 1-Cyclopropyl-N-((4-isopropyl-2-methyl-1-(pyridin-4-yl)-1H-imidazol-5-yl)carbamoyl)-1H-pyrazole-3-sulfonamide

### Step A: ((1-Cyclopropyl-1H-pyrazol-3-yl)sulfonyl)(4-(dimethylamino)pyridin-1-ium-1-carbonyl)amide

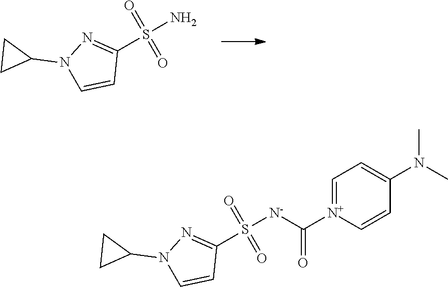 [SMILES: NS(=O)(=O)c1ccn(C2CC2)n1 ; CN(C)c1cc[n+](C(=O)[N-]S(=O)(=O)c2ccn(C3CC3)n2)cc1]

A mixture of 1-cyclopropyl-1H-pyrazole-3-sulfonamide (Intermediate L8) (150 mg, 801.20 μmol, 1 eq) and N,N-dimethylpyridin-4-amine (196 mg, 1.60 mmol, 2 eq) in MeCN (3 mL) was stirred at 25° C. for 30 minutes. Then diphenyl carbonate (189 mg, 881.32 μmol, 1.1 eq) was added. The resulting mixture was stirred at 25° C. for 12 hours. The reaction mixture became turbid and some solid precipitated out. The suspension io was filtered, and the filter cake was collected to give the title compound (95 mg, 35%) as an off-white solid.

¹H NMR (DMSO-d₆) δ 8.10 (d, 2H), 7.92 (d, ¹H), 6.59-6.56 (m, 3H), 3.84-3.75 (m, 1H), 2.95 (s, 6H) and 1.07-0.99 (m, 4H).

### Step B

### 1-Cyclopropyl-N-((4-isopropyl-2-methyl-1-(pyridin-4-yl)-1H-imidazol-5-yl)carbamoyl)-1H-pyrazole-3-sulfonamide

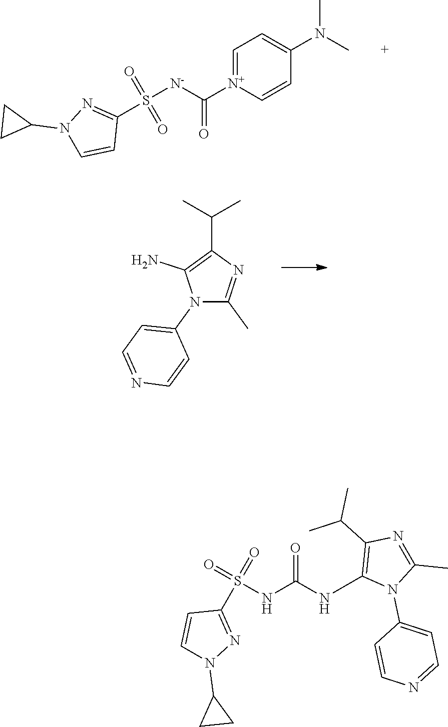 [SMILES: CN(C)c1cc[n+](C(=O)[N-]S(=O)(=O)c2ccn(C3CC3)n2)cc1 ; Cc1nc(C(C)C)c(N)n1-c1ccncc1 ; Cc1nc(C(C)C)c(NC(=O)NS(=O)(=O)c2ccn(C3CC3)n2)n1-c1ccncc1]

To a solution of 4-isopropyl-2-methyl-1-(pyridin-4-yl)-1H-imidazol-5-amine (Intermediate R39) (107 mg, 424.89 μmol, 1.5 eq, HCl salt) in MeCN (4 mL) was added ((1-cyclopropyl-1H-pyrazol-3-yl)sulfonyl)(4-(dimethylamino)pyridin-1-ium-1-carbonyl)amide (95 mg, 283.26 μmol, 1 eq). The reaction mixture was stirred at 70° C. for 45 minutes under N₂. Then the reaction mixture was concentrated in vacuo. The residue was purified by reversed phase flash chromatography (0.1% NH₃.H₂O-MeCN) and then purified by reversed phase prep HPLC (column: Waters XBridge, 150 mm×25 mm×5 μm; mobile phase [A: water (0.05% ammonium hydroxide v/v); B: MeCN]; B %: 1%-20%, 10 min) to give the title compound (11.61 mg, 9% yield over two steps, 98% purity on HPLC) as a white solid.

¹H NMR (400 MHz, DMSO-d₆) δ 8.65-8.62 (m, 2H), 7.89 (s, 1H), 7.79 (d, 1H), 7.34-7.31(m, ²H), 6.47 (s, 1H), 3.83-3.81 (m, 1H), 2.67-2.64 (m, 1H), 2.20 (s, 3H) and 1.10-1.02 (m, 10H).

LCMS: m/z 430.1 (M+H)⁺(ES⁺).

### Example 48

### 4-(2-Hydroxypropan-2-yl)-N-((4-isopropyl-1-(2-methoxypyridin-4-yl)-2-methyl-1H-imidazol-5-yl)carbamoyl)furan-2-sulfonamide

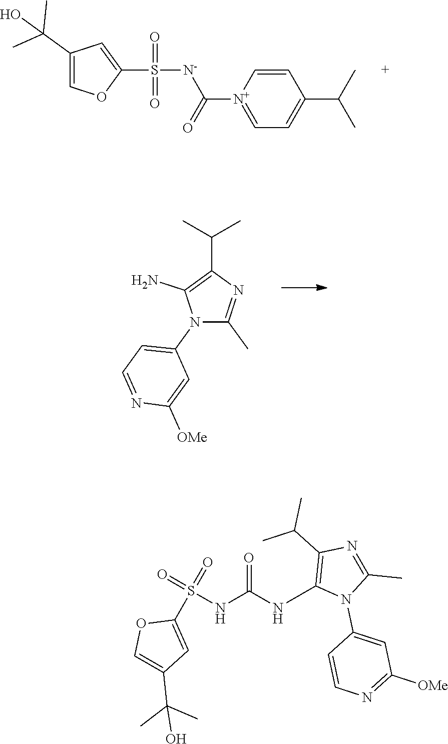 [SMILES: CC(C)c1cc[n+](C(=O)[N-]S(=O)(=O)c2cc(C(C)(C)O)co2)cc1 ; COc1cc(-n2c(C)nc(C(C)C)c2N)ccn1 ; COc1cc(-n2c(C)nc(C(C)C)c2NC(=O)NS(=O)(=O)c2cc(C(C)(C)O)co2)ccn1]

A mixture of 4-isopropyl-1-(2-methoxypyridin-4-yl)-2-methyl-1H-imidazol-5-amine (Intermediate R40) (178 mg, 722.67 μmol, 1 eq) and ((4-(2-hydroxypropan-2-yl)furan-2-yl)sulfonyl)(4-isopropylpyridin-1-ium-1-carbonyl)amide (Example 46, step A) (255 mg, 722.67 μmol, 1 eq) in MeCN (₅ mL) was stirred at 70° C. for 2 hours under N₂. The reaction mixture was purified directly by reversed phase flash is chromatography (0.1% NH₃.H₂O-MeCN) and then further purified by reversed phase prep HPLC (column: Xtimate C18, 150 mm×25 mm×5 μm; mobile phase [A: water (0.05% ammonium hydroxide v/v); B: MeCN]; B %: 0%-24%, 10 min) to give the title compound (24.92 mg, 7% yield over two steps, 100% purity on LCMS) as a white solid.

¹H NMR (400 MHz, DMSO-d₆) δ 8.27-8.19 (m, 1H), 7.68-7.45 (m, 1H), 7.39 (s, 1H), 7.04-6.77 (m, 2H), 6.65-6.53 (m, 1H), 4.93 (s, 1H), 3.87 (s, 3H), 2.80-2.69 (m, 1H), 2.22 (s, 3H), 1.33 (s, 6H) and 1.10 (s, 6H).

LCMS: m/z 478.3 (M+H)⁺(ES⁺).

### Example 49

### 1-(5-Isopropyl-2-methyl-3-(4-pyridyl)imidazol-4-yl)-3-(methyl-(1-methylpyrrolidin-3-yl)sulfamoyl)urea

### Step A: (4-(Dimethylamino)pyridin-1-ium-1-carbonyl)(N-methyl-N-(1-methylpyrrolidin-3-yl)sulfamoyl)amide

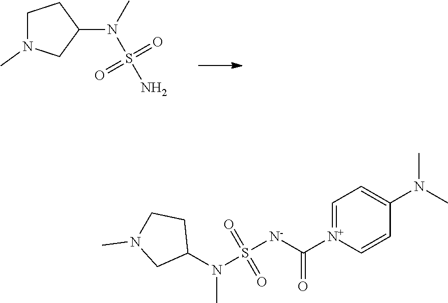

A solution of N,N-dimethylpyridin-4-amine (366 mg, 3.00 mmol, 2 eq) and 1-methyl-3-[methyl(sulfamoyl)amino]pyrrolidine (Intermediate L9) (0.29 g, 1.50 mmol, 1 eq) in MeCN (8 mL) was stirred at 20° C. for 30 minutes. Then diphenyl carbonate (353 mg, 1.65 mmol, 1.1 eq) was added. The resulting mixture was stirred at 20° C. for 12 hours. The mixture (theoretical amount: 0.53 g, crude) was used directly in the next step.

### Step B: 1-(5-Isopropyl-2-methyl-3-(4-pyridyl)imidazol-4-yl)-3-(methyl-(1-methylpyrrolidin-3-yl)sulfamoyl)urea

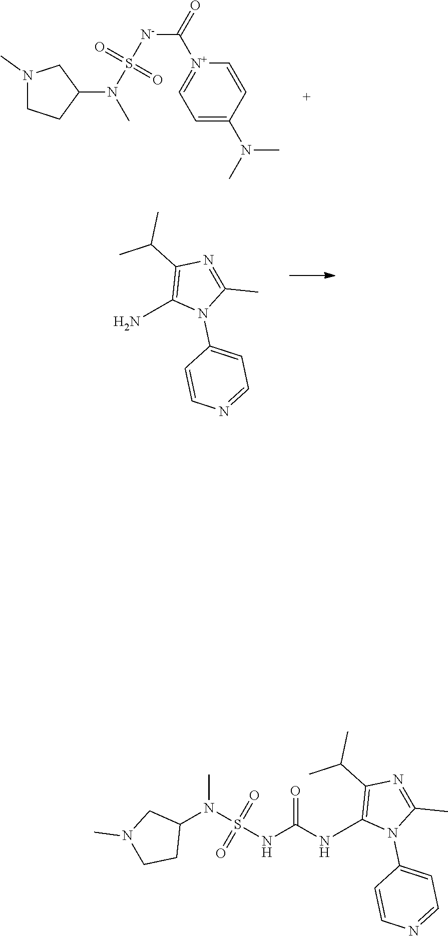

To a mixture of 4-isopropyl-2-methyl-1-(pyridin-4-yl)-1H-imidazol-5-amine (Intermediate R39) (0.2 g, 791.32 μmol, 1 eq, HCl salt) in MeCN (1 mL) was added a solution of (4-(dimethylamino)pyridin-1-ium-1-carbonyl)(N-methyl-N-(1-methylpyrrolidin-3-yl)sulfamoyl)amide (the reaction mixture of step A) in MeCN (8 mL). The resulting mixture was heated to 70° C. and stirred for 30 minutes under N₂. Then the reaction mixture was concentrated in vacuo. The residue was purified by reversed phase flash chromatography (0.1% NH₃.H₂O-MeCN) and then further purified by prep HPLC (column: Waters XBridge C18, 150 mm×25mm×5 μm; mobile phase [A: water (10 mM NH₄HCO₃), B: MeCN]; B %: 1%-15%, 10 minutes) to give the title compound (25.13 mg, 7% yield over two steps, 100% purity on LCMS) as a white solid.

¹H NMR (400 MHz, CD₃OD) δ 8.70 (d, J=6.0 Hz, 2H), 7.50-7.48 (m, 2H), 4.48-4.44 (m, 1H), 3.30-2.92 (m, 5H), 2.74(s, 3H), 2.63 (s, 3H), 2.29 (s, 3H), 2.15-1.98 (m, 2H), 1.27 (d, J=6.8 Hz, 6H). 2 ×NH were missing.

LCMS: m/z 436.1 (M+H)⁺(ES⁺).

### Example 50

### N-((4-Isopropyl-2-methyl-1-(pyridin-4-yl)-1H-imidazol-5-yl)carbamoyl)benzenesulfonamide

### Step A: (4-(Dimethylamino)pyridin-1-ium-1-carbonyl)(phenylsulfonyl)amide

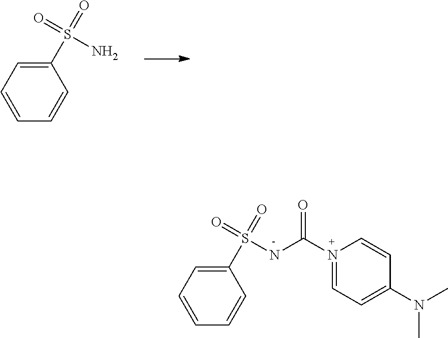

A solution of benzenesulfonamide (125 mg, 795.22 μmol, 1 eq) and N,N-dimethylpyridin-4-amine (194 mg, 1.59 mmol, 2 eq) in MeCN (3 mL) was stirred at 25° C. for 3o minutes. Then diphenyl carbonate (187 mg, 874.74 μmol, 1.1 eq) was added. The resulting mixture was stirred at 25° C. for 12 hours. The reaction mixture (theoretical amount: 242 mg, crude) was used directly in the next step.

### Step B: N-((4-Isopropyl-2-methyl-1-(pyridin-4-yl)-1H-imidazol-5-yl)carbamoyl)benzenesulfonamide

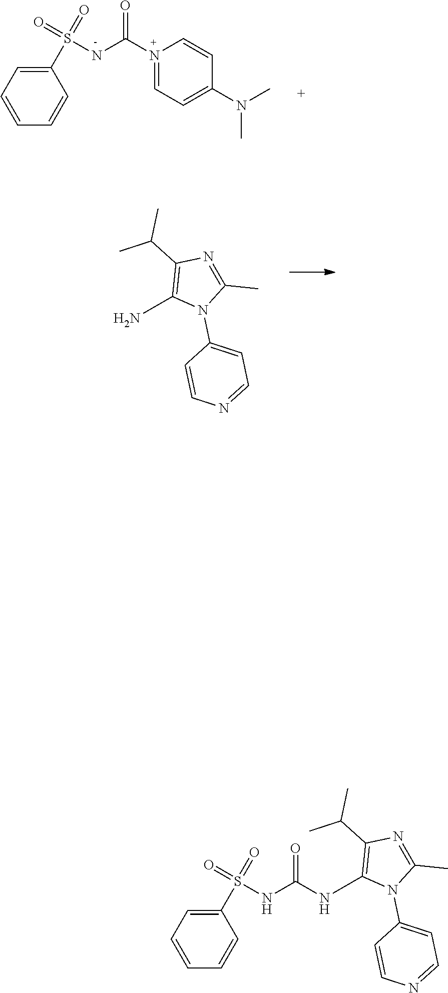

To a solution of 4-isopropyl-2-methyl-1-(pyridin-4-yl)-1H-imidazol-5-amine (197 mg, 780.00 μmol, 1 eq, HCl salt) (Intermediate R39) in MeCN (2 mL) was added a solution of (4-(dimethylamino)pyridin-1-ium-1-carbonyl)(phenylsulfonyl)amide (the reaction mixture of step A) in MeCN (3 mL). The resulting mixture was stirred at 70° C. for 45 minutes under N₂. Then the reaction mixture was concentrated and the residue was purified by reversed phase flash chromatography (0.1% NH₃.H₂O-MeCN) and then further purified by prep HPLC (column: Xtimate C18, 150 mm×25 mm×5 μm; mobile phase [A: water (0.05% ammonium hydroxide v/v), B: MeCN]; B %: 0%-30%, 10 minutes) to give the title compound (17.42 mg, 6% yield over two steps, 99.8% purity on HPLC) as a white solid.

¹H NMR (400 MHz, DMSO-d₆) δ 8.60-8.45 (m, 2H), 7.70-7.47 (m, 6H), 7.26-6.90 (m, 2H), 2.68-2.65 (m, 1H), 2.19 (s, 3H) and 1.09 (d, 6H). 1×NH was missing.

LCMS: m/z 400.1 (M+H)⁺(ES⁺).

### Example 51

### N-((6-Methyl-5-(2-((1-methylpiperidin-4-yl)oxy)pyridin-4-yl)-2,3-dihydro-1H-inden-4-yl)carbamoyl)benzenesulfonimidamide

### Step A

### N-((6-Methyl-5-(2-((1-methylpiperidin-4-yl)oxy)pyridin-4-yl)-2,3-dihydro-1H-inden-4-yl)carbamoyl)benzenesulfinamide

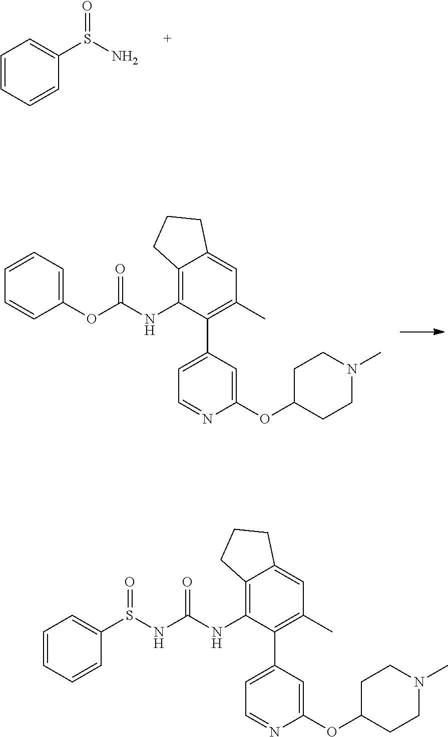 [SMILES: NS(=O)c1ccccc1 ; Cc1cc2c(c(NC(=O)Oc3ccccc3)c1-c1ccnc(OC3CCN(C)CC3)c1)CCC2 ; Cc1cc2c(c(NC(=O)NS(=O)c3ccccc3)c1-c1ccnc(OC3CCN(C)CC3)c1)CCC2]

To a solution of benzenesulfinamide (Intermediate L10) (50 mg, 354.13 μmol, 1 eq) in THF (1 mL) was added with t-BuONa (102 mg, 1.06 mmol, 3 eq) at 25° C. The reaction mixture was stirred at 25° C. for 30 minutes. Then phenyl (6-methyl-5-(2-((1-methylpiperidin-4-yl)oxy)pyridin-4-yl)-2,3-dihydro-1H-inden-4-yl)carbamate (Intermediate R41) (202 mg, 354.13 μmol, 1 eq, TFA salt) was added. The resulting mixture was stirred at 25° C. for 5 hours. Then the reaction mixture was concentrated in vacuo. The residue was purified by reversed phase flash chromatography (0.1% NH₃.H₂O-MeCN) to give the title compound (120 mg, 59% yield, 88% purity on LCMS) as a white solid.

¹H NMR (400 MHz, DMSO-d₆) δ 9.57-9.27 (m, 1H), 8.31-8.17 (m, 1H), 7.71-7.42 (m, 5H), 7.24-7.07 (m, 1H), 6.82-6.65 (m, 1H), 6.51 (s, 1H), 5.11-4.93 (m, 1H), 3.48-3.41 (m, 2H), 2.99-2.68 (m, 5H), 2.24-1.96 (m, 11H) and 1.89-1.61 (m, 2H). 1×NH was missing.

LCMS: m/z 505.3 (M+H)⁺(ES⁺).

### Step B

### N-((6-Methyl-5-(2-((1-methylpiperidin-4-yl)oxy)pyridin-4-yl)-2,3-dihydro-1H-inden-4-yl)carbamoyl)benzenesulfonimidamide

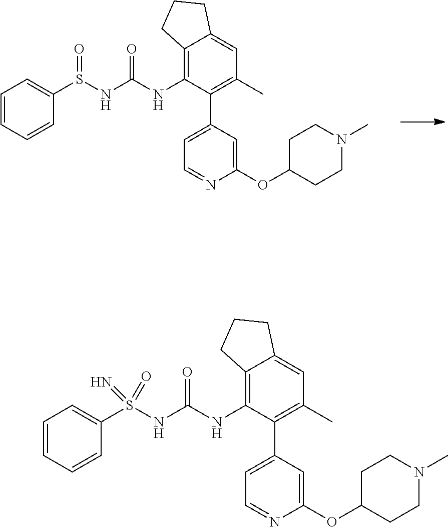 [SMILES: Cc1cc2c(c(NC(=O)NS(=O)c3ccccc3)c1-c1ccnc(OC3CCN(C)CC3)c1)CCC2 ; Cc1cc2c(c(NC(=O)NS(=N)(=O)c3ccccc3)c1-c1ccnc(OC3CCN(C)CC3)c1)CCC2]

To a solution of N-((6-methyl-5-(2-((1-methylpiperidin-4-yl)oxy)pyridin-4-yl)-2,3-dihydro-1H-inden-4-yl)carbamoyl)benzenesulfinamide (0.1 g, 198.16 μmol, 1 eq) in THF (1 mL) was added 1-chloro-1H-benzo[d][1,2,3]triazole (27 mg, 178.34 μmol, 0.9 eq). The reaction mixture was stirred at 25° C. for 30 minutes. Then the reaction mixture was added into a solution of NH₃/THF (5 mL) at −70° C.; NH₃ was bubbled into THF for 5 minutes to afford the NH₃/THF solution. After addition, the mixture was stirred at −70° C. for 30 minutes. Then the reaction mixture was concentrated in vacuo. The residue was purified by prep-TLC (SiO₂, DCM:methanol, 10:1) and then further purified by prep HPLC (column: Phenomenex luna C18, 150 mm×25 mm×10 μm; mobile phase [A: water (0.1% TFA); B: MeCN]; B%: 22%-42%, 10 minutes) to give the title compound (21.45 mg, 17% yield, 100% purity on LCMS, TFA salt) as a white solid.

¹H NMR (400 MHz, CDCl₃) δ 8.22-8.10 (m, 1H), 8.02-7.80 (m, 2H), 7.68-7.46 (m, 3H), 7.10 (d, 1H), 6.83-6.62 (m, 2H), 5.45-5.21 (m, 1H), 3.93-3.40 (m, 2H), 3.38-3.01 (m, 2H), 3.00-2.71 (m, 7H), 2.49-2.19 (m, 4H) and 2.17-1.96 (m, 5H). 3×NHs were missing.

LCMS: m/z 520.1 (M+H)⁺(ES⁺).

### Example 52

### N-((6-Methyl-5-(2-((1-methylpiperidin-4-yl)oxy)pyridin-4-yl)-2,3-dihydro-1H-inden-4-yl)carbamoyl)methanesulfonimidamide

### Step A: N-((6-Methyl-5-(2-((1-methylpiperidin-4-yl)oxy)pyridin-4-yl)-2,3-dihydro-1H-inden-4-yl)carbamoyl)methanesulfinamide

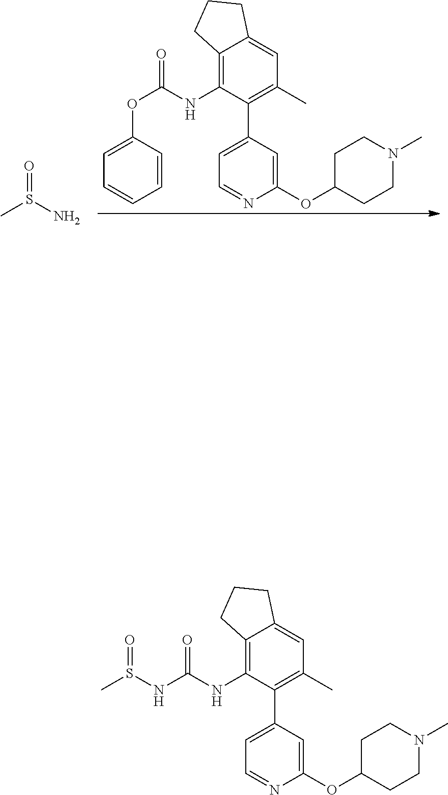

Sodium tert-butoxide (134 mg, 1.40 mmol, 1.6 eq) was added into a mixture of methanesulfinamide (Intermediate L11) (103 mg, 1.31 mmol, 1.5 eq) in THF (2 mL) at 20° C. The reaction mixture was stirred at 20° C. for 30 minutes. Then phenyl (6-methyl-5-(2-(((1-methylpiperidin-4-yl)oxy)pyridin-4-yl)-2,3-dihydro-1H-inden-4-yl)carbamate (Intermediate R41) (400 mg, 874.20 μmol, 1 eq) was added at 20° C. and the resulting mixture was stirred at 20° C. for 30 minutes. The reaction mixture was poured into ice-water (30 mL). The aqueous phase was extracted with ethyl acetate (3×20 mL). The combined organic phases were washed with brine (20 mL), dried over anhydrous sodium sulfate, filtered and concentrated in vacuo. The residue was purified by reversed phase flash chromatography (0.1% NH₃.H₂O-MeCN) to afford the title compound (150 mg, 26% yield, 68% purity on LCMS) as a white solid.

¹H NMR (400 MHz, CDCl₃) δ 8.17 (d, 1H), 7.14 (d, 1H), 6.71-6.65 (m, 1H), 6.54 (d, 1H), 5.08-5.04 (m, 1H), 2.97 (t, 2H), 2.87 (t, 2H), 2.75-2.73 (m, 2H), 2.64 (s, 3H), 2.33-2.27 (m, 5H), 2.15-2.07 (m, 7H) and 1.86-1.73 (m, 2H). 2×NHs were missing.

LCMS: m/z 443.4 (M+H)⁺(ES⁺).

### Step B: N-((6-Methyl-5-(2-((1-methylpiperidin-4-yl)oxy)pyridin-4-yl)-2,3 -dihydro-1H-inden-4-yl)carbamoyl)methanesulfonimidamide

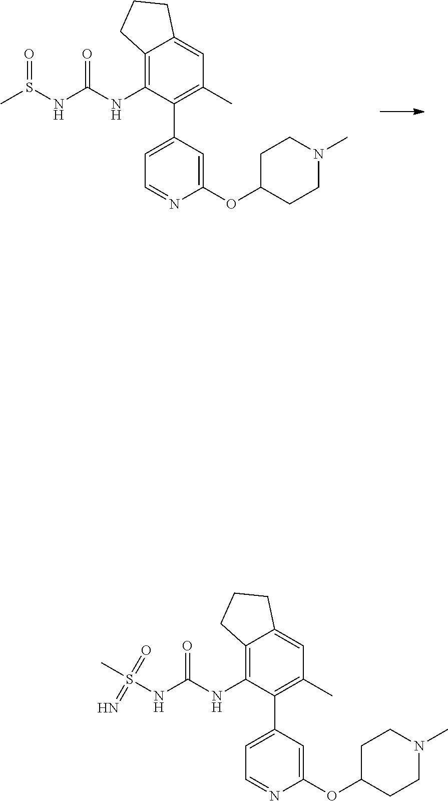

To a solution of N-((6-methyl-5-(2-((1-methylpiperidin-4-yl)oxy)pyridin-4-yl)-2,3-dihydro-1H-inden-4-yl)carbamoyl)methanesulfinamide (250 mg, 564.88 μmol, 1 eq) in THF (5 mL) was added 1-chloro-1H-benzo[d][1,2,3]triazole (78 mg, 508.39 μmol, 0.9 eq) at 20° C. The reaction mixture was stirred for 30 minutes at 20° C. Then the reaction mixture was added into a solution of NH₃/THF at −78° C.; NH₃ gas (15 psi) was bubbled into THF (5 mL) for 5 minutes to afford the NH₃/THF solution. The resulting mixture was stirred at −78° C. for 20 minutes, and then warmed to 20° C. and stirred for 2 hours. Then the reaction mixture was concentrated in vacuo. The residue was purified by prep HPLC (column: Xtimate C18, 150 mm×40 mm×10 μm; mobile phase [A: water (0.05% ammonium hydroxide v/v); B: MeCN]; B %: 19%-49%, 10 minutes) to afford the title compound (2.19 mg, 1% yield, 99.8% purity on LCMS) as a yellow solid.

¹H NMR (400 MHz, CD₃OD) δ 8.12 (d, 1H), 7.09 (s, 1H), 6.82 (d, 1H), 6.69 (s, 1H), 5.14-5.12 (m, 1H), 3.21 (s, 3H), 3.03-3.01 (m, 2H), 2.95 (t, 2H), 2.88 (t, 2H), 2.83-2.57 (m, 2H), 2.55 (s, 3H) and 2.14-1.92 (m, 9H). 3×NHs were missing.

LCMS: m/z 458.3 (M+H)⁺(ES⁺).

### Example 53

### 1-(N-Cyano-S-methyl-sulfonimidoyl)-3-(6-methyl-5-(2-((1-methyl-4-piperidyl)oxy)-4-pyridyl)indan-4-yl)urea

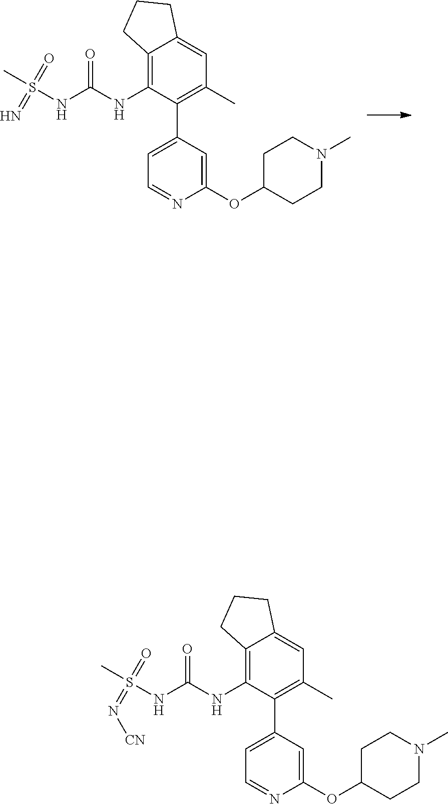

To a mixture of N-((6-methyl-5-(2-((1-methylpiperidin-4-yl)oxy)pyridin-4-yl)-2,3-dihydro-1H-inden-4-yl)carbamoyl)methanesulfonimidamide (Example 52) (30 mg, 65.56 μmol, 1 eq) and triethylamine (27 mg, 262.24 μmol, 4 eq) in DMF (1 mL) was added cyanic bromide (14 mg, 131.12 μmol, 2 eq) at 25° C. The reaction mixture was stirred at 25° C. for 12 hours, and then quenched with water (0.5 mL) and concentrated in vacuo. The residue was purified by prep HPLC (column: Phenomenex Gemini-NX C18, 75 mm×30 mm×3 μm; mobile phase [A: water (0.1% TFA), B: MeCN]; B %: 20%-30%, 7 minutes) to afford the title compound (21.8 mg, 54% yield, 97.6% purity on HPLC, TFA salt) as yellow oil.

¹H NMR (400 MHz, DMSO-d6+D₂O) δ 8.16 (t, 1H), 7.09 (s, 1H), 6.79 (t, 1H), 6.63 (d, 1H), 5.27-5.13 (m, 1H), 3.48-3.45 (m, 1H), 3.35-3.32 (m, 1H), 3.27-3.12 (m, 5H), 2.88 (t, 2H), 2.81-2.75 (m, 5H), 2.34-2.30 (m, 1H), 2.18-2.13 (m, 1H), 3.05-1.96 (m, 6H) and 1.83-1.75 (m, 1H). 2×NHs were missing.

LCMS: m/z 483.2 (M+H)⁺(ES⁺).

### Examples

### Biological Studies

NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 6o-65; Mattia Cocco et al., Journal of Medicinal Chemistry, 2014, 57(24),10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

THP-1 Cells: Culture and Preparation

THP-1 cells (ATCC #TIB-202) were grown in RPMI containing L-glutamine (Gibco #11835) supplemented with imM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hrs at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Nates were read in an Envision reader at Ex 560 nm and Em 590 nm
9. IC₅₀ data is fitted to a non-linear regression equation (log inhibitor vs response-variable slope 4-parameters)

96-well Nate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950 (10 uM)
Low Drug free control
Compound 8-point half-log dilution

The results of the pyroptosis assay are summarised in Table 1 below as THP IC₅₀.

Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Nate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300 ×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

For comparison, three compounds outside the scope of the claims are included in Table 1:

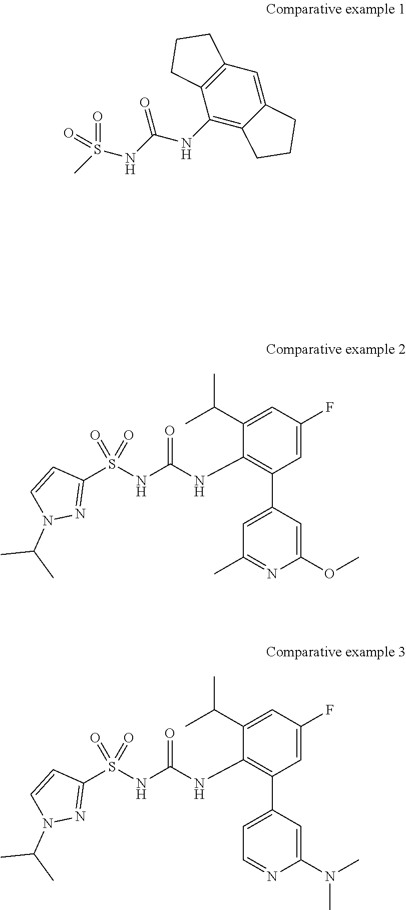

TABLE 1
NLRP3 inhibitory activity

| Example No | THP IC₅₀ | HWB IC₅₀ |
|---|---|---|
| 1 | ++++ | ++++ |
| 2 | ++++++ | +++++ |
| 3 | ++++++ | ++++ |
| 4 | ++++++ | +++++ |
| 5 | ++++++ | +++++ |
| 6 | ++++++ | ++++++ |
| 7 | +++++ | ++++ |
| 8 | +++++ | ++++ |
| 10 | ++++++ | +++++ |
| 11 | ++++++ | +++ |
| 12 | ++++++ | ++++ |
| 13 | ++++++ | ++++ |
| 14 | ++++++ | ++++ |
| 15 | ++++++ | ++++ |
| 16 | ++++++ | +++ |
| 18 | ++++++ | ++ |
| 19 | ++++++ | ++++ |
| 20 | +++++ | +++++ |
| 21 | ++++++ | ++++++ |
| 22 | ++++++ | ++++++ |
| 23 | ++++++ | ++++++ |
| 24 | ++++++ | +++++ |
| 25 | ++++++ | +++++ |
| 26 | ++++++ | ++++++ |
| 27 | ++++++ | ++++++ |
| 28 | ++++++ | ++++++ |
| 29 | ++++++ | +++ |
| 31 | ++++ | +++ |
| 33 | ++++ | +++ |
| 34 | ++++++ | +++ |
| 35 | ++++++ | ++++ |
| 36 | ++++ | ++++ |
| 38 | +++++ | +++ |
| 40 | ++++++ | +++++ |
| 42 | ++++++ | +++++ |
| 44 | ++++++ | +++++ |
| 45 | ++++++ | ++++ |
| 46 | ++++++ | ++++++ |
| 47 | +++++ | +++++ |
| 48 | +++++ | +++++ |
| 49 | + | ND |
| 51 | ++++++ | +++ |
| 52 | ++++++ | ++++ |
| comp ex 1 | +++ | inactive |
| comp ex 2 | ++ | ND |
| comp ex 3 | ++ | ND |

(≤0.25 μM = ‘++++++’, ≤0.5 μM = ‘+++++’, ≤1 μM = ‘++++’, ≤2 μM = ‘+++’, ≤5 μM = ‘++’, ≤10 μM = ‘+’, >10 μM = ‘inactive’, not determined = ‘ND’).

As is evident from the results presented in Table 1, surprisingly in spite of the structural differences versus the prior art compounds, the compounds of the invention show high levels of NLRP3 inhibitory activity in the pyroptosis assay and in particular in the human whole blood assay.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 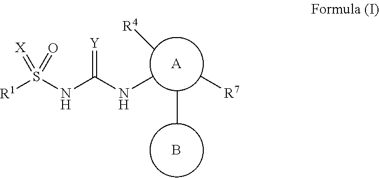
or a pharmaceutically acceptable salt, solvate or prodrug thereof, wherein:
A is a phenyl or 5- or 6-membered heteroaryl group, wherein A is substituted in the a position with B, in the β position with R⁷ and in the α′ position with R⁴, and wherein A is optionally further substituted;
B is a phenyl, 5- or 6-membered heteroaryl, or 4- to 6-membered saturated heterocyclic group, wherein B is optionally substituted;
X is O, NH or N(CN);
Y is O or S;
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
either R⁴ is monovalent, and attached to A in the α′ position, and selected from C₁-C₄ alkyl, C₃-C₆ cycloalkyl and phenyl, all optionally halo-substituted and/or optionally substituted with one or two substituents independently selected from oxo, —OH, —O(C₁-C₄ alkyl) and —O(C₁-C₄ haloalkyl);
or R⁴ is divalent, and attached to A in the α′ and β′ positions, and selected from —CH₂CH₂CH₂—, —CH═CHCH₂—, —CH₂CH═CH—, —CH₂CH₂O—, —OCH₂CH₂—, —CH₂CH₂CH₂CH₂—and —CH═CH—CH═CH—, all optionally halo-substituted and/or optionally substituted with one or two substituents independently selected from oxo, —OH, —O(C₁-C₄ alkyl) and —O(C₁-C₄ haloalkyl);
R⁷ is C₁-C₄ alkyl, C₁-C₄ haloalkyl, C₃-C₆ cycloalkyl, C₃-C₆ halocycloalkyl, —O(C₁-C₄ alkyl), —O(C₁-C₄ haloalkyl) or halogen;
R²⁰ is a bond, —NH—, —NMe—, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

2. The compound or a pharmaceutically acceptable salt, solvate or prodrug thereof as claimed in claim 1, wherein B is a pyridinyl group which is optionally substituted.

3. The compound or a pharmaceutically acceptable salt, solvate or prodrug thereof as claimed in claim 1, wherein the compound is of formula (IA): 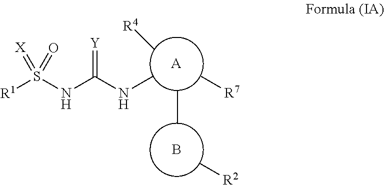
wherein:
A is a phenyl or 5- or 6-membered heteroaryl group, wherein A is substituted in the a position with B, in the β position with R⁷ and in the α′ position with R⁴, and wherein A is optionally further substituted;
B is a phenyl, 5- or 6-membered heteroaryl, or 4- to 6-membered saturated heterocyclic group, wherein B is substituted with R², and wherein B is optionally further substituted;
X is O, NH or N(CN);
Y is O or S;
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R² is hydrogen, halo, cyano, C₁-C₄ alkyl, C₁-C₄ haloalkyl, —R⁸—OH, —R⁸—O(C₁-C₄ alkyl), —R⁸—O(C₁-C₄ haloalkyl), —O—R¹⁰—OH, —O—R¹⁰—O(C₁-C₄ alkyl), —O—R¹⁰—O(C₁-C₄ haloalkyl), —R⁸—NH₂, —R⁸—NH(C₁-C₄ alkyl), —R⁸—NH(C₁-C₄ haloalkyl), —R⁸—N(C₁-C₄ alkyl)₂, —R⁸—N(C₁-C₄ alkyl)(C₁-C₄ haloalkyl), —R⁸—N(C₁-C₄ haloalkyl)₂, —R¹¹, —OR¹¹ or —O—R¹⁰—R¹¹;
either R⁴ is monovalent, and attached to A in the α′ position, and selected from C₁-C₄ alkyl, C₃-C₆ cycloalkyl and phenyl, all optionally halo-substituted and/or optionally substituted with one or two substituents independently selected from oxo, —OH, —O(C₁-C₄ alkyl) and —O(C₁-C₄ haloalkyl);
or R⁴ is divalent, and attached to A in the α′ and β′ positions, and selected from —CH₂CH₂CH₂—, —CH═CHCH₂—, —CH₂CH═CH—, —CH₂CH₂O—, —OCH₂CH₂—, —CH₂CH₂CH₂CH₂— and —CH═CH—CH═CH—, all optionally halo-substituted and/or optionally substituted with one or two substituents independently selected from oxo, —OH, —O(C₁-C₄ alkyl) and —O(C₁-C₄ haloalkyl);
R⁷ is C₁-C₄ alkyl, C₁-C₄ haloalkyl, C₃-C₆ cycloalkyl, C₃-C₆ halocycloalkyl, —O(C₁-C₄ alkyl), —O(C₁-C₄ haloalkyl) or halogen;
R⁸ is a bond, C₁-C₄ alkylene or C₁-C₄ haloalkylene;
R¹⁰ is C₁-C₄ alkylene or C₁-C₄ haloalkylene;
R¹¹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from cyano, C₁-C₄ alkyl, C₁-C₄ haloalkyl, C₃-C₄ cycloalkyl, C₂-C₄ alkenyl, C₂-C₄ haloalkenyl, phenyl, benzyl, —OH, —O(C₁-C₄ alkyl), —O(C₁-C₄ haloalkyl), —NH₂, —NH(C₁-C₄ alkyl), —NH(C₁-C₄ haloalkyl), —N(C₁-C₄ alkyl)₂, —N(C₁-C₄ alkyl)(C₁-C₄ haloalkyl) and —N(C₁-C₄ haloalkyl)₂;
R²⁰ is a bond, —NH—, —NMe-, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

4. The compound or a pharmaceutically acceptable salt, solvate or prodrug thereof as claimed in claim 1, wherein B is a pyridinyl group, substituted with R², and optionally further substituted.

5. The compound or a pharmaceutically acceptable salt, solvate or prodrug thereof as claimed in claim 1, wherein A is a phenyl or imidazolyl group, wherein A is substituted in the a position with B, in the β position with R⁷ and in the α′ position with R⁴, and wherein A is optionally further substituted.

6. The compound or a pharmaceutically acceptable salt, solvate or prodrug thereof as claimed in claim 1, wherein Y is O.

7. The compound or a pharmaceutically acceptable salt, solvate or prodrug thereof as claimed in claim 1, wherein
either R⁴ is monovalent, and attached to A in the α′ position, and selected from isopropyl, cyclopentyl, cyclohexyl and phenyl;
or R⁴ is divalent, and attached to A in the α′ and β′ positions, and selected from —CH₂CH₂CH₂—, —CH₂CH₂O—and —OCH₂CH₂—.

8. The compound or a pharmaceutically acceptable salt, solvate or prodrug thereof as claimed in claim 1, wherein the compound is of formula (II): 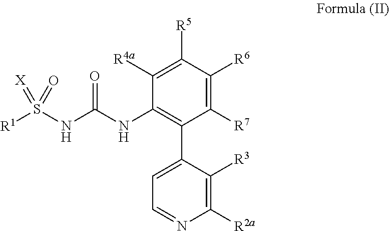
wherein:
X is O, NH or N(CN);
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R^{2a} is hydrogen, halo, cyano, C₁-C₄ alkyl, C₁-C₄ haloalkyl, —O(C₁-C₄ alkyl), —O(C₁-C₄ haloalkyl), —O-(alkoxyalkyl), —OR⁹ or —OCH₂—R⁹;
R³ is hydrogen or methyl;
R^{4a} is C₁-C₄ alkyl, C₃-C₆ cycloalkyl or phenyl, all optionally halo-substituted;
R⁵ is hydrogen; or
R^{4a} and R⁵ together form —CH₂CH₂CH₂—, —CH₂CH₂O—or —OCH₂CH₂—, all optionally halo-substituted;
R⁶ is hydrogen, halogen or cyano;
R⁷ is C₁-C₄ alkyl, C₁-C₄ haloalkyl, C₃-C₆ cycloalkyl, C₃-C₆ halocycloalkyl or halogen;
R⁹ is a C₃-C₆ cycloalkyl or 4- to 6-membered saturated heterocyclic group, wherein the cycloalkyl or heterocyclic group is optionally halo-substituted and/or optionally substituted with one, two or three substituents independently selected from C₁-C₄ alkyl, C₂-C₄ alkenyl, phenyl, benzyl, —OH, —O(C₁-C₄ alkyl), —NH(C₁-C₄ alkyl) and —N(C₁-C₄ alkyl)₂;
R²⁰ is a bond, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

9. The compound or a pharmaceutically acceptable salt, solvate or prodrug thereof as claimed in claim 8, wherein:
either R⁵ is hydrogen and R^{4a} is isopropyl, cyclopentyl, cyclohexyl or phenyl;
or R^{4a} and R⁵ together form —CH₂CH₂CH₂—, —CH₂CH₂O— or —OCH₂CH₂—.

10. The compound or a pharmaceutically acceptable salt, solvate or prodrug thereof as claimed in claim 8, wherein R⁶ is hydrogen or fluoro.

11. The compound or a pharmaceutically acceptable salt, solvate or prodrug thereof as claimed in claim 1, wherein the compound is of formula (III): 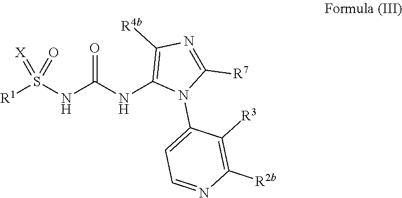
wherein:
X is O, NH or N(CN);
R¹ is a C₁-C₄ alkyl, C₂-C₄ alkenyl, —NH(C₁-C₄ alkyl), —N(C₁-C₄ alkyl)₂, or —R²⁰—R²¹ group, all optionally halo-substituted;
R^{2b} is hydrogen, halo, cyano, C₁-C₄ alkyl, C₁-C₄ haloalkyl, —O(C₁-C₄ alkyl) or —O(C₁-C₄ haloalkyl);
R³ is hydrogen or methyl;
R^{4b} is C₁-C₄ alkyl or C₁-C₄ haloalkyl;
R⁷ is C₁-C₄ alkyl, C₁-C₄ haloalkyl, C₃-C₆ cycloalkyl, C₃-C₆ halocycloalkyl or halogen;
R²⁰ is a bond, C₁-C₄ alkylene or C₁-C₄ haloalkylene;
R²¹ is a C₃-C₆ cycloalkyl, phenyl, 4- to 6-membered saturated heterocyclic, or 5- or 6-membered heteroaryl group, all optionally halo-substituted and/or optionally substituted with one or two substituents independently selected from cyano, C₁-C₄ alkyl, C₁-C₄ haloalkyl, C₂-C₄ alkenyl, C₂-C₄ haloalkenyl, —R²²—OH, —R²²—O(C₁-C₄ alkyl), —R²²—O(C₁-C₄ haloalkyl), —R²²—NH₂, —R²²—NH(C₁-C₄ alkyl), —R²²—NH(C₁-C₄ haloalkyl), —R²²—N(C₁-C₄ alkyl)₂, —R²²—N(C₁-C₄ alkyl)(C₁-C₄ haloalkyl), —R²²—N(C₁-C₄ haloalkyl)₂ and —R²²—R²³;
R²² is a bond, C₁-C₄ alkylene or C₁-C₄ haloalkylene; and
R²³ is a C₃-C₆ cycloalkyl or 4- to 6-membered saturated heterocyclic group, all optionally halo-substituted.

12. The compound or a pharmaceutically acceptable salt, solvate or prodrug thereof as claimed in claim 11, wherein R^{4b} is isopropyl, sec-butyl, isobutyl or t-butyl, all optionally halo-substituted.

13. The compound or a pharmaceutically acceptable salt, solvate or prodrug thereof as claimed in claim 1, wherein X is O.

14. The compound or a pharmaceutically acceptable salt, solvate or prodrug thereof as claimed in claim 1, wherein R¹ is C₁-C₄ alkyl, C₂-C₄ alkenyl, —NHMe, —NMe₂, —NHEt, —NEt₂ or —NMeEt, all optionally halo-substituted; or R¹ is a C₃-C₆ cycloalkyl, phenyl, pyrrolidinyl, piperidinyl, tetrahydrofuranyl, tetrahydropyranyl, furanyl, thiophenyl, pyrazolyl or imidazolyl group, all optionally halo-substituted and/or optionally substituted with one or two substituents independently selected from C₁-C₃ alkyl, —R²²—OH, —R²²—O(C₁-C₃ alkyl), —R²²—NH(C₁-C₃ alkyl), —R²²—N(C₁-C₃ alkyl)₂ and —R²²—R²³; wherein R²² is a bond or C₁-C₄ alkylene; and R²³ is a C₃-C₆ cycloalkyl, azetidinyl, pyrrolidinyl, piperidinyl, tetrahydrofuranyl or tetrahydropyranyl group.

15. The compound or a pharmaceutically acceptable salt, solvate or prodrug thereof as claimed in claim 3, wherein R² is hydrogen, halo, cyano, C₁-C₃ alkyl, C₁-C₃ haloalkyl, —R⁸—OH, —R⁸—O(C₁-C₃ alkyl), —R⁸—O(C₁-C₃ haloalkyl), —O—R₁₀—O(C₁-C₃ alkyl), —OR¹¹ or —O—R¹⁰—R¹¹; wherein
R⁸ is a bond or —CH₂—;
R¹⁰ is C₁-C₃ alkylene; and
R¹¹ is a cyclopropyl, cyclobutyl, cyclopentyl, cyclohexyl, pyrrolidinyl, piperidinyl, tetrahydrofuranyl or tetrahydropyranyl group, all optionally substituted with one or two substituents independently selected from fluoro, C₁-C₃ alkyl, C₂-C₃ alkenyl, phenyl, benzyl, —OH, —O(C₁-C₃ alkyl), —NH₂, —NH(C₁-C₃ alkyl) and —N(C₁-C₃ alkyl)₂.

16. The compound or a pharmaceutically acceptable salt, solvate or prodrug thereof as claimed in claim 1, wherein R⁷ is methyl, ethyl, cyclopropyl or fluoro.

17. The compound or a pharmaceutically acceptable salt, solvate or prodrug thereof as claimed in claim 1, selected from the group consisting of:

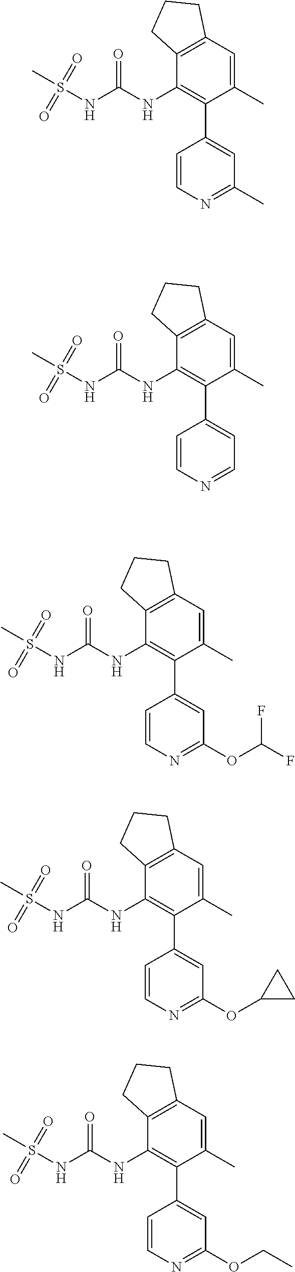

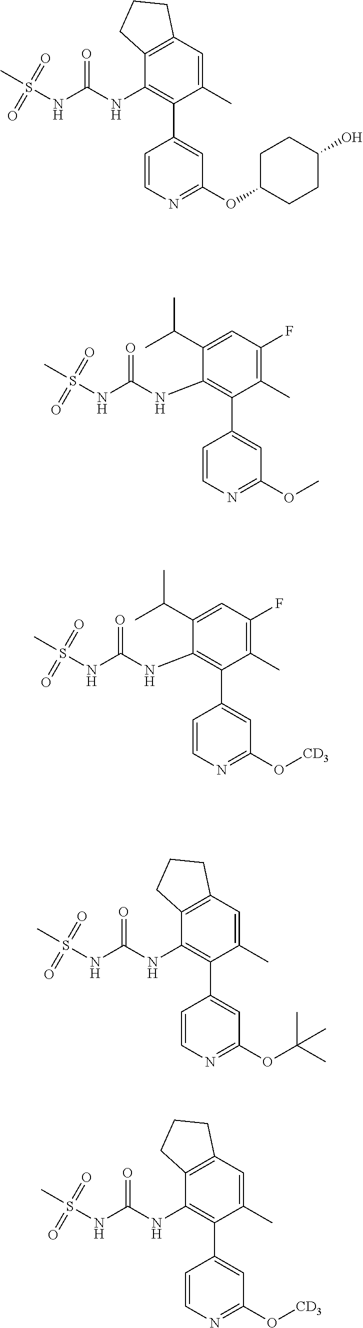

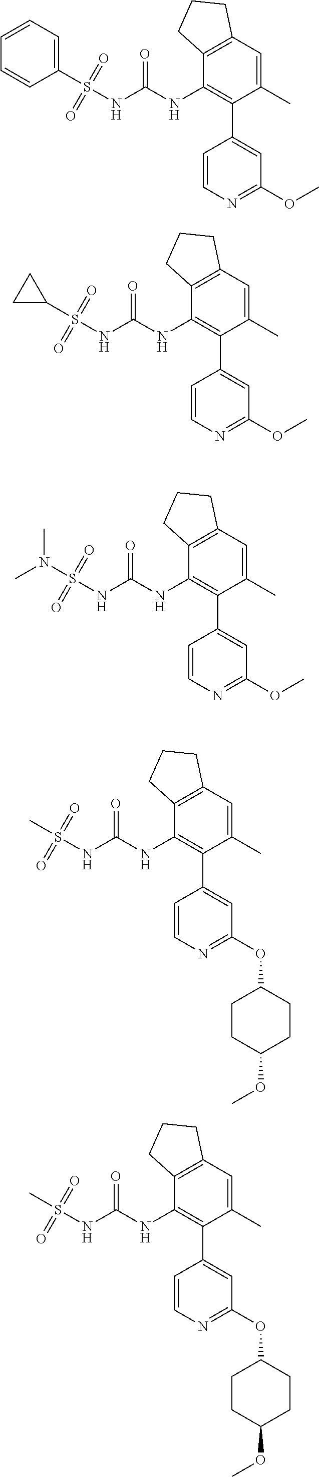

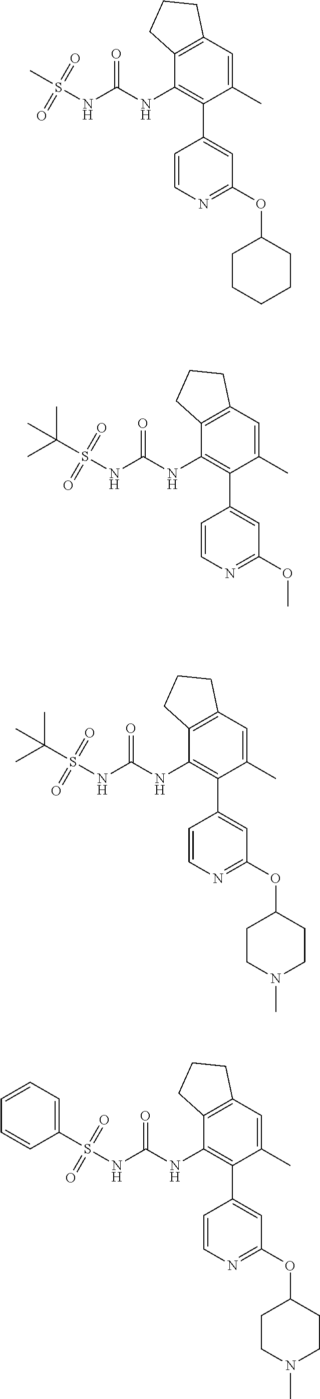

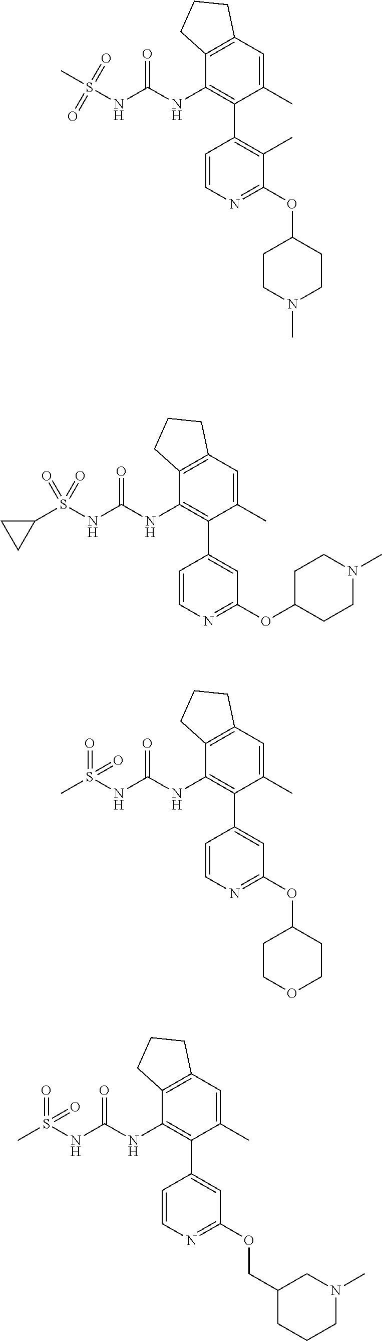

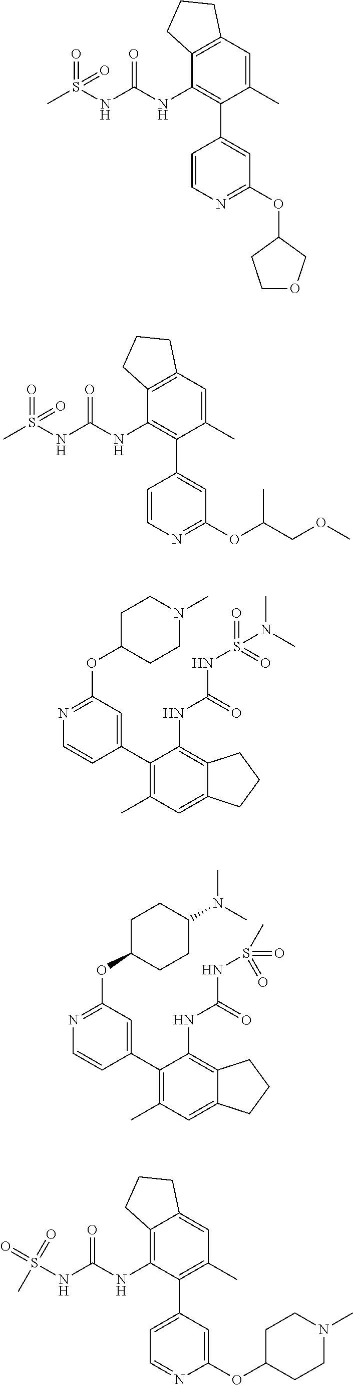

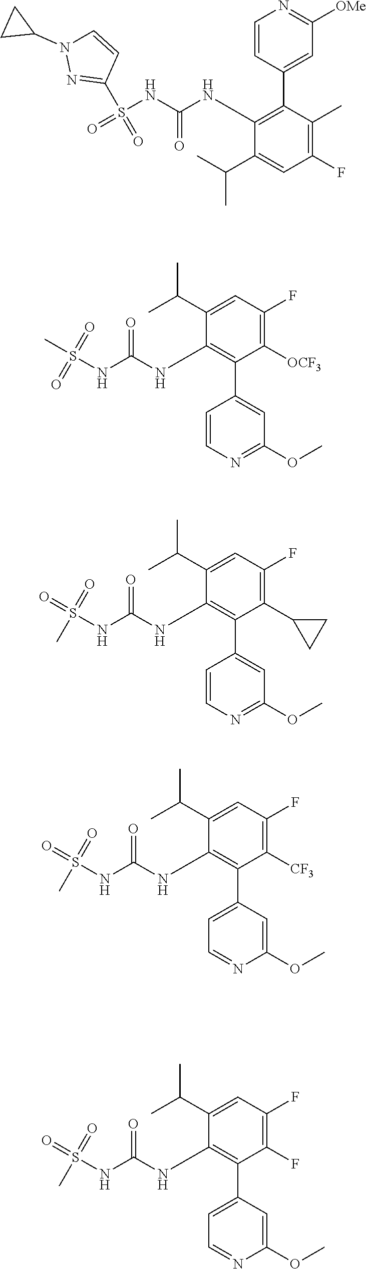

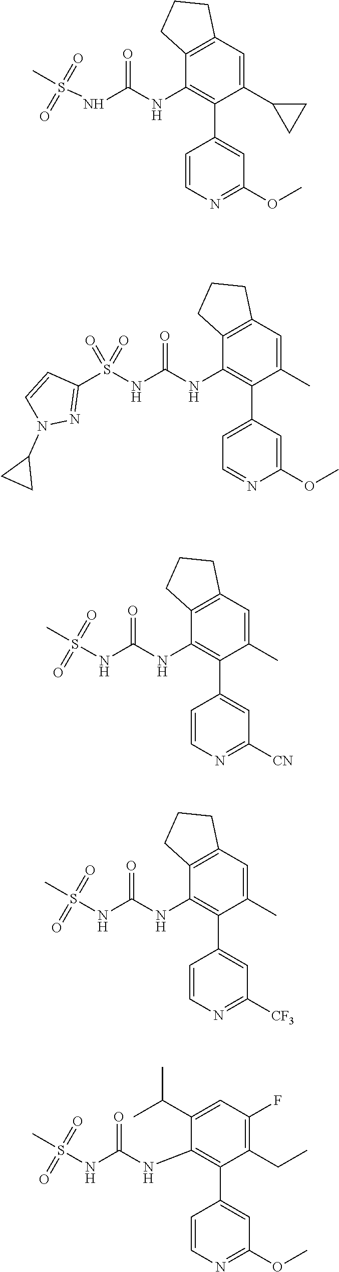

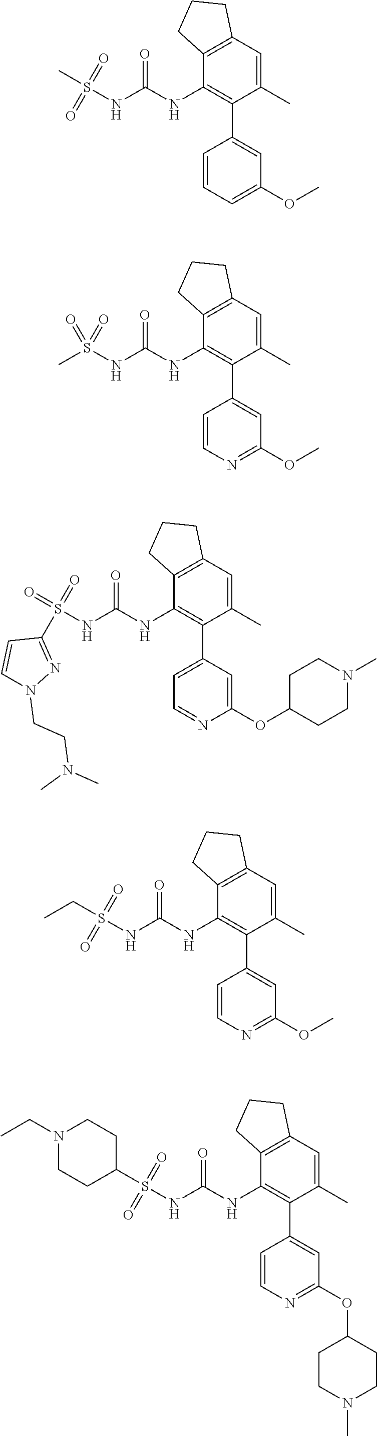

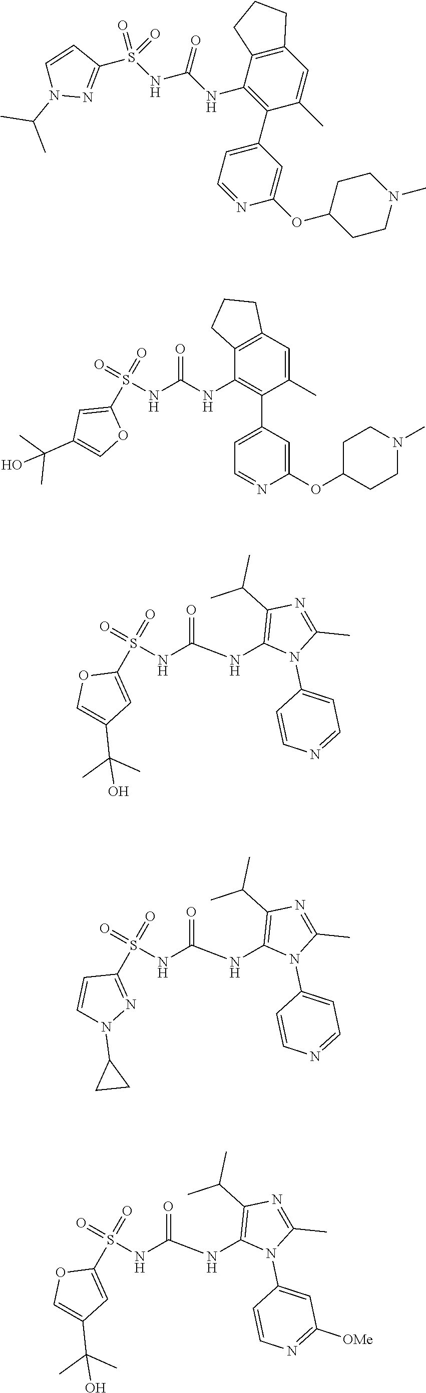 [SMILES: Cc1cc2c(c(NC(=O)NS(=O)(=O)c3ccn(C(C)C)n3)c1-c1ccnc(OC3CCN(C)CC3)c1)CCC2 ; Cc1cc2c(c(NC(=O)NS(=O)(=O)c3cc(C(C)(C)O)co3)c1-c1ccnc(OC3CCN(C)CC3)c1)CCC2 ; Cc1nc(C(C)C)c(NC(=O)NS(=O)(=O)c2cc(C(C)(C)O)co2)n1-c1ccncc1 ; Cc1nc(C(C)C)c(NC(=O)NS(=O)(=O)c2ccn(C3CC3)n2)n1-c1ccncc1 ; COc1cc(-n2c(C)nc(C(C)C)c2NC(=O)NS(=O)(=O)c2cc(C(C)(C)O)co2)ccn1]

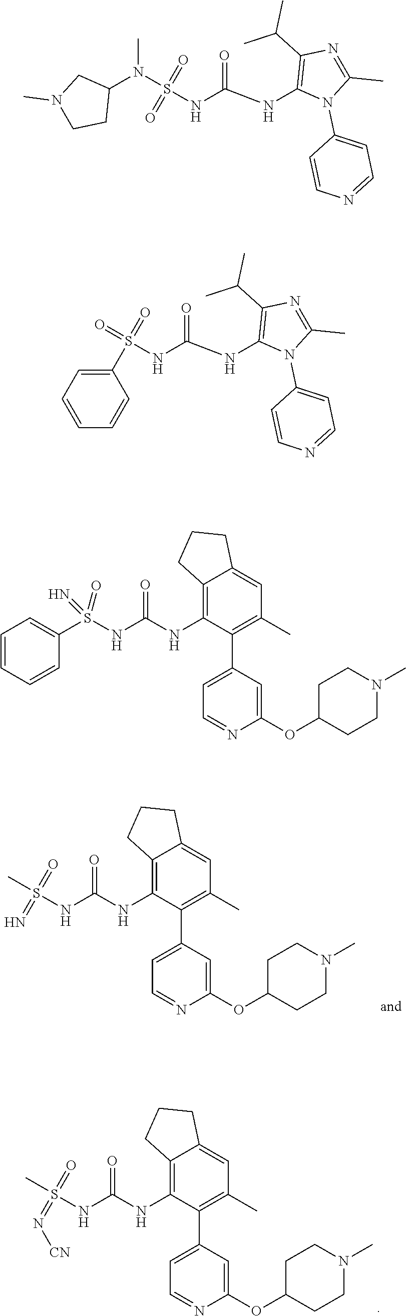

18. (canceled)

19. A pharmaceutical composition comprising a compound or a pharmaceutically acceptable salt, solvate or prodrug thereof as claimed in claim 1, and a pharmaceutically acceptable excipient.

20. The pharmaceutical composition as claimed in claim 19, wherein the pharmaceutical composition is an oral or topical pharmaceutical composition.

21. (canceled)

22. A method of treating or preventing a disease, disorder or condition in a subject, the method comprising the step of administering an effective amount of the compound or a pharmaceutically acceptable salt, solvate or prodrug thereof as claimed in claim 1 to the subject, thereby treating or preventing the disease, disorder or condition, optionally wherein the disease, disorder or condition is responsive to NLRP3 inhibition.

23. The method as claimed in claim 22, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia; and
(xvii) any disease where an individual has been determined to carry a germline or somatic non-silent mutation in NLRP3.

24. The method as claimed in claim 22, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

25. (canceled)

26. The method as claimed in claim 22, wherein the compound is administered as a pharmaceutical composition further comprising a pharmaceutically acceptable excipient.

27. A method of inhibiting NLRP3 in a subject, comprising administering the compound or a pharmaceutically acceptable salt, solvate or prodrug thereof as claimed in claim 1 to the subject thereby inhibiting NLRP3.

28. A method of analysing inhibition of NLRP3 or an effect of inhibition of NLRP3 by a compound, comprising contacting a cell or non-human animal with the compound or a pharmaceutically acceptable salt, solvate or prodrug thereof as claimed in claim 1, and analysing inhibition of NLRP3 or an effect of inhibition of NLRP3 in the cell or non-human animal by the compound.
